(19) United States
(12) Patent Application Publication
(10) Pub. No.: US 2007/0134249 A1
(43) Pub. Date: Jun. 14, 2007

(54) Combination therapy and antibody panels

(75) Inventors: Dan W. Denney, Redwood City, CA (US); Keri Marie Tate, Portola Valley, CA (US); Thomas P. Theriault, Palo Alto, CA (US)

Correspondence Address: Medlen & Carroll, LLP, Suite 350, 101 Howard Street, San Francisco, CA, 94105, US

(73) Assignees: Genitope Corporation, Redwood City, CA (US); Third Wave Technolgies

(21) Appl. No.: 11/297,168

(22) Filed: Dec. 8, 2005

(51) Int. Cl. A61K 39/395; C12Q 1/68; G01N 33/574; C12M 3/00

(52) U.S. Cl. 424/155.1; 435/7.23; 435/287.2; 435/6

(57) ABSTRACT

The present invention provides combination immunotherapy for Non-Hodgkin's Lymphoma. In one embodiment, the combination immunotherapy first provides for the administration of a monoclonal antibody directed to a non-idotypic portion of a lymphoma cell surface immunoglobulin (e.g. a framework region of a variable region). The combination immunotherapy next provides for the administration of an immunogenic composition comprising at least a portion of the same lymphoma cell surface immunoglobulin, whether an idiotypic portion or non-idiotypic portion.

### FIELD OF THE INVENTION

The present invention is related to combination immunotherapy for Non-Hodgkin's Lymphoma. In one embodiment, the combination immunotherapy first provides for the administration of a monoclonal antibody, or antibody fragment, directed to a non-idotypic portion of a lymphoma cell surface immunoglobulin (e.g. a framework region of a variable region). The combination immunotherapy next provides for the administration of an immunogenic composition comprising at least a portion of the same lymphoma cell surface immunoglobulin, whether an idiotypic portion or non-idiotypic portion.

### BACKGROUND OF THE INVENTION

Lymphomas

Lymphomas represent about 4% of the new cases of cancer diagnosed in the United States each year, making them the fifth most common cancer diagnosis and the fifth leading cause of cancer death. About 60,000 are diagnosed with lymphoma every year, of which about 90% are Non-Hodgkin Lymphomas (NHLs), with the remainder being Hodgkin Lymphoma (HL). In fact, while the incidence of most cancers is decreasing, lymphoma is one of only two tumors increasing in frequency, although the cause for this increase is unknown.

NHLs are a heterogeneous group of clonal neoplasms that arise from the lymphoid cell lineages. In the proposed WHO classification of NHL, the tumors are primarily classified according to: i) B- or T-cell lineage; ii) cyto-morphological appearance; iii) histopathological growth pattern; iv) immunophenotypic characteristics; and v) recurrent genetic aberrations. Some malignant lymphomas, predominantly those with slow growth characteristics and indolent course, may occasionally undergo spontaneous remissions. Veelken et al., “Vaccination Strategies In The Treatment Of Lyrnphomas” Oncology 62:187-200 (2002), herein incorporated by reference.

In general, the NHLs are divided into diseases that are indolent, aggressive, and very aggressive. The follicular lymphomas are the most common subtype of indolent NHL, representing about 30% of NHLs. While a variety of approaches were taken, no particular treatment clearly prolonged the survival of patients with advanced stage follicular NHL. Cheson, B. D., “What Is New In Lymphoma? CA: A Cancer Journal for Clinicians 54:260-272 (2004), herein incorporated by reference.

Lymphoma Treatments

Although, NHL responds initially to low dose chemotherapy and/or radiotherapy, relapses and treatment refraction occur after a period of months or years. Very high dose chemotherapy and/or radiotherapy with bone marrow or stem cell transplantation can induce longer remissions but unfortunately is substantially toxic, carries a high early mortality, and is not curative. Dermine et al., “Vaccine and antibody-directed T Cell Tumor Immunotherapy” Biochim Biophys ACTA 1704:11-35 (2004), herein incorporated by reference.

In B-cell lymphoma malignancies, a clonotypic surface immunoglobulin (Ig) expressed by malignant B-cells is known as an idiotype (Id) epitope. Id is a tumor-specific antigen and, therefore, provides a unique opportunity to target the tumor. See Miller et al., “Treatment of B cell lymphoma with monocloncal anti-idiotype antibody,” N. Engl. J Med. 306:517 (1982); Hamblin et al., “Preliminary experience in treating lymphocytic leukaemia with antibody to immunoglobulin idiotypes on the cell surfaces,” Br. J Cancer 42:495 (1980); Rankin et al, “Treatment of two patients with B cell lymphoma with monocloncal anti-idiotype antibodies,” Blood 65:1373 (1985); see generally Baskar et al., “Autologous Lymphoma Vaccines Induce Human T Cell Responses Against Multiple, Unique Epitopes: J Clin Invest. 113:1498-1510 (2004); all of which are herein incorporated by reference. There are a number of problems with the traditional anti-idiotype approach. Tumor cells are known to have the ability to endocytose surface idiotype plus attached antibody and thereby escape from antibody attack. Another problem that can occur is the continued somatic mutation of the variable region leading to a change in the idiotope. Alternatively, the tumor cell may simply down-regulate the idiotype epitopes. Gordon et al., “Mechanisms of tumor cell escape encountered in treating lymphocytic leukaemia with anti-idiotype antibody” Br J Cancer 49:547 (1984), herein incorporated by reference. Moreover, anti-idiotype antibodies are suggested to directly complex with secreted anti-idiotype proteins thereby reducing the therapeutic efficacy of monoclonal anti-idiotype antibodies. Meeker et al., “Antibodies to shared idiotypes as agents for analysis and therapy for human B cell tumors” Blood 68:430-436 (1986). In such cases, the secreted idiotype in the plasma binds the therapeutic antibody and prevents attachment to tumor cells. Stevenson et al., “Extracellular idiotypic immunoglobulin arising from human leukemic lymphocytes” J Exp Med 152:1484 (1980).

The monoclonal anti-idiotype approach has been largely abandoned in light of the development of specific monoclonal antibodies directed to CD-related receptor sites (i.e., RITUXIMAB). However, such antibodies eliminate healthy B-cells as well, compromising the ability of the patient to make a normal immune response. Moreover, recent reviews of numerous clinical research studies have concluded that all patients eventually become resistant to RITUXIMAB therapy. It was suggested that RITUXIMAB results in inadequate serum concentrations, loss of CD20 expression, or that tumor cells are inaccessible to the antibody. Cheson, B. D., “What Is New In Lymphoma? CA: A Cancer Journal for Clinicians 54:260-272 (2004).

What is needed, therefore, are immunotherapeutic compositions and in vivo methods which induce lymphoma tumor cell regression without inducing immunodeficiency or triggering tumor cell escape mechanisms, in patients that, for example, have not received prior anticancer therapy or are otherwise in need of such therapy.

### SUMMARY OF THE INVENTION

The present invention provides combination immunotherapy for Non-Hodgkin's Lymphoma and related diseases. In certain embodiments, the combination immunotherapy first provides for the administration of a monoclonal antibody, or antibody fragment, directed to a non-idotypic portion of a lymphoma cell surface immunoglobulin (e.g. a framework region of a variable region). The combination immunotherapy next provides for the administration of an immunogenic composition comprising at least a portion of the same lymphoma cell surface immunoglobulin, whether an idiotypic portion or non-idiotypic portion.

In certain embodiments, the present invention provides methods of treating a B-cell non-Hodgkin's Lymphoma in a human, the method comprising: a) administering to a subject (e.g. human) diagnosed with a B-cell non-Hodgkin's Lymphoma, a monoclonal antibody, or fragment thereof, reactive with an epitope of an immunoglobulin determined to be present on the human's non-Hodgkin's Lymphoma; and b) immunizing the human with at least a portion of the immunoglobulin present on the human's non-Hodgkin's Lymphoma.

In particular embodiments, the present invention provides methods of treating a B-cell non-Hodgkin's Lymphoma in a subject (e.g., human), the method comprising: a) administering to a subject diagnosed with a B-cell non-Hodgkin's Lymphoma, a humanized monoclonal antibody, or fragment thereof, reactive with a framework epitope of an immunoglobulin determined to be present on the human's non-Hodgkin's Lymphoma; and b) immunizing the subject with at least a portion of the immunoglobulin present on the human's non-Hodgkin's Lymphoma, the portion comprising an idiotypic epitope.

In some embodiments, the epitope of step (a) is a framework (FR) epitope. In other embodiments, the epitope of step (a) is within CDR1 or CDR2. In further embodiments, the epitope of step (a) includes part of the framework and part of a CDR (e.g. CDR1 or CDR2). In further embodiments, the portion of the immunoglobulin used in the immunizing of step (b) comprises an idiotypic epitope. In some embodiments, the idiotypic epitope is within CDR3. In particular embodiments, the monoclonal antibody or frament thereof of step (a) is not reactive with the idiotypic epitope.

In certain embodiments, the subject has measurable tumor burden prior to step (a) and exhibits at least a 25% reduction in tumor burden after step (a) (e.g. at least 25%, 30%, 40% or between 25-40%). In other embodiments, the subject has a measurable tumor burden prior to step (a) and exhibits at least a 50% reduction in tumor burden after step (a) (e.g. at least 50%, 60%, 70%, 80%, or 90%). In particular embodiments, the reduction in tumor burden is measured prior to the immunizing of step (b). In some embodiments, the administering of step (a) results in less than 25% depletion of normal B cells in the subject (e.g., less than 25%, less than 20%, less than 15%, less than 10% or less than 5%). In particular embodiments, the administering of step (a) results in less than 15% depletion of normal B cells in the subject.

In additional embodiments, the subject has not previously undergone an anti-non-Hodgkin's Lymphoma treatment regime. In other embodiments, the subject has not previously undergone anti-non-Hodgkin's Lymphoma chemotherapy. In further embodiments, the subject has not previously undergone anti-non-Hodgkin's Lymphoma radiation. In some embodiments, the subject has not previously undergone anti-non-Hodgkin's Lymphoma with a monoclonal antibody directed against a non-Ig molecule. In other embodiments, the human has not previously been treated with an anti-CD-20 antibody. In certain embodiments, the B-cell non-Hodgkin's Lymphoma is a member selected from the group consisting of low grade non-Hodgkin's Lymphoma, intermediate grade non-Hodgkin's Lymphoma, follicular lymphoma, Mantle cell lymphoma, and Burkitt's lymphoma.

In some embodiments, the monoclonal antibody or fragment thereof is a chimeric. In certain embodiments, the monoclonal antibody or fragment thereof is a humanized. In further embodiments, the monoclonal antibody or fragment thereof is a human antibody.

In particular embodiments, the present invention provides a panel of family specific antibodies comprising at least two, or at least three or at least four monoclonal antibodies, or fragments thereof, wherein each of the monoclonal antibodies reacts with at least two members of a variable region family.

In certain embodiments, one of the four monoclonal antibodies is reactive with a light chain variable region framework proteins in the VK3 family, wherein the monoclonal antibody does not cross-react with variable region proteins from non-VK3 families of variable regions. In other embodiments, the monoclonal antibody has immunoreactivity with VK3-20 and is unreactive with VK4-1.

In some embodiments, one of the four monoclonal antibodies is reactive with a heavy chain variable region framework proteins in the VH3 family, wherein the monoclonal antibody does not cross-react with variable region proteins from non-VH3 families of variable regions. In additional embodiments, the monoclonal antibody has immunoreactivity with VH3-48 and is unreactive with VK4-1.

In certain embodiments, one of the four monoclonal antibodies is reactive with a light chain variable region framework proteins in the VK4 family, wherein the monoclonal antibody does not cross-react with variable region proteins from non-VK4 families of variable regions. In particular embodiments, the monoclonal antibody has immunoreactivity with VK4-1 and is unreactive with VK3-20, VH3-48, and VH3-23.

In further embodiments, one of the four monoclonal antibodies is reactive with a light chain variable region framework proteins in the VL1 family, wherein the monoclonal antibody does not cross-react with variable region proteins from non-V1 families of variable regions. In some embodiments, the monoclonal antibody has immunoreactivity with VL1-51 and is unreactive with VK4-1.

In certain embodiments, the present invention provides methods of treating a patient having a B-cell non-Hodgkin's lymphoma, the lymphoma expressing an immunologic antigen receptor comprising a variable region, comprising: a) providing the panel of antibodies of described above or elsewhere herein; and b) treating the patient with a monoclonal antibody selected from the panel.

In some embodiments, the present invention provides a panel of antibodies or antibody fragments comprising: a) a first monoclonal antibody having immunoreactivity with VH3-48, the first monoclonal antibody being unreactive with VK3-20, VK4-1, and VH3-23; b) a second monoclonal antibody having immunoreactivity with VK3-20, the second monoclonal antibody being unreactive with VH3-48, VK4-1, and VH3-23; c) a third monoclonal antibody having immunoreactivity with VK4-1, the third monoclonal antibody being unreactive with VK3-20, VH3-48, and VH3-23; and d) a fourth monoclonal antibody having immunoreactivity with VH3-23, the fourth monoclonal antibody being unreactive with VK3-20, VK4-1, and VH3-48.

In certain embodiments, the present invention provides methods for classifying a B-cell non-Hodgkin's lymphoma of a patient, the lymphoma comprising cells expressing an immunologic antigen receptor comprising a variable region, comprising: a) contacting cells of the lymphoma with the panel of antibodies described above or elsewhere in the application; b) determining which of the antibodies of the panel bind to the cells; wherein the lymphoma is classified as belonging to a variable region family corresponding to the variable region recognized by antibodies that bind to the malignancy.

In some embodiments, the present invention provides methods of classifying a B-cell non-Hodgkin's lymphoma of a patient, the lymphoma comprising cells expressing an immunologic antigen receptor comprising a variable region, the method comprising: a) obtaining a polynucleotide sequence of the variable region of the lymphoma; b) comparing the polynucleotide sequence to panel of variable region reference sequences comprising a VH3-48 sequence, a VK3-20 sequence, a VK4-1 sequence, and a VH3-23 sequence (or other highly prevalent sequences according to Table 1); c) identifying the reference sequence having the highest sequence similarity to the variable region of the lymphoma; wherein the lymphoma is classified as belonging to a variable region family corresponding to the reference sequence having the highest sequence similarity.

In further embodiments, the present invention provides methods for treating a patient having a B-cell non-Hodgkin's lymphoma of a patient, the lymphoma expressing an immunologic antigen receptor comprising a variable region, comprising: a) providing the panel of antibodies of described above; and b) treating the patient with a monoclonal antibody selected from the panel.

In some embodiments, the present invention provides a panel of antibodies or antibody fragments comprising: a) a first monoclonal antibody having immunoreactivity with VH3-48, the first monoclonal antibody being unreactive with VK3-20, VK4-1, and VH3-23; b) a second monoclonal antibody having immunoreactivity with VK3-20, the second monoclonal antibody being unreactive with VH3-48, VK4-1, and VH3-23; c) a third monoclonal antibody having immunoreactivity with VK4-1, the third monoclonal antibody being unreactive with VK3-20, VH3-48, and VH3-23; d) a fourth monoclonal antibody having immunoreactivity with VH3-23, the fourth monoclonal antibody being unreactive with VK3-20, VK4-1, and VH3-48; e) a fifth monoclonal antibody having immunoreactivity with VL1-51, the fifth monoclonal antibody being unreactive with VK4-1.

In particular embodiments of the panels, immunoreactivity is specific to one chain of the BCR. In other embodiments, of the panels, immunoreactivity is specific for the framework regions of the defined variable region genes and extends into one or more of the CDR1 and/or CDR2 of the same chain. In some embodiments of the panels, the immunoreactivity includes only the framework regions. In further embodiments of the panels, the immunoreactive epitopes are largely unchanged from the corresponding germline sequence.

In other embodiments, the present invention provides methods for treating a patient having a B-cell non-Hodgkin's lymphoma of a patient, the lymphoma expressing an immunologic antigen receptor comprising a variable region, comprising: a) providing the panel of antibodies (e.g. as described above); and b) treating the patient with a monoclonal antibody selected from the panel.

In some embodiments, the present invention provides a composition comprising at least one of the following: a) a first monoclonal antibody having immunoreactivity with VH3-48, the first monoclonal antibody being unreactive with VK3-20, VK4-1, and VH3-23; b) a second monoclonal antibody having immunoreactivity with VK3-20, the second monoclonal antibody being unreactive with VH3-48, VK4-1, and VH3-23; c) a third monoclonal antibody having immunoreactivity with VK4-1, the third monoclonal antibody being unreactive with VK3-20, VH3-48, and VH3-23; and d) a fourth monoclonal antibody having immunoreactivity with VH3-23, the fourth monoclonal antibody being unreactive with VK3-20, VK4-1, and VH3-48.

In certain embodiments, the present invention provides methods for classifying a B-cell Non-Hodgkin's lymphoma of a patient, the lymphoma comprising cells expressing an immunologic antigen receptor comprising a variable region, comprising: a) contacting cells of the lymphoma with a panel of antibodies (e.g. described above); b) determining which of the antibodies of the panel bind to the cells; wherein the lymphoma is classified as belonging to a variable region family corresponding to the variable region recognized by antibodies that bind to the malignancy.

In particular embodiments, the present invention provides methods of classifying a B-cell Non-Hodgkin's lymphoma of a patient, the lymphoma comprising cells expressing an immunologic antigen receptor comprising a variable region, the method comprising: a) obtaining a polynucleotide sequence of the variable region of the lymphoma; b) comparing the polynucleotide sequence to panel of variable region reference sequences comprising a VH3-48 sequence, a VK3-20 sequence, a VK4-1 sequence, and a VH3-23 sequence; and c) identifying the reference sequence having the highest sequence similarity to the variable region of the lymphoma; wherein the lymphoma is classified as belonging to a variable region family corresponding to the reference sequence having the highest sequence similarity.

In further embodiments, the present invention provides methods to measure immunization potency by using variable region-specific mAbs to compare purified framework epitope protein to KLH-conjugated framework epitope protein. In certain embodiments, a strong decrease or loss of immunoreactivity indicates over-conjugation.

In some embodiments, the present invention provides methods of classifying comprising; a) obtaining a sample from a patient comprising a tumor associated idiotypic protein, wherein the idiotypic protein comprises a heavy chain variable region and a light or kappa chain variable region; and b) classifying the heavy or light/kappa chain variable region of the idiotypic protein as belonging to a particular variable region family or family member (e.g., using sequencing or an antibody panel). In further embodiments, the method further comprises: c) treating the patient with a composition comprising a monoclonal antibody reactive with the particular heavy or light/kappa chain variable region family or family member that is determined in step b).

In particular embodiments, the present invention provides methods for patient classification of immunologic malignancies characterized by malignant cells expressing an immunologic antigen receptor, the method comprising: obtaining a malignancy polynucleotide sequence of the variable region of the immunologic receptor from a sample comprising the malignant cells; comparing the polynucleotide sequence to reference sequences of the immunologic antigen receptor; identifying the reference sequence having the highest sequence similarity to the malignancy polynucleotide sequence; wherein the patient is classified as belonging to a variable region family corresponding to the reference sequence having the highest sequence similarity to the malignancy polynucleotide sequence. In some embodiments, the reference sequence(s) are human germline sequences. In further embodiments, the malignant polynucleotide sequence is obtained by anchored PCR type methods or other methods described in the Examples below (see, e.g. Example 1). In certain embodiments, the sample is a biopsy sample. In additional embodiments, the sample comprises less than about 50% malignant cells. In further embodiments, the sample comprises less than about 10% malignant cells. In other embodiments, the reference sequences comprise at least about 10 or 15 of the germline variable region sequences of the immunologic receptor.

In some embodiments, the malignancy polynucleotide sequence is classified as belonging to a variable region family when the polynucleotide sequence differs by less than about 15% or 10% of the nucleotides from the reference sequence. In further embodiments, the immunologic receptor is an immunoglobulin. In other embodiments, the malignancy is a member selected from the group consisting B-cell non-Hodgkin's lymphoma (NHL) and B-cell leukemia. In particular embodiments, the B-cell NHL is selected from the group consisting of follicular lymphoma, diffuse large B-cell lymphoma, small lymphocytic lymphoma, mantle cell lymphoma, marginal zone B-cell lymphoma, MALT type, primary mediastinal large B-cell lymphoma, B-cell lymphoblastic lymphoma, Burkitt-like lymphoma, marginal zone B-cell lymphoma, nodal type, lymphoplasmacytic lymphoma, Burkitt's lymphoma. In further embodiments, the immunologic receptor is a T cell antigen receptor.

In certain embodiments, the method further comprises administering to the patient an antibody that reacts with at least two (or at least three or four) members of the variable region family. In other embodiments, the method further comprises vaccinating the patient with at least a portion of the immunologic antigen receptor.

In some embodiments, the present invention provides methods for patient classification of immunologic malignancies characterized by malignant cells expressing an immunologic antigen receptor, the method comprising: contacting a sample comprising the malignant cells with a panel of family specific antibodies, wherein each of the antibodies reacts with at least two (or at least three or four) members of a variable region family; determining which of the antibodies bind to the malignant cells; wherein the patient is classified as belonging to a variable region family corresponding to the variable region recognized by antibodies that bind to the malignancy.

In further embodiments, the present invention provides compositions comprising a monoclonal antibody, or antibody fragment, reactive with a light chain variable region framework proteins in the LV1 family, wherein the monoclonal antibody does not cross-react with variable region proteins from non-LV1 families of variable regions.

In some embodiments, the present invention provides compositions comprising a monoclonal antibody, or antibody fragment, reactive with a light chain variable region framework proteins in the LV2 family, wherein the monoclonal antibody does not cross-react with variable region proteins from non-LV2 families of variable regions.

In other embodiments, the present invention provides compositions comprising a monoclonal antibody, or antibody fragment, reactive with a light chain variable region framework proteins in the HV3 family, wherein the monoclonal antibody does not cross-react with variable region proteins from non-HV3 families of variable regions.

In certain embodiments, the present invention provides compositions comprising a monoclonal antibody, or antibody fragment, reactive with a light chain variable region framework proteins in the HV4 family, wherein the monoclonal antibody does not cross-react with variable region proteins from non-HV4 families of variable regions.

In some embodiments, the present invention provides compositions comprising a monoclonal antibody, or antibody fragment, reactive with a light chain variable region framework proteins in the KV3 family, wherein the monoclonal antibody does not cross-react with variable region proteins from non-KV3 families of variable regions.

In particular embodiments, the present invention provides compositions comprising a monoclonal antibody, or antibody fragment, reactive with a light chain variable region framework proteins in the KV4 family, wherein the monoclonal antibody does not cross-react with variable region proteins from non-KV4 families of variable regions.

In some embodiments, the present invention provides compositions comprising a monoclonal antibody, or fragment thereof, reactive with heavy chain variable region framework proteins classified as family member HV3-23, wherein the monoclonal antibody does not cross-react with variable region proteins from non-HV3-23 family member variable regions.

In some embodiments, the present invention provides compositions comprising a monoclonal antibody, or fragment thereof, reactive with heavy chain variable region framework proteins classified as family member KV4-1, wherein the monoclonal antibody does not cross-react with variable region proteins from non-KV4-1 family member variable regions.

### DESCRIPTION OF THE FIGURES

FIG. 1 shows the amino acid sequence of the heavy and light (lambda or kappa) chain variable regions from five PIN idiotypic proteins that were used as immunogens in Example 2 below. In particular, FIG. 1A shows the amino acid sequences of the heavy (SEQ ID NO:1) and light chain (SEQ ID NO:2) variable regions from PIN574; FIG. 1B shows the amino acid sequences of the heavy (SEQ ID NO:3) and kappa chain (SEQ ID NO:4) variable regions from PIN149; FIG. 1C shows the amino acid sequences of the heavy (SEQ ID NO:5) and light chain (SEQ ID NO:6) variable regions from PIN116; FIG. 1D shows the amino acid sequences of the heavy (SEQ ID NO:7) and light chain (SEQ ID NO:8) variable regions from PIN647; and FIG. 1E shows the amino acid sequences of the heavy (SEQ ID NO:9) and kappa chain (SEQ ID NO:10) variable regions from PIN628.

FIG. 2 shows the results of an ELISA testing for HV3-23 specific mAbs from fusions 13 and 14 as described in Example 2.

FIG. 3 shows the results of an ELISA testing for KV4-1 specific mAbs from fusions 13 and 14 as described in Example 2.

FIG. 4 shows the results of an ELISA testing for LV1 and LV2 specific mAbs from fusions 15, 16, 17 and 18 as described in Example 2.

FIG. 5 shows the results of an ELISA testing for KV4-1 specific mAbs from fusions 20 and 21 as described in Example 2.

FIG. 6 shows the amino acid sequence of mAb clone 3C9. FIG. 6A shows the amino acid sequence (SEQ ID NO:11) and the nucleic acid sequence (SEQ ID NO:12) of the heavy chain variable region from mAb clone 3C9. FIG. 6B shows the amino acid sequence (SEQ ID NO:13) and the nucleic acid sequence (SEQ ID NO:14) of the light chain variable region from mAb clone 3C9. The three CDRs in each of these sequences are underlined.

FIG. 7 shows the amino acid sequence of mAb clone 10H7. FIG. 7A shows the amino acid sequence (SEQ ID NO:15) and the nucleic acid sequence (SEQ ID NO:16) of the heavy chain variable region from mAb clone 10H7. FIG. 7B shows the amino acid sequence (SEQ ID NO:17) and the nucleic acid sequence (SEQ ID NO:18) of the light chain variable region from mAb clone 10H7. The three CDRs in each of these sequences are underlined.

FIG. 8 shows the amino acid sequence of mAb clone 12C3. FIG. 8A shows the amino acid sequence (SEQ ID NO:19) and the nucleic acid sequence (SEQ ID NO:20) of the heavy chain variable region from mAb clone 12C3. FIG. 8B shows the amino acid sequence (SEQ ID NO:21) and the nucleic acid sequence (SEQ ID NO:22) of the light chain variable region from mAb clone 12C3. The three CDRs in each of these sequences are underlined.

FIG. 9 shows the amino acid sequence of mAb clone 20H5. FIG. 9A shows the amino acid sequence (SEQ ID NO:23) and the nucleic acid sequence (SEQ ID NO:24) of the heavy chain variable region from mAb clone 20H5. FIG. 9B shows the amino acid sequence (SEQ ID NO:25) and the nucleic acid sequence (SEQ ID NO:126) of the light chain variable region from mAb clone 20H5. The three CDRs in each of these sequences are underlined.

FIG. 10 shows the amino acid sequence of mAb clone 15E8. FIG. 10A shows the amino acid sequence (SEQ ID NO:27) and the nucleic acid sequence (SEQ ID NO:28) of the heavy chain variable region from mAb clone 15E8. FIG. 10B shows the amino acid sequence (SEQ ID NO:29) and the nucleic acid sequence (SEQ ID NO:30) of the light chain variable region from mAb clone 15E8. The three CDRs in each of these sequences are underlined.

FIG. 11 shows the amino acid sequence of mAb clone 4H11. FIG. 11A shows the amino acid sequence (SEQ ID NO:31) and the nucleic acid sequence (SEQ ID NO:32) of the heavy chain variable region from mAb clone 4H11. FIG. 11B shows the amino acid sequence (SEQ ID NO:33) and the nucleic acid sequence (SEQ ID NO:34) of the light chain variable region from mAb clone 4H11. The three CDRs in each of these sequences are underlined.

FIG. 12 shows the results of an ELISA testing for HV4- and KV3-11-specific mAbs from fusion 22 as described in Example 2.

FIG. 13 shows the results of an ELISA testing for KV1-5- and KV1-specific mAbs from fusion 23 as described in Example 2.

FIG. 14 shows the amino acid sequence for eight V regions, four heavy chain and four light chains, used to generate four human-mouse chimera idiotype proteins used as immunogens in Example 2 below: 14A) PIN1155 HV4-34 (SEQ ID NO:67) and PIN609 KV3-11 (SEQ ID NO:68); 14B) PIN655 HV3-7 (SEQ ID NO:69) and PIN1092 KV1-5 (SEQ ID NO:70); 14C) PIN662 HV3-48 (SEQ ID NO:71) and PIN737 KV3-20 (SEQ ID NO:72); 14D) PIN913 HV4-59 (SEQ ID NO:73) and PIN1062 KV1-39 (SEQ ID NO:74).

FIG. 15 shows the antisera screening by ELISA for the three mice used in fusions 13 (Study Number 23) in white and four mice used in fusion 14 (Study Number 24) in black. All mice were immunized with PIN149/149 chimera Id protein and all antisera are tested at the same dilution for this experiment. On the X axis data are grouped by the individual Id proteins with columns for each mouse in each study. The absorbency units on the Y axis represent relative intensity for different animals' sera reactivity against the different fully human Id proteins. Antisera were tested against the immunogen and HV3-23 (N=13), KV4-1 (N=8), and HV3-23/KV4-1 (N=3) Id proteins (same V region family members are grouped by brackets). Data from antisera screening against alternative family member derived Id proteins are not shown. The numbers under the X axis represent the number of hybridomas that recognize a particular Id protein . There were a total of seven HV3-23- and eight KV4-1-specific hybridomas generated from these two fusions (see Antisera screening in Example 2 for more details).

FIG. 16 shows the antisera screening by ELISA for the three BALB/c mice in white and three C3H-HeN mice in black for Study Number 33. All mice were immunized with PIN1607/149 chimera Id protein and all antisera are tested at the same dilution for this experiment. On the X axis data are grouped by the individual Id proteins with columns for each mouse in the study. The absorbency units on the Y axis represent relative intensity for different animals' sera reactivity against the different fully human Id proteins. Antisera were tested against the immunogen and HV3-48 (N=25), KV4-1 (N=10), and HV3-48/KV4-1 (N=1) Id proteins (same V region family members are grouped by brackets). Data from antisera screening against alternative family member derived Id proteins are not shown (see Antisera screening in Example 2 for more details).

FIG. 17 shows the antisera screening by ELISA for the three BALB/c mice in white and two C3H-HeN mice in black for fusion 22. All mice were immunized with PIN1155/609 chimera Id protein and all antisera are tested at the same dilution for this experiment. On the X axis data are grouped by the individual Id proteins with columns for each mouse in the study. The absorbency units on the Y axis represent relative intensity for different animals' sera reactivity against different fully human Id proteins. Antisera were tested against HV4-34 (N=15), HV4 (N=4), KV3-11 (N=11), and HV3 (N=2), and HV4-34/KV3-11 (N=1) Id proteins (same V region families are grouped by brackets). Data from antisera screening against alternative family member derived Id proteins are not shown (see Antisera screening in Example 2 for more details).

FIG. 18 shows the antisera screening by ELISA for the three BALB/c mice in white and three C3H-HeN mice in black for fusion 23. All mice were immunized with PIN655/1092 chimera Id protein and all antisera are tested at the same dilution for this experiment. On the X axis data are grouped by the individual Id proteins with columns for each mouse in the study. The absorbency units on the Y axis represent relative intensity for different animals' sera reactivity against different fully human Id proteins. Antisera screening is shown for KV1-5 (N=21) and KV1 (N=8) Id proteins only (V regions are grouped by brackets for KV1-5 and all KV1 Id proteins). HV3-7 and HV3 antisera screening results are not shown. There were a total for 15 KV1-specific hybridomas generated from this fusion. The large bold numbers under the germline V regions are the number of hybridomas that recognize each Id protein (see Antisera screening in Example 2 for more details).

FIG. 19 shows the antisera screening by ELISA for the three BALB/c mice in white and three C3H-HeN mice in black for Fusion 25. All mice were immunized with PIN913/1062 chimera Id protein and all antisera are tested at the same dilution for this experiment. On the X axis data are grouped by the individual Id proteins with columns for each mouse in the study. The absorbency units on the Y axis represent relative intensity for different animals' sera reactivity against different fully human Id proteins. Antisera were tested against HV4-59 (N=18), HV4 (N=7), KV1-39 (N=15), HV1 (N=2), HV4/KV1 (N=5) Id proteins (same V region families are grouped by brackets). Data from antisera screening against alternative family member derived Id proteins are not shown (see Antisera screening in Example 2 for more details).

### DEFINITIONS

To facilitate an understanding of the invention, a number of terms are defined below.

As used herein “idiotype” refers to an epitope in the hypervariable region of an immunoglobulin chain, including but not limited to an epitope formed by contributions from both the light chain and heavy chain CDRs. A “non-idiotypic portion” refers to an epitope located outside the hypervariable regions, such as the framework regions.

As used herein “immunoglobulin” refers to any of a group of large glycoproteins that are secreted by plasma cells and that function as antibodies in the immune response by binding with specific antigens. The specific antigen bound by an immunoglobulin may or may not be known. There are five classes of immunoglobulins: IgA, IgD, IgE, IgG, and IgM.

The term “antibody,” as used herein, is intended to refer to immunoglobulin molecules comprised of four polypeptide chains, two heavy (H) chains and two light (L) chains (lambda or kappa) inter-connected by disulfide bonds. An antibody has a known specific antigen with which it binds. Each heavy chain of an antibody is comprised of a heavy chain variable region (abbreviated herein as HCVR, HV or VH) and a heavy chain constant region. The heavy chain constant region is comprised of three domains, CH1, CH2 and CH3. Each light chain is comprised of a light chain variable region (abbreviated herein as LCVR or VL or KV or LV to designate kappa or lambda light chains) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each variable region (VH or VL) contains 3 CDRs, designated CDR1, CDR2 and CDR3. Each variable region also contains 4 framework sub-regions, designated FR1, FR2, FR3 and FR4.

As used herein, the term “antibody fragments” refers to a portion of an intact antibody. Examples of antibody fragments include, but are not limited to, linear antibodies, single-chain antibody molecules, Fv, Fab and F(ab′)₂ fragments, and multispecific antibodies formed from antibody fragments. The antibody fragments preferably retain at least part of the heavy and/or light chain variable region.

As used herein, the terms “complementarity determining region” and “CDR” refer to the regions that are primarily responsible for antigen-binding. There are three CDRs in a light chain variable region (CDRL1, CDRL2, and CDRL3), and three CDRs in a heavy chain variable region (CDRH1, CDRH2, and CDRH3). The particular designation in the art for the exact location of the CDRs varies depending on what definition is employed. Preferably, the IMGT designations are used, which uses the following designations for both light and heavy chains: residues 27-38 (CDR1), residues 56-65 (CDR2), and residues 105-116 (CDR3); see Lefrance, MP, The Immunologist, 7:132-136, 1999, herein incorporated by reference. The residues that make up the six CDRs have also been characterized by Kabat and Chothia as follows: residues 24-34 (CDRL1), 50-56 (CDRL2) and 89-97 (CDRL3) in the light chain variable region and 31-35 (CDRH1), 50-65 (CDRH2) and 95-102 (CDRH3) in the heavy chain variable region; Kabat et al., (1991) Sequences of Proteins of Immunological Interest, 5th Ed. Public Health Service, National Institutes of Health, Bethesda, Md., herein incorporated by reference; and residues 26-32 (CDRL1), 50-52 (CDRL2) and 91-96 (CDRL3) in the light chain variable region and 26-32 (CDRH1), 53-55 (CDRH2) and 96-101 (CDRH3) in the heavy chain variable region; Chothia and Lesk (1987) J. Mol. Biol. 196: 901-917, herein incorporated by reference. Unless otherwise specified, the terms “complementarity determining region” and “CDR” as used herein, include the residues that encompass IMGT, Kabat and Chothia definitions. Also, unless specified, as used herein, the numbering of CDR residues is according to IMGT.

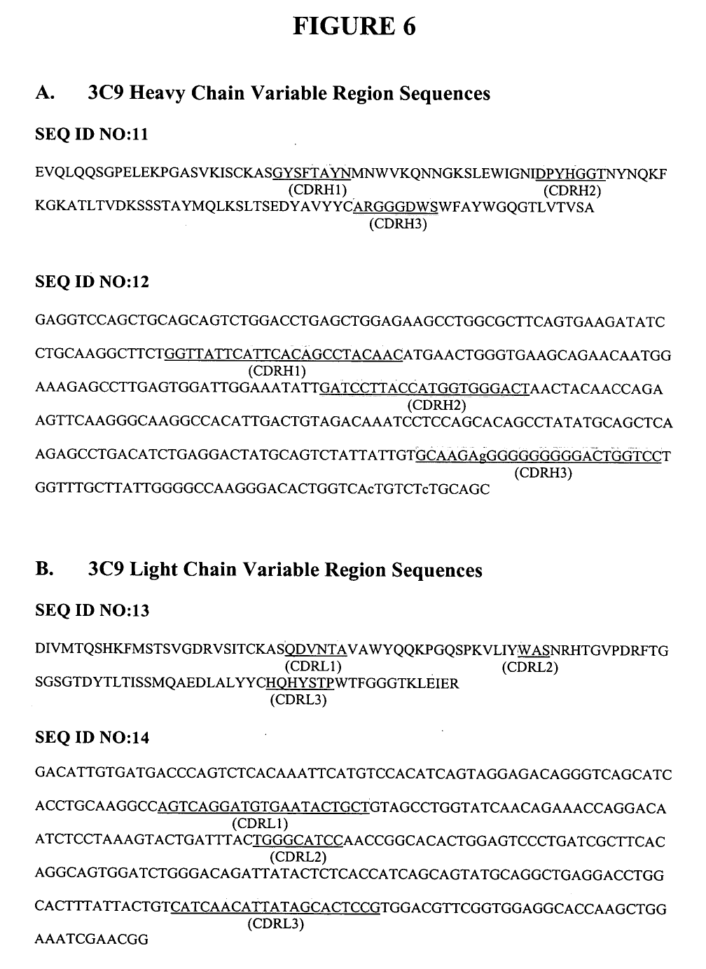

As used herein, the term “framework” refers to the residues of the variable region other than the CDR residues as defined herein. There are four separate framework sub-regions that make up the framework: FR1, FR2, FR3, and FR4 (See non-underlined regions in FIGS. 6-11). In order to indicate if the framework sub-region is in the light or heavy chain variable region, an “L” or “H” may be added to the sub-region abbreviation (e.g., “FRL1” indicates framework sub-region 1 of the light chain variable region). Unless specified, the numbering of framework residues is according to IMGT.

As used herein, “antigen” refers to any substance that, when introduced into a body, e.g., of a patient or subject, stimulates an immune response such as the production of an antibody that recognizes the antigen.

As used herein, the term “immunogenic composition” refers to a composition comprising an antigen.

As used herein, the term “vaccine” refers to a composition comprising an antigen for use as a therapy or treatment to induce an immune response. Vaccines may be used both prophylactically (for prevention of disease) and therapeutically (for the treatment of existing disease). For example, with respect to cancer therapies, a therapeutic vaccine would generally be given to a cancer patient to induce an immune response to fight the cancer, e.g., by attacking the patient's malignant cells, while a prophylactic vaccine would generally be given to an individual who does not have a particular type of cancer to induce an immune response to prevent that type of cancer, e.g., by attacking viruses known to cause that type of cancer.

The term “passive immunotherapy” as used herein refers to therapeutic treatment of a subject or patient using immunological agents such as antibodies (e.g., monoclonal antibodies) produced outside a subject or patient, without the purpose of inducing the subject or patient's immune system to produce a specific immune response to the therapeutic agent.

The term “active immunotherapy” as used herein refers to therapeutic treatment of a subject or patient to induce the subject or patient's immune system to produce a specific immune response, e.g., to a protein derived from a malignant cell. In preferred embodiments, the immunogenic composition used in active immunotherapy comprises one or more antigens derived from a subject's malignant cells. In some particularly preferred embodiments, the immunogenic agent comprises at least a portion of an immunoglobulin derived from a subject's malignant cell. It is understood by those of skill in the art that, as used in active immunotherapy, an immunoglobulin derived from a patient or subject's malignant cell is generally used as an antigen, not as an antibody intended to act as a therapeutic agent in passive immunotherapy.

As used herein, the terms “subject” and “patient” refer to any animal, such as a mammal like a dog, cat, bird, livestock, and preferably a human.

As used herein, the terms “an oligonucleotide having a nucleotide sequence encoding a polypeptide,” “polynucleotide having a nucleotide sequence encoding a polypeptide,” and “nucleic acid sequence encoding a peptide” means a nucleic acid sequence comprising the coding region of a particular polypeptide. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc., or a combination of both endogenous and exogenous control elements.

As used herein, the terms “complementary” or “complementarity” are used in reference to polynucleotides (i.e., a sequence of nucleotides) related by the base-pairing rules. For example, the sequence “5′-A-G-T 3”, is complementary to the sequence “3-T-C-A-5′”. Complementarity may be “partial”, in which only some of the nucleic acids' bases are matched according to the base pairing rules, or, there may be “complete” or “total” complementarity between the nucleic acids. The degree of complementarity between nucleic acid strands has significant effects on the efficiency and strength of hybridization.

As used herein, the term “the complement of” a given sequence is used in reference to the sequence that is completely complementary to the sequence over its entire length. For example, the sequence 5′-A-G-T-A-3′ is “the complement” of the sequence 3′-T-C-A-T-5′. The present invention also provides the complement of the sequences described herein (e.g., the complement of the nucleic acid sequences in SEQ ID NOs: 11-34 or the complement of the CDRs in these sequences).

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent [50× Denhardt's contains per 500 ml: 5 g Ficoll (Type 400, Pharmacia), 5 g BSA (Fraction V; Sigma)] and 100 μg/ml denatured salmon sperm DNA, followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE, 0.5% SDS, 5× Denhardt's reagent and 100 μg/ml denatured salmon sperm DNA, followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE, 0.1% SDS, 5× Denhardt's reagent and 100 g/ml denatured salmon sperm DNA, followed by washing in a solution comprising 5×SSPE, 0.1% SDS at 42° C. when a probe of about 500 nucleotides in length is employed.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated (e.g. host cell proteins).

As used herein, the terms “portion” when used in reference to a nucleotide sequence (as in “a portion of a given nucleotide sequence”) refers to fragments of that sequence. The fragments may range in size from ten nucleotides to the entire nucleotide sequence minus one nucleotide (e.g., 10 nucleotides, 20, 30, 40, 50, 100, 200, etc.).

As used herein, the term “portion” when in reference to an amino acid sequence (as in “a portion of a given amino acid sequence”) refers to fragments of that sequence. The fragments may range in size from six amino acids to the entire amino acid sequence minus one amino acid (e.g., 6 amino acids, 10, 20, 30, 40, 75, 200, etc.).

As used herein, the term “purified” or “to purify” refers to the removal of contaminants from a sample. For example, monoclonal antibodies reactive with a framework epitope of an immunoglobulin may be purified by removal of contaminating non-immunoglobulin proteins; they are also purified by the removal of immunoglobulins that do not bind to the same antigen. The removal of non-immunoglobulin proteins and/or the removal of immunoglobulins that do not bind the particular antigen results in an increase in the percentage of antigen specific immunoglobulins in the sample. In another example, recombinant antigen-specific polypeptides are expressed in bacterial host cells and the polypeptides are purified by the removal of host cell proteins; the percentage of recombinant antigen-specific polypeptides is thereby increased in the sample.

As used herein, the term “treatment” refers to both therapeutic treatment and prophylactic or preventative measures. Those in need of treatment include those already with the disorder as well as those in which the disorder is to be prevented.

The phrase “under conditions such that the symptoms are reduced” refers to any degree of qualitative or quantitative reduction in detectable symptoms of any disease treatable by monoclonal antibodies reactive with a non-idiotypic variable region (e.g., framework) epitope of an immunoglobulin, including but not limited to, a detectable impact on the rate of recovery from disease (e.g., rate of weight gain), or the reduction of at least one of the symptoms normally associated with the particular disease.

The terms “affinity”, “binding affinity” and “K_{d}” refer to the equilibrium dissociation constant (expressed in units of concentration) associated with each monoclonal antibody reactive with a non-idiotypic variable region (e.g., framework) epitope of an immunoglobulin-ligand complex. The binding affinity is directly related to the ratio of the off-rate constant (generally reported in units of inverse time, e.g., seconds⁻¹) to the on-rate constant (generally reported in units of concentration per unit time, e.g., molar/second). The binding affinity may be determined by, for example, an ELISA assay, kinetic exclusion assay or surface plasmon resonance. It is noted that certain epitopes can occur repetitively (multivalent) on a cell surface and that the dissociation constant (koff) for the binding of an antibody to a repetitive epitope may be greatly diminished over the dissociation constant for the reaction of the same antibody with the corresponding ligand in univalent form. The diminished dissociation constant arises because when one antibody-ligand bond dissociates, other bonds hold the bivalent (or multivalent) antibody to the multivalent ligand, allowing the dissociated bond to form again. The dissociation constant for the reaction between bivalent (or multivalent) antibody and multivalent ligand has been termed the functional affinity to contrast it with intrinsic affinity, which is the association constant for an antibodies representative individual site.

The terms “dissociation”, “dissociation rate” and “k_{off}” as used herein, are intended to refer to the off rate constant for dissociation of a monoclonal antibody reactive with a non-idiotypic variable region (e.g., framework) epitope of an immunoglobulin from the antibody/antigen complex.

The terms “association”, “association rate” and “kₒₙ” as used herein, are intended to refer to the on rate constant for association of a monoclonal antibody reactive with a non-idiotypic variable region epitope (e.g. framework epitope) of an immunoglobulin with an antigen to form an antibody/antigen complex.

As used herein, “humanized” forms of non-human (e.g., murine) antibodies are antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are generally made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody may also comprise at least a portion of an immunoglobulin constant region (Fc), typically that of a human immunoglobulin. Examples of methods used to generate humanized antibodies are described in U.S. Pat. No. 5,225,539 to Winter et al. (herein incorporated by reference).

Importantly, early methods for humanizing antibodies often resulted in antibodies with lower affinity than the non-human antibody starting material. More recent approaches to humanizing antibodies address this problem by making changes to the CDRs. See U.S. Patent Application Publication No. 20040162413, hereby incorporated by reference. In some embodiments, the present invention provides an optimized heteromeric variable region (e.g. that may or may not be part of a full antibody other molecule) having equal or higher antigen binding affinity than a donor heteromeric variable region, wherein the donor heteromeric variable region comprises three light chain donor CDRs, and wherein the optimized heteromeric variable region comprises: a) a light chain altered variable region comprising; i) four unvaried human germline light chain framework regions, and ii) three light chain altered variable region CDRs, wherein at least one of the three light chain altered variable region CDRs is a light chain donor CDR variant, and wherein the light chain donor CDR variant comprises a different amino acid at only one, two, three or four positions compared to one of the three light chain donor CDRs (e.g. the at least one light chain donor CDR variant is identical to one of the light chain donor CDRs except for one, two, three or four amino acid differences).

### DESCRIPTION OF THE INVENTION

The present invention provides combination immunotherapy for Non-Hodgkin's Lymphoma. In certain embodiments, the combination immunotherapy first provides for the administration of a monoclonal antibody (e.g. a fully human, a chimeric or otherwise humanized antibody) directed to a non-idotypic portion (e.g. a framework region) of a lymphoma cell surface immunoglobulin (e.g. a framework region of a variable region). The combination immunotherapy next provides for the administration of an immunogenic composition (vaccine) comprising at least a portion of the same lymphoma cell surface immunoglobulin, whether an idiotypic portion or non-idiotypic portion. In certain embodiments, an idiotypic portion is used for the an immunogenic composition and the antibody of the first step is not reactive with the material used in the immunogenic composition. In other embodiments, an idiotypic portion is used for the vaccine and the antibody or antibody fragment of the first step is reactive with the material used in the immunogenic composition.

I. Variable Region Family Member or Family Specific Monoclonal Antibodies

In certain embodiments, the present invention provides methods for generating and using human immunoglobulin heavy and light chain variable region (IGHV, IGLCV, and IGKV; herein referred to as HV, LV, and KV respectively) family- and family member-specific mouse monoclonal antibodies (mAbs) or fragements thereof. In certain embodiments, the mAb or mAb fragments recognize patient non-idiotypic variable region epitopes (e.g. framework epitopes) that may be used in diagnostic and therapeutic applications for treating patients with B-cell lymphoma or related diseases. Through molecular biological approaches, such monoclonal antibodies or fragments thereof can be humanized.

In other embodiments, the mAb may be used as an analytical reagent including, but not limited to, i) discriminating among subsets of patient non-idiotyping variable region epitopes (e.g. framework epitopes) during manufacturing runs (e.g., for example, in processing controls); ii) for measuring potency of variable region epitopes preceding and following protein modification (for example, KLH conjugation), and iii) determining the quantity of specific variable regions in complex mixtures.

In certain embodiments, the present invention provides method for obtaining a panel of mAbs having, for example, specificity differences that encompass a broad range of variable region types. In certain embodiments, each antibody is reactive with a different framework epitope.

The present invention provides a method for: i) identifying the frequency of patient variable region useage (e.g., as shown in Table 1) within a representative portion of a cancer population; ii) creating a panel of antibodies directed to react with at least 40% percent of the variable regions used (and more preferably, at least 60%, even more preferably at least 75%, and most preferably greater than 90% and up to 100%).

It is contemplated that many different recombinant forms of the immungen and many different screening approaches may be used to obtain mAbs. If desired, antibodies can be raised to all of the different variable regions observed in a representative group (e.g. a group of at least 300 cancer patients) of a cancer population. On the other hand, it is not necessary that reactivity with every variable region be achieved. In one embodiment, the present invention contemplates a panel of as few as three or four or five antibodies that collectively react with at least 25 to 35% or at least 40% of the variable regions used (i.e. observed in a representative group of cancer patients). For example, in one embodiment, the present invention contemplates a panel of antibodies comprising: a first monoclonal antibody or fragment thereof having immunoreactivity with sIg derived from the VH3-48 family member; a second monoclonal antibody or fragment thereof having immunoreactivity with sIg derived from the VK3-20 family member; a third monoclonal antibody or fragment thereof having immunoreactivity with sIg derived from the VK4-1 family member; and/or a fourth monoclonal antibody or fragment thereof having immunoreactivity with sIg derived from the VH3-23 family member. In another embodiment, the present invention contemplates a panel comprising a first monoclonal antibody or fragment thereof having immunoreactivity with VH3-48, said first monoclonal antibody being unreactive with VK3-20, VK4-1, and VH3-23; a second monoclonal antibody or fragment thereof having immunoreactivity with VK3-20, said second monoclonal antibody being unreactive with VH3-48, VK4-1, and VH3-23; a third monoclonal antibody or fragment thereof having immunoreactivity with VK4-1, said third monoclonal antibody being unreactive with VK3-20, VH3-48, and VH3-23; and/ora fourth monoclonal antibody or fragment thereof having immunoreactivity with VH3-23, said fourth monoclonal antibody being unreactive with VK3-20, VK4-1, and VH3-48.

In other embodiments, the present invention contemplates a panel of as few as three or four or five antibodies that collectively react with at least 25 to 35% or at least 40% of the variable regions used (i.e. observed in a representative group of cancer patients). For example, in one embodiment, the present invention contemplates a panel of antibodies comprising: a first monoclonal antibody or fragment thereof having immunoreactivity with Ig derived from the VH3-23 family member; a second monoclonal antibody or fragment thereof having immunoreactivity with Ig derived from the VK4-1 family member; a third monoclonal antibody or fragment thereof having immunoreactivity with Ig derived from the VL1 family; and a fourth monoclonal antibody or fragment thereof having immunoreactivity with sIg derived from the VL2 family. In a preferred embodiment, the present invention contemplates a panel comprising a first monoclonal antibody or fragment thereof having immunoreactivity with VH3-23, said first monoclonal antibody being unreactive with VK4-1, VL1, and VL2; a second monoclonal antibody or fragment thereof having immunoreactivity with VK4-1, said second monoclonal antibody being unreactive with VH3-23, VL1, and VL2; a third monoclonal antibody or fragment thereof having immunoreactivity with VL1, said third monoclonal antibody being unreactive with VH3-23, VK4-1, and VL2; and/or a fourth monoclonal antibody or fragment thereof having immunoreactivity with VL2, said fourth monoclonal antibody being unreactive with VH3-23, VK4-1, and VL1.

In certain embodiments, the present invention provides a method to identify at least 10 (more preferably at least 20, and most preferably at least 30) variable region-specific mAbs, as well as the corresponding resulting panel of antibodies. In some embodiments, at least one anti-L_{V1} mAb is identified and included within the panel. In other embodiments, at least one anti-L_{V2} mAb is identified and included within the panel. In further embodiments, at least one anti-K_{V4-1} mAb is identified and included within the panel. In certain embodiments, at least one H_{V3-23} mAb is identified and included within the panel. In a particular embodiment, mAbs against all H_{V}, K_{V}, and L_{V} region families and family members that are highly expressed (i.e. observed with a frequency of 2% or more in a representative group of cancer patients, and more preferably, with a frequency of 1% or more) by the patient population.

In certain embodiments, the present invention provides a database comprising amino acid sequences derived from lymphocyte tumor cell immunoglobulin. In certain embodiments, the database comprises variable region sequences. In another embodiment, the database comprises constant region sequences. In certain embodiments, the variable region comprises a framework epitope for use in designing immunogen. In some embodiments, the variable region sequences comprise Non-Hodgkin's B-Cell Lymphoma sequences. Of course, a variable region sequence may comprise a non-idiotypic variable region specific epitope (e.g framework epitope sequence) unique for a particular patient. For example, this framework epitope sequence may be identified and used to generate antibody and/or to manufacture a patient-specific an immunogenic composition (vaccine).

One method of obtaining framework epitope sequences comprises taking a patient biopsy. In one embodiment, non-idiotypic variable region specific epitopes (e.g., framework epitope sequences) generated from patients diagnosed with a B-cell lymphoma can be used to select the appropriated mAbs from a mAb panel, wherein the biopsy sample may also be used to screen for antibody binding. Alternatively, or in addition, a direct determination of reactivity between a patient's tumor cell, and potentially reactive mAbs could be achieved via immunohistochemical analysis with reagents derived for the variable region-specific mAbs. In certain embodiments, non-antibody-based therapies (e.g. radiation, chemotherapy, etc.) could be used to treat a patient during the preparation of a framework epitope-specific mAb. In a preferred embodiment, the framework epitope-specific mAb (preferably humanized) used for treatment has been previously prepared and the patient can be treated soon after the lymphoma immunoglobulin variable region is characterized, without the need for pre-treatment with conventional therapies. The present invention has the advantage over other anticancer regimens in that variable region-specific mAbs generally only eliminate the variable region-family (or subset) or a family member-specific B-cells, while sparing most of the normal B-cells. Consequently, the patient is less likely to become immunocompromised as a result of the therapy.

In certain embodiments, the present invention provides mAbs directed to the variable region family or family member-specific B-cells, wherein the mAbs have affinity constants that are in the range of currently commercially available therapeutic antibodies (see, e.g., Table 4).

In certain embodiments, the present invention provides an epitope comprising a portion of a framework region located within a variable region. By raising mAbs against framework regions, or framework regions combined with a number of amino acids from a CDR (such as CDR1 or CDR2), the antibody can be prepared in advance and will have a wider spectrum of utility (with a patient population) than antibodies raised to specific CDR regions. While any framework region may be used, in certain embodiments, a framework region epitope is within framework 1 (FR1), wherein FR1 comprises approximately 75 nucleotides (approximately 25 amino acids). FR1 regions, or portions of FR1 regions with one or two amino acids from a CDR, may be used to generate antibodies and/or to manufacture immunogenic compositions. In other embodiments, mAbs are raised against a portion of a CDR region or a full CDR region (which may contain a number of amino acids from a framework region). In certain embodiments, such CDRs or portions of CDRs are CDR1 or CDR2 as these regions are less variable than CDR3.

The exemplary data disclosed herein demonstrate a skewed representation of gene usage with some families and family members being more frequently expressed (see Table 1). In certain embodiments, mAbs generated against HV3-23 recognize at least about 40%, 50% or 60% of the 17% in the patient population (i.e. at least about 6.8%, 8.5% or 10.2% of the patient population). In other embodiments, multiple mAbs are used generated against HV3-23 such that at least 80%, 90% or 100% of the 17% of HV3-23 in the patient population is recognized. In certain embodiments, mAbs generated against KV4-1 recognize at least about 40%, 50%, or 60% of the 12.2% KV4-1 sequences present in a patient population.

In certain embodiments, the present invention provides a mAb panel comprising the minimum number of mAbs to cover between 50-75% of a patient population. In certain embodiments, only four H_{V}-, K_{V}-, or L_{V}-specific mAbs provides the 50% patient population coverage. In certain embodiments, ten H_{V}-, K_{V}-, or L_{V}-specific mAbs provides the 70% patient population coverage. In certain embodiments, the H_{V}-specific mAb may be selected from the group comprising H_{V3-23}, H_{V-3-48}, H_{V3-7}, H_{V3-11}, H_{V3-15}, H_{V3-12}, H_{V3-21}, H_{V3-74}, H_{V4-34}, H_{V4-39}, or H_{V4-59}. In certain embodiments, the LV-specific mAb may be selected from the group comprising L_{V1} or L_{V2}. In certain embodiments, the K_{V}-specific mAb may be selected from the group comprising K_{V4-1}, K_{V1-17}, K_{V1-39}, K_{V1-5}, K_{V2-28}, K_{V2-30}, K_{V3-11}, K_{V3-15}, or K_{V3-10}.

In certain embodiments, the present invention provides a method to measure immunization potency by using variable region-specific mAbs to compare purified framework epitope protein to KLH-conjugated framework epitope protein. In certain embodiments, a strong decrease or loss of immunoreactivity indicates over-conjugation.

In some embodiments, a combinatorial approach is employed with the monoclonal antibodies or antibody fragments of the present invention in both therapeutic and diagnostic applications. As noted above, in order to generate a panel of antibodies or antibody fragments (e.g., for determining what type of variable region a patient expresses on their lymophoma cells), it is generally desirable to have the greatest patient population coverage. One way to achieve this goal is to include members from Table 1 that have a relatively high representation in the patient population. Coverage in any panel that is generated or any therapeutic application, however, can be further increased by targeting both the heavy and light chains of the surface antigenic receptor (e.g. BCR). In other words, since this receptor is composed of two identical heavy chain and two identical light chain variable regions, monoclonal antibodies or fragments thereof directed at either the heavy or light chain variable regions can be used to effectively eradicate the tumor cells or increase the coverage on any antibody panel. By employing antibodies and antibody fragments directed towards both the heavy and light chain of the receptor a combinatorial advantage is gained in terms of building diagnostic panels or compiling a repertoire of mAbs that will treat the largest fraction of the patient population.

While not necessary to understand to practice the present invention, it is believed, mathematically, the average probability that a patient treatment is available, or that a particular panel will successfully determine the type of variable region in a patient sample, can be expressed as follows:
p_{T}=1−(1−p_{H})*(1−p_{L})
Where pT is the fraction of total patients for which an antibody is available, pH and pL are the fraction of patients for which heavy chain and light chain reactive mAbs are available respectively.

By way of example, the HV3-23 and KV4-1 reactive monoclonal antibodies 3C9 and 10H7 react with 47% of the HV3-23 derived heavy chains and 66.7% of the KV4-1 derived light chains respectively as described in the Examples below. Given the percent utilization, 3C9 and 10H7 react with 8.0% and 8.1% of the total patient population respectively. In combination, these two antibodies can have therapeutic activity, or diagnostic coverage in a panel, in 15.5% of the total lymphoma patient population. As another example, consider only the heavy and light chain genes of the BCR (B cell antigen receptors) that are present at 5% or greater in the patient population. For the heavy chain, there are 7 genes representing 60.8% of the total heavy chain utilization. For the light chain, there are 5 genes representing 43.7% of the total light chain utilization. A panel of antibodies directed at this collection of 12 genes would have a hypothetical therapeutic and diagnostic utility in 77.9% of the lymphoma patient population. As another example, consider only the heavy and light chain genes of the BCR that are present at 2% or greater in the patient population. For the heavy chain, there are 13 genes representing 81.8% of the total heavy chain utilization. For the light chain, there are 17 genes representing 80.5% of the total light chain utilization. A panel of antibodies directed at this collection of 30 genes would have a hypothetical therapeutic and diagnostic utility in 96.5% of the lymphoma patient population. As another example, consider only the heavy and light chain genes of the BCR that are present at 1% or greater in the patient population. For the heavy chain, there are 19 genes representing 90.1% of the total heavy chain utilization. For the light chain, there are 26 genes representing 92.5% of the total light chain utilization. A panel of antibodies directed at this collection of 45 genes would have a hypothetical therapeutic and diagnostic utility in 99.3% of the lymphoma patient population. As such, the present invention contemplates such combinatorial approaches for both diagnostic applications (e.g. panels of antibodies) and therapeutic applications (e.g. collection, kit or system containing a particular set of antibodies).

II. Immunotherapy

Passive immunotherapy refers to therapeutic interventions without the direct induction of specific immunity. On the other hand, active immunotherapy refers to the induction of a specific immune response to malignant cells in vivo by an immunization. The present invention contemplates a combination approach comprising both passive and active immunotherapy. In certain embodiments, the present invention provides a method comprising monoclonal antibody (e.g. humanized) passive immunization followed by active immunization (i.e. a vaccine) directed to the same and/or different epitope(s). In a preferred embodiment, the monoclonal antibody is specific for a variable region family member or family epitope and the immunogenic composition comprises a unique tumor-specific idiotype.

Current immunotherapy regimens often take advantage of a prior treatment before any antibody is administered to a patient. The traditional rationale for this approach is that currently utilized immunotherapy is most effective when a tumor burden has been reduced by a previous anticancer treatment. For example, B-cell lymphomas (i.e., for example, Non-Hodgkin's Lymphoma) when initially responsive to cytotoxic chemotherapy, permits application of immunotherapy in the more advantageous setting of minimal residual disease. Timmerman J. M., “Immunotherapy For Lymphomas” Intl J Hematology 77:444-455 (2003).

Two common anticancer treatments are radiotherapy and chemotherapy. Radiotherapy may comprise a localized exposure to a radionuclide source or radioimmunoconjugates (RICs) where antibodies (i.e., for example, monoclonal antibodies) are directly attached to a radioisotope (i.e., for example, ¹³¹I, ⁹⁰Y). RICs may provide targeted radiation therapy but have disadvantages resulting from bystander or crossfire effects. Chemotherapy utilizes multiple dosages and frequent time intervals using such drugs as anthracycline (e.g., deoxyrubicin), fludarabine, etioposide, prednisone, vincristine, cyclophosphamide, or carboplatin/cisplatin. The adverse clinical side effects of chemotherapy are well known in addition to limited clinical success. van de Loosdrecht et al., “Emerging antibody-targeted therapy in leukemia and lymphoma: current concepts and clinical implications” Anti-Cancer Drugs 15:189-201 (2004).

One disadvantage of following any current anticancer therapy (i.e., for example, radiotherapy, chemotherapy, or immunotherapy) is the development of an immunodeficient patient. Consequently, it is usually necessary that any conventional anticancer intervention be ceased for several months before an immunotherapeutic regimen may be initiated. In certain embodiments, the present invention provides a method comprising a patient that has not been previously exposed to any previous anticancer treatment regimen.

In certain embodiments, the combination therapy of the present invention provides methods for treating Non-Hodgkin's Lymphoma in a subject comprising: a) administering to the human a monoclonal antibody, or fragment thereof, reactive with a non-idiotypic variable region framework epitope of an immunoglobulin determined to be present on the subjects Non-Hodgkin's Lymphoma; and b) immunizing the subject with at least a portion of the immunoglobulin present on the subject's Non-Hodgkin's Lymphoma.

One example of combination therapy is as follows. Patients diagnosed with a B cell disorder wherein the tumorigenic B cell continues to express the B cell antigen receptor, for example follicular Non-Hodgkin's Lymphoma, are candidates for combination therapy using a non-idiotypic variable region specific monoclonal initially, followed by personalized idiotypic protein vaccine treatment. Patient biopsy tissue is used for RNA isolation and determination of the patient tumor derived variable region idiotype protein sequence. Coincident with this or immediately following, mounted biopsy sections, either frozen or paraffin embedded, are used along with a diagnostic panel of non-idiotypic variable region specific monoclonal antibodies to confirm that the therapeutic monoclonal antibody is available for patient treatment. This confirmation is achieved upon successfully demonstrating that an antibody in the diagnostic panel binds to the tumor cells' surface. This identifies the therapeutic antibody to be given to the patient. Administration of monoclonal is done by infusion based on dosing requirements suitable for the patient's size. Reduction of tumor volume can be monitored by CT scans, tumor specific protein or nucleic acid assays, or other means. Monoclonal antibody infusion may be discontinued prior to vaccination with idiotypic protein vaccine. ELISA assays can be used to determine the residual level of non-idiotypic variable region specific monoclonal in patient serum. In certain embodiments, a complex of the non-idiotypic variable region specific mAb and vaccine protein is employed. Upon determination that the non-idiotypic variable region specific monoclonal has achieved the appropriate level of tumor reduction, administration of the idiotype vaccine can proceed. Briefly KLH conjugated idiotype protein is administered via subcutaneous injection. Typicall, a co-local injection of the GM-CSF adjuvant is also administered. The vaccination regime may include, for example about 5 to 16 to 30 vaccinations (or additional vaccinations), such vaccinations given, for example, over the course of time from less than one month to more than one year.

A. Passive Immunothereapy

In certain embodiments, a patient is administered a non-idiotypic variable region specific mAb or fragment thereof that is specific for the family or family member variable region determined to be expressed by the patient's B lymphoma cells. The patient is administered a composition comprising a sufficient quantity of this mAb or fragment thereof to a least partially reduce the tumor load in the patient.

The non-idiotypic variable region specific mAb or fragments for passive immunotherapy may be produced and purified using any type of methods. Such production and purification methods are known in the art. One particular example of such production and purification, for large scale production, is as follows. DNA plasmid vectors containing coding sequences for heavy and light chain mouse-human chimeric genes and the dhihydrofolate reductase (DHFR) gene are constructed. The DNA mixture is electroporated into Chinese Hamster Ovary cells that are deficient in DHFR expression. After recovery, the cells are plated in growth medium that does not contain thymidine, glycine, or hypoxanthine for selection of cells that have incorporated the DHFR encoding vectors as well as the heavy and light chain DNA. Cells that survive the selection are expanded and then exposed to low levels of methotrexate in the medium, which is an inhibitor of DHFR and allows the selection of cells that have become resistant to the inhibitor by amplification of the integrated DHFR genes. Upon adequate expansion of the cells, cell supernatant is assayed for the concentration of secreted monoclonal antibody using an ELISA method for the detection of immunoglobulin. In brief, microtiter plates are coated with anti heavy chain specific antibodies. After blocking of the plate, diluted supernatant from the recombinant CHO cells is allowed to react with the coated plates. After washing away excess supernatant, bound recombinant antibodies are detected by first binding biotinylated anti light chain reactive antibodies followed by HRP-conjugated streptavidin. After washing, TMB substrate is added and allowed to develop. Clones of CHO cells demonstrating high production levels of monoclonal antibody are selected for additional rounds of growth in increasingly higher concentrations of methotrexate in order to bring about coordinate gene amplification that results in an increased specific productivity of the cells producing monoclonal antibody. For large scale production, the development of the CHO cell line also includes the adaptation of the cells for suspension growth in serum and animal protein-free media. Selection of the production cell line continues until a productivity target of at least 150 mg of protein per liter of cells is achieved. Upon successful completion of cell line development, the cell line is re-cloned as necessary, tested for the presence of adventitious agents including virus, and further characterized for stability of protein production. Aliquots of the cells are frozen to serve as a Master Cell Bank. For a production run, an aliquot of the Master Cell Bank is thawed and the cells are expanded into increasingly larger growth vessels until a sufficient quantity of cells has been generated for inoculating a production bioreactor. Upon completion of the bioreactor culture, cell debris is separated from the crude harvest supernatant. Secreted monoclonal antibody is then captured by affinity chromatography on a Staphyloccocus aureus Protein A column for isolation of crude monoclonal product. The Protein A affinity-purified pool is then further purified on an ion exchange column. The final purified monoclonal is then sterile purified using a 0.2 micron filter. Material is diafiltered into the final formulation buffer and diluted in this buffer to a final concentration of 20 mg/ml. 20 ml (400 mg) aliquots are aseptically filled into sterile glass vials that are stoppered and crimp-sealed.

Another example for determining the antibody that is reactive with a particular patient lymphoma is as follows. First, a biopsy sample is obtained from the patient. This sample will typically be rendered in the form of the frozen or paraffin embedded tissue section. Monoclonal antibodies are generated using a hybridoma or recombinant cell production cell line process. In brief, recombinant cells are prepared using recombinant DNA vectors are derive from the specificity determining variable region heavy and light chains combined with the desired heavy and light chain constant regions. The heavy and light chain vectors with the appropriate promoter and enhancer sequences in selectable markers are used to stably transform mammalian cell lines. After selection for recombinant protein production and further amplification of the recombinant protein production level, these recombinant cells are ready for production level processes. Hybridoma or recombinant cells are seeded into a large format protein production vessel for manufacturing of the recombinant monoclonal antibody. After purification monoclonal antibodies are biotinylated. These reagents are ready for interaction with frozen or paraffin embedded tissue sections. Microscope slide mounted tissue sections are incubated with each biotinylated non-idiotypic variable region reactive antibody. Specifically bound antibody is then visualized by incubation with a streptavidin-horseradish peroxidase conjugate followed by incubation with the peroxidase substrate diaminobenzidine. Positive staining is observed as a brown precipitate. Sections are then counterstained with hematoxylin, which stains nuclei blue. Visualization of tumor cells where the non-idiotypic variable region reactive monoclonal antibody has stained the cells provides the necessary evidence that the tumor is expressing a surface antigen that is reactive with a particular monoclonal. In general this assay will also identify the particular variable region used by the tumor surface antigen.

Another example that may be used to identify the appropriate monoclonal antibody for passive therapy involves a pooled strategy for detection of binding with immunohistochemistry. Biotinylated monoclonal antibodies with non-idiotypic variable region reactivity are prepared. Biotinylated monoclonal antibodies with non-idiotypic variable region reactivity are prepared as described above. Mounted frozen or paraffin embedded tissue sections are obtained from patient biopsies. Multiple different monoclonal antibodies are formed into mixtures such that each mixture has specificity against multiple different variable region gene sequences that can be expressed on the tumor cells surface. Although a mixture may contain as few as two non-idiotypic variable region reactive monoclonals, it is envisioned that mixtures of four or eight monoclonals will typically be used. After washing away unreacted antibody, the tissue sections are incubated with horseradish peroxidase-conjugated streptavidin reagent followed by incubation in the peroxidase substrate diaminobenzidine. Upon visualization it is determined whether the tumor cells have been stained with one or more of the antibodies contained within a particular mixture. Upon determination that a particular mixture has reactivity subsequent determination of the particular monoclonal or monoclonals that are reactive with the tumor can be accomplished as demonstrated in the example above. In this way, a smaller number of tissue sections will be required.

Another example that may be used to identify the appropriate monoclonal antibody for passive therapy involves a multiple labeled strategy for detecting of binding with immunohistochemistry. Purified monoclonal antibodies with non-idiotypic variable region reactivity are prepared as described above. The collection of monoclonals is divided into groups with defined reactivity. Within a group each of the monoclonal antibodies is conjugated so as to provide means for distinction in visualization on tissue binding as compared with other members of the group. For example, in a group containing a pair of antibodies, one antibody can be conjugated to horseradish peroxidase while another is conjugated to alkaline phosphatase. The substrate mixture for development would include for example DAB and Fast Red which yield distinctly colored staining upon enzymatic reaction with these enzymes. Alternatively, a fluorescent detection scheme can be employed using for example the fluorophores AMCA, FITC, Cy3 and Cy5. Alternately, groups can be prepared with antibodies conjugated to fluorescent beads where many more spectral combinations are possible. Mounted frozen or paraffin embedded tissue sections are obtained from tissue biopsies. The group of labeled non-idiotypic variable region reactive monoclonal antibodies is allowed to react in phosphate buffered saline with the tissue sample. After washing away non-reacted excess antibody, the slide section can be visualized directly for chromatogenic enzymatic reaction products or by using fluorescence microscopy. Determination of which specific non-idiotypic variable region reactive monoclonal binds with the patient tumor cells can thus be obtained.

A database of binding reactivity for each of the monoclonal non-idiotypic variable region reactive antibodies is generated where the sequences of the variable regions are associated with the degree to which a particular monoclonal has shown binding reactivity. Typically the monoclonals will only react within a particular gene family member, however all known reactivity will be recorded in the database. The binding data for the database is determined via ELISA where a particular monoclonal is assayed against multiple different protein sequences covering a broad range of sequence possibilities. Inspection of this database allows for certain patterns of binding to be easily characterized. Advanced analysis of sequence differences allows for even greater detail in predicting whether a monoclonal will bind given only the primary protein variable region sequence. Patient biopsy tissue samples are obtained as both mounted sections and material to be homogenized and used for nucleic acid extraction. Determination of tumor gene utilization is performed as described above. Patient tumor variable region sequences are compared to database sequences using algorithms to characterize sequence similarity. Advanced analyses will focus especially within the regions that affect binding of particular monoclonals. Based on this analysis, one or more monoclonal non-idiotypic variable region reactive antibodies are selected for further characterization in immunohistochemical assays as described above.

Another example that may be used to identify the appropriate monoclonal antibody for passive therapy involves FACS analysis of the tumor sample. Labeled non-idiotypic variable region reactive antibodies are prepared for FACs analysis as described above. A database of the normal tissue distribution of the binding reactivity of these antibodies is prepared by analysis of data from FACS studies of binding to B cell populations. This database will contain the percentage of B cells that are reactive to particular panel antibodies, for example, anti-IGHV3-23 monoclonal 3C9. Typing of tumor sample can be achieved, for example, by analysis of binding of patient peripheral blood samples with the panel of monoclonal antibodies. A significant increase in the percentage of B cells stained by a particular monoclonal antibody will indicate a clonal expansion of a particular B cell line consistent with lymphoma. Analysis for additional markers of lymphoma that correlate with staining by a non-idiotypic variable region specific panel antibody can be performed to specifically select one or more panel antibodies for therapy.

In certain embodiments, the FACS analysis is performed as follows. Purified monoclonal antibodies are prepared using a hybridoma or recombinant cell line process. Antibodies are conjugated to various fluorophores (eg. fluorescein iso-thiocyanate, phycoerythrin, allophycocyanin, peridinin chlorophyll protein) or reactive markers (e.g. biotin) to create labeled monoclonal antibodies that are reactive with specific subpopulations of B cells based on the expressed variable region gene (see, e.g., Table 1). Peripheral blood cells are prepared for fluorescence activated cell scanning or sorting (FACS) using conventional methods. Briefly, a cell suspension is incubated with one or more monoclonal antibodies directed at cellular protein targets. These antibodies include reactivity against common cell surface antigens (eg. CD20, BCR constant regions) as well as mixtures containing one or more uniquely labeled non-idiotypic variable region antibodies to prepare labeled cell suspensions. The cell suspension is analyzed using commercially available instruments (eg, FACSCalibur) where information on the quantity and correlation of markers labeled by specific monoclonal antibodies is obtained. For example, it is determined that a B cell expresses a surface antigen receptor derived from the IGHV3-23 gene family, when correlation in labeling with anti-CD20 and anti-IGHV3-23 antibodies is observed. In other embodiments, monoclonal antibodies specific for human heavy a light chain families may be used to enumerate, characterize, and/or isolate cells normal or diseased B cells using standard flow cytometry techniques. Briefly, B cells expressing on their surface an immunoglobulin belonging to a particular light or heavy chain family can easily identified in a mixed cellular population by incubating these cells with a fluorescently-labeled form of the cognate monoclonal antibody and detecting the cell-associated fluorescence using a flow cytometer. In addition, these antibody-labeled B cells can be characterized further by including additional monoclonal antibodies specific for surface markers of interest such as CD20, CD19, CD23, and CD5. Finally, antibody-labeled B cells can be isolated by using a sorting flow cytometer that physically segregates cell population based on user-defined patterns of cell-associated fluorescence.

In certain embodiments, the dosage and suitability of this treatment is determined with a pre-screening step to quantitate the non-idiotypic variable region-specific mAb binding to circulating immonoglobulin in the patient's serum. Since patients will have normal and tumor soluble immunoglobulin in serum that will bind the non-idiotype V region-specific mAb, in certain embodiments, these levels are determined prior to treatment. In addition to preventing the uptake of immunotherapy by tumor cells, excessive cross-reactive normal and/or tumor binding may result in the patients inability to clear these antigen:antibody complex formation. The resulting levels that are determined may, for example, be used to calculate the amount of mAb to be administered to the patient. In some embodiments, plasmapheresis or similar methods are performed on patients to lower serum levels of tumor-related V regions. These assays measure the normal and tumor soluble V region levels in serum that are recognized by the non-idiotype V region-specific mAb selected from the panel of mAb shown to stain patient tumor. In particular embodiments, prior to administering a non-idiotype V region-specific mAb patient serum would be tested for serum levels of immunoreactive with the selected mAb.

One particular method of measuring tumor and tumor-related V region levels in patient serum involves using a sandwich or capture ELISA using the following exemplary protocol. Patient serum samples, normal pooled human serum, or a purified Id protein known to be immunoreactive are serially diluted in PBS with 5% BSA in a 96-well microtiter plate previously coated with the potential therapeutic non-idiotype V region-specific mAb. Either HRP-conjugated-goat-anti-human IgG or biotinylated-non-idiotype V region-specific mAb can be used to detect binding of patient V region to non-idiotype V region-specific mAb. An additional incubation with HRP conjugated-streptavidin is added when using biotinylated-non-idiotype V region-specific mAb. The presence of HRP is measured with the addition of substrate solution. The known immunoreactive purified Id protein is used to prepare a standard curve for quantitative measuring of immunoreactive Ig level in the patient serum and the normal pooled human serum is a control.

Another method for measuring tumor and tumor-related V region levels in patient serum involves the use of an inhibition ELISA using the following exemplary protocol. Purified Id protein known to bind the potential therapeutic mAb is coated to a 96-well microtiter plate. A fixed dilution of patient serum is pre-incubated with a serial dilution of the non-idiotype V region-specific mAb being tested and a standard curve is generated using the serial dilutions of the same Id protein coupled to the microliter plate or a second Id protein know to bind the mAb. This incubation is performed prior to adding the samples to the Id protein coated plate. HRP-conjugated-goat-anti-mouse IgG is used to detect binding of unbound mAb to the pre-coated Id protein. The presence of HRP is measured with the addition of substrate solution. Reactivity of patient serum is compared with that of purified Id protein concentrations known to bind the mAb. The normal pooled human serum is used as a control.

B. Active Immunotherapy

In certain embodiments, a patient is treated with immunogenic compositions to induce the patient's immune system to produce a specific immune response to a malignancy. In some preferred embodiments, the immunogenic composition used in active immunotherapy comprises one or more antigens derived from a patient malignant cells. In some particularly preferred embodiments, the immunogenic composition comprises at least an idiotypic portion of an immunoglobulin derived from a subject's own malignant cell(s). For example, B-cell lymphoma cells have on their surface particular immunoglobulins. These immunoglobulins, particularly the idiotypic portions (“idiotypic proteins”) can be used as antigens in immunogenic compositions to produce patient-specific idiotypic vaccines. In certain embodiments, the idiotypic proteins are produced recombinantly. In some embodiments, particular individual recombinant idiotypic proteins are selected for use, while in other embodiments, multiple, tumor-specific idiotypic proteins are used in a multivalent composition (see, e.g., U.S. Pat. No. 5,972,334 to Denney, issued Oct. 25, 1999, incorporated by reference herein in its entirety). In certain embodiments, the idiotypic protein is a recombinant idiotype (Id) immunoglobulin (Ig) derived from a patient's B-cell lymphoma [IgG₃ with either a kappa (κ) or a lambda (λ) light chain] obtained from each patient, e.g., as described in Example 1. In preferred embodiments, the immunogenic composition comprises the same heavy and light chain V region sequences expressed by the patient's tumor.

In certain embodiments, the idiotypic protein is conjugated to a carrier, e.g., a protein using techniques which are well-known in the art. Materials that are commonly chemically coupled to the antigens e.g., to enhance antigenicity, include keyhole limpet hemocyanin (KLH), thyroglobulin (THY), bovine serum albumin (BSA), ovalbumin (OVA), tetanus toxoid (TT), diphtheria toxoid, and tuberculin purified protein derivative. In preferred embodiments, KLH manufactured under cGMP conditions is obtained from biosyn Arzneimittel GmbH and used for the preparation of Id-KLH conjugates.

In some embodiments, a cytokine is linked to the idiotypic protein. In certain embodiment, the immunogenic composition produced comprises a fusion protein comprising the idiotypic protein and a cytokine such as GM-CSF, IL-2 or IL-4 (see, e.g., PCT International Application PCT/US93/09895, Publication No. WO 94/08601 and Tao and Levy (1993) Nature 362:755 and Chen et al. (1994) J. Immunol. 153:4775; all of which are herein incorporated by reference). Generally in such fusion proteins, sequences encoding the desired cytokine are added to the 3′ end of sequences encoding the idiotypic protein.

Exemplary Production Methods

General methods of producing patient-specific immunogenic compositions are exemplified by the production of KLH-conjugated autologous immunoglobulin (idiotypic) protein. Production and use of this composition for active immunotherapy is provided by way of example and is not intended as a limitation (for example, production of immunogenic compositions may comprise different cloning methods, different proteins produced, different carriers linked by other conjugation or fusing methods known in the art, etc.)

The production of a patient-specific immunogenic composition can be described as having the following stages: cloning of the gene or genes that encode a particular antigen protein in a patient's tumor cells; generation of amplified cell lines expressing a recombinant version of the antigen protein expressed by the patient's tumor; expansion of the amplified cell line; and purification of the recombinant antigen protein.

In certain embodiments, the purified protein is conjugated (e.g., to KLH) prior to packaging (e.g., filling and vialing) of the final biological product. By way of example and not by way of limitation, one method of producing KLH-conjugated autologous immunoglobulin (idiotypic) protein comprises: 1) cloning of the variable region genes for the heavy and light chains expressed in a patient's tumor; 2) generation of amplified cell lines expressing a recombinant immunoglobulin (Ig) molecule comprising the heavy and light chain variable regions expressed by the patient's tumor; 3) expansion of the amplified cell line; 4) purification of the recombinant Ig molecule; 5) conjugation of the purified Ig to KLH; and 6) filling and vialing the final biological product. As noted above, in some embodiments, multiple different antigenic proteins expressed by a patient's tumor are produced, while in other embodiments, individual antigenic proteins are selected for expression to make the final product.

Tumor Samples

The initial step in producing an autologous immunotherapy is the acquisition of tumor cells from the patient. For example, for a B-cell lymphoma patient, suitable tumor samples may be obtained, e.g., by surgical biopsy of an enlarged lymph node (LN) or other extranodal tissue involved by lymphoma, by fine needle aspiration (FNA) of an enlarged LN, by phlebotomy or aspirate of a patient whose blood or other fluids contains greater than about 5×10⁶ lymphoma cells/mL (quantitated by manual differential); or 4) bone marrow (BM) aspiration when the patient's BM contains greater than about 30% involvement (percentage of total inter-trabecular space). It is contemplated that each patient is assigned a patient identification number (PIN) for identification purposes and all samples are labeled with this PIN.

Cloning Genes Expressing Immunogenic Proteins

Cloning of genes encoding immunogenic proteins expressed in the sampled tumor cells may be accomplished by standard molecular biological techniques. For example, cloning from RNA (e.g., mRNAs transcribed from genes of interest, such as rearranged immunoglobulin genes) generally comprises reverse transcription to produce a cDNA, and may also comprise amplification (e.g., by polymerase chain reaction). In some embodiments, amplification comprises the use of primers that specifically amplify the gene segment of interest. In other embodiments, amplification comprises the use of primers that will co-amplify multiple, different gene segments (e.g., will amplify most or all members of particular family of genes at the same time using, e.g., regions of sequence that are conserved such that a single primer pair will amplify multiple target sequences, or using a mixture of primer pairs in a single reaction).

It is contemplated that amplification products may be purified prior to cloning. For example, the nucleic acid products of PCR amplification can be purified using methods known in the art. In certain embodiments, amplification products are precipitated, e.g., using alcohol such as ethanol or isopropanol. In other embodiments, the amplification products are purified using kits made for that purpose (e.g., the QIAquick PCR Purification Kit, Quiagen GmbH, Hilden Germany). In certain embodiments, amplification products are resolved by electrophoresis, e.g., in agarose, and particular products are excised from the gel and purified, e.g., using a process such as that provided by the QIAquick Gel Extraction kit (Quiagen GmbH, Hilden, Germany). It is contemplated that purification methods may be used alone or in combination.

Tumor derived gene products, such as the amplification products described above, are then cloned into an expression vector. Amplification products may be individually cloned (e.g., one amplification product combined with one vector) or they may be combined with other products prior to cloning. By way of example and not by way of limitation, Example 1 below describes the cloning of tumor-associated idiotypic proteins from Non-Hodgkin's B Cell lymphoma patients. In the process described in Example 1, combinations of amplification products from reactions using two different anchor primers each separately used in combination with one of a set of five different constant region primers. Products of amplifications that used the same constant region primer (but different anchor primers) were combined prior to purification and ligation into an expression vector. Following transformation into E. Coli and growth on selective medium, transformants were screened by PCR for each of the five constant chains.

It is contemplated that the cloned sequences will be analyzed to determine their association with a patient's tumor, or to determine additional information about a gene cloned from a tumor. By way of example and not by way of limitation, the clones may be analyzed by any of the methods known in the art for detecting or characterizing nucleic acids based on the sequences they contain, including but not limited to DNA sequencing, probe hybridization, PCR, etc. See, e.g., Example 1, which describes the characterization of cloned tumor-derived variable region genes by DNA sequencing individual cloned genes.

Preparation and Linearization of Expression Vector DNA

When a clone comprising the tumor-derived gene has been identified, a large-scale plasmid preparation is made for use in the generation of stable cell lines expressing the patient's tumor-derived antigen protein. Generation of such stable cell lines comprises transfection of a host cell with one or more expression vectors encoding the tumor-specific antigen proteins. In certain embodiments, combinations of clones will be used together. For example, in certain embodiments a pair of expression vectors is identified that contains the same heavy and light chain variable region immunoglobulin sequences expressed by the patient's tumor. Large-scale preparations of each of the plasmids to be used together are made.

Plasmid DNA is isolated using standard methods known in the art. For example, in some embodiments, plasmid DNA is isolated using alkaline lysis followed by purified by ion exchange chromatography using a plasmid isolation kit (QUIAGEN, Inc., Valencia, Calif.).

In certain embodiments, the expression vectors containing the insert sequences are linearized prior to the transfection into a host cell. In preferred embodiments, the vectors linearized by digestion with a restriction enzyme that cuts only once within the plasmid backbone. In certain embodiments, multiple expression vectors comprising insert sequences are used together and each expression vector is used. For example, when a pair of expression vectors containing the same heavy and light chain V region sequences expressed by the patient's tumor are to be used, the heavy and light chain expression vectors are linearized as described above. In certain embodiments, to confirm that the desired expression vectors are linearized, an aliquot of DNA is withdrawn following the linearization reaction, the DNA is electrophoresed to confirm linearization, and the sequence of the cloned gene(s) is obtained by DNA sequencing.

Expression vectors, e.g., the linearized expression vectors described above, are then transfected into host cells. It is contemplated that transfection may be by any of the methods known in the art. For example, transfection may be accomplished by the use of carrier molecules, such as DEAE-dextran, or by the use of delivery vehicles such as liposomes and phage particles. In some embodiments, host cells are transfected with the linearized expression vectors using electroporation, while in other embodiments, host cells are transfected by bombardment with nucleic-acid-coated carrier particles (gene gun), or by microinjection.

Host Cells and the Cell Bank System

In certain embodiments, a eukaryotic cell bank system comprising a master cell bank (MCB) and a working cell bank (WCB) is generated. In certain embodiments, a T-lymphoid cell line is employed. In preferred embodiments, the mouse T cell line BW5147.G.1.4 (ATCC TIB 48) is employed.

Generation of a Master Cell Bank and Working Cell Lines

A vial of frozen cells is obtained. It is not necessary to determine the passage number at the time of receipt not to record or determine the number of passages prior to generation of the Master Cell Bank. The cells are expanded, e.g., in a medium such as RPMI 1640 medium containing 10% fetal bovine serum. The cells are then collected, e.g., by centrifugation, and resuspended in 90% fetal bovine serum, 10% DMSO and dispensed into cyrovials (at about 2.16×10⁶ cells/vial) to form the MCB. The cryovials are placed at ≦70° C., then transferred to the vapor phase of a liquid nitrogen freezer for long term storage.

The cells from the MCB vial are expanded and passaged additional times, e.g., three times. The WCB is cryopreserved, e.g., at passage four, in 90% fetal bovine serum, 10% DMSO and dispensed into cryovials, containing an average of about 8-12×10⁶ cells /vial (e.g., 9.59×10⁶ cells/vial) at a viability of about 91%. The cryovials are placed at ≦70° C. and then transferred to the vapor phase of a liquid nitrogen freezer for long term storage. All vials are stored in the vapor phase of a liquid nitrogen freezer.

In certain embodiments, a vial of frozen cells from the MCB is transferred to a service lab such as BioReliance Corporation, Rockville, Md. (now Invitrogen Corporation, Carlsbad, Calif.) to generate a WCB and characterize it with respect to the number of cells per vial and the viability.

A Working Cell Line is generated by thawing a vial of cryopreserved cells from the WCB and culturing the cells, e.g., in RPMI 1640 medium containing 10% fetal bovine serum. The culture generated from the thawed cells is marked as passage 1. A working cell line is split every 2-3 days at a dilution of up to 1:50. A new working cell line is generated when or before the existing working cell line has reached the fiftieth (50th) passage.

Transfection

The linearized expression vector(s) encoding tumor-derived antigen proteins are transfected into host cells e.g., cells from the working cell line (e.g., BW5147.G.1.4 cells). In certain embodiments, the linearized expression vectors are co-transfected along with an expression vector encoding hypoxanthine phosphoribosyltransferase (HPRT; pMSD4-HPRT or comparable) and an expression vector encoding dihydrofolate reductase (DHFR; pSSD7-DHFR or comparable). In preferred embodiments, these expression vectors are also linearized prior to transfection. The HPRT and DHFR expression vectors permit the selection and amplification of cells containing the transfected DNA, including the cells that contain expression vectors encoding tumor-derived antigen proteins.

Generation of Stable Cell Lines Expressing Recombinant Tumor-Derived Proteins

Following transfection as described above, the transfected cells are cultered, e.g., in RPMI 1640 medium containing fetal bovine serum (FBS), non-essential amino acids, sodium pyruvate, L-glutamine and Gentamicin, for 18 to 30 hours. The transfected cells are then plated in multi-well tissue culture plates at appropriate dilutions in medium that requires HPRT expression (“selection medium”). Selection medium does not contain any antibiotic and no antibiotics are used in cell culture media from this stage onward. In certain embodiments, less than one molecule of gentamicin or one molar equivalent of gentamicin sulfate would be present in a dose of a final product (e.g., a carrier conjugated tumor-derived protein) assuming the minimum dilution that a cell line is subjected to from electroporation through final expansion followed by purification of the protein and formulation of the conjugate.

In preferred embodiments, cells that have stably integrated the transfected DNA will grow in selection medium while cells that have not integrated the transfected DNA will be killed. In certain embodiments, approximately three weeks after plating in selection medium, cell culture supernatant is harvested from the transfected colonies (“primary colonies”) and screened for production of the tumor-derived antigen protein, e.g., by ELISA. Primary colonies that express a sufficiently high level of the protein are switched from serum-containing medium to serum-free medium (e.g., HyQ CCM1).

If the tumor-derived protein expression level in the primary colonies is not sufficiently high, primary colonies expressing a range of levels are expanded and plated in medium containing a fixed range of increasing concentrations of methotrexate to permit the identification of colonies containing amplified amounts of the integrated DNA. Cell culture supernatants are assayed for tumor-derived antigen protein production (e.g., by ELISA) at each round of amplification to identify clones that have coordinately amplified the tumor-derived protein expression vector(s) and the DHFR expression vector. Amplification is continued until one or more amplified cell lines expressing a sufficiently high level of recombinant tumor protein is generated.

Once candidate lines expressing sufficiently high levels of expression are identified, aliquots of the cell culture are removed for cryopreservation back-up cultures. In certain embodiments, an expressing cell line (the “production cell line”) is switched from growth in serum-containing medium to growth in serum-free medium (HyQ CCM1). The cell line is generally tested for the presence of mycoplasma, e.g., by PCR methodology or using methodology generally used in the characterization of cell lines used to produce biologicals.

In certain embodiments, a sample is taken for bioburden or sterility testing, and the production cell line is expanded into a closed system comprising a gas-permeable cell culture bag containing a volume of HyQ CCM1 medium HyClone Laboratories, Inc. (Logan, Utah) sufficient to yield an initial seed density of about 5×10⁴ to 5×10⁵ cells/mL. Generally, upon thawing, the medium is checked to ensure it is free of precipitation or turbidity and that it is within the correct pH range as judged by the color of the phenol red in the medium. Generally, thawed CCM1 media is processed over a Protein G column, and the flow-through is filtered through a 0.2 μm filter prior to use.

The production cell line is expanded, e.g., in closed cell culture bags, using a medium such as the processed HyQ CCM1 as the growth medium until the desired volume of cell culture is generated. The necessary volume depends on the tumor-derived protein expression level.

Verification of Expression of Correct Genes in Amplified Cell Lines

In certain embodiments, the identity of the tumor-derived protein(s) expressed in the production cell line is verified by isolating RNA from a sample removed from the gas-permeable cell culture bag. Following isolation of RNA, cDNA is generated and sequenced. Comparison of the sequences obtained from cDNA derived from the amplified cell line and from cDNA derived from the patient's tumor confirms the identity of the recombinant protein secreted by the amplified cell line.

Alternatively, the tumor-derived genes integrated in the production cell line may be verified by PCR amplification of the gene sequence from DNA recovered from cells after harvest of the protein, followed, e.g., by DNA sequencing of the PCR product.

Filtration Harvest of Cell Culture Supernatant

In certain embodiments, once the production line has been expanded into the desired final volume, the cells are grown for at least 8 days. The culture broth is clarified by passing through a filter assembly. In preferred embodiments, the filter assembly comprises three filters in series (a 6 μm, a 0.2 μm and a 50 nm viral filter) (Pall Corporation). Prior to use, the 0.2 μm filter is sterilized by either autoclaving or gamma-irradiation.

Immediately prior to passing the culture through the filter assembly, aliquots are removed for mycoplasma, gene sequence, and adventitious agents testing. Adventitious agent testing is accomplished, e.g., by adding cell lysates to Vero cells growing in cell culture and examining the Vero cells over 14 days for cytopathic effects. The Vero cultures are also tested for hemadsorption.

While not limiting the present invention to any particular method of filtration, generally, filtration is accomplished by attaching the tissue culture bags containing cultures to be harvested to a filter assembly as described above. The filtered supernatant is collected into a sterile container or reservoir bag that is in line with the terminal filter in the series. Once the filtered supernatant is collected into the container or reservoir bag, the container or bag is sealed. The filtered supernatant is then additionally purified.

Purification of Recombinant Tumor-Derived Protein

In certain embodiments in which the tumor-derived proteins are immunoglobulins (e.g., Id proteins), the recombinant proteins are purified from the filtered supernatant by affinity chromatography, e.g., using single-use columns such as 5 mL Protein G columns (GE Healthcare, Piscataway, N.J.). The Protein G resin comprises a recombinant Protein G molecule that lacks albumin binding sites. To purify the recombinant Id protein, the Protein G column is equilibrated with phosphate buffered saline, pH 7.0 (PBS) (Mediatech). The reservoir bag containing the filtered supernatant is connected to the inlet line of the chromatography system. The supernatant is pumped through the Protein G column and the flow through is collected. The column is washed with PBS until the OD₂₈₀ is less than 0.05 (about 100 mL). The bound protein is eluted, e.g., with 0.1 M glycine, pH 2.7. The protein concentration after elution may be adjusted by dilution, e.g., with 0.1 M glycine, pH 2.7. The eluted Id protein is generally incubated at pH 2.7 at room temperature for a minimum of about 30 minutes to inactivate virus. In certain embodiments, the eluted protein is dialyzed, e.g., against 0.9% sodium chloride, USP (Abbott Laboratories, North Chicago, Ill.), to remove the glycine. In some cases, dialysis of the eluted protein is performed against a solution having a lower concentration of sodium chloride and lower pH to enhance the solubility of the Id protein.

In certain embodiments, following dialysis, the purified Id protein is filtered through a 0.2 μm filter. An aliquot is removed and the concentration of the Id protein preparation is determined by measuring the absorbance at 280 nm (OD₂₈₀). If the concentration of the purified Id protein is <0.5 mg/mL the sample may be concentrated by ultracentrifugation, e.g., using a Centriplus® Centrifugal Filter Device (Millipore Corporation, Bedford, Mass.) or an equivalent single-use concentration device. In other embodiments, the Id protein solution is concentrated by ultracentrifugation prior to filtration through the 0.2 μm filter to avoid the need for two filtration steps. Once a concentration of ≧0.5 mg/mL is achieved, the Id solution is filtered through a 0.2 μm filter. Alternatively, the lot may be combined with another lot(s) of purified Id at higher concentration(s) to achieve an average concentration ≧0.5 mg/mL. The protein concentration may be adjusted by dilution with 0.9% sodium chloride, USP. Generally, the product is filtered and additional aliquots are removed and tested for sterility and purity.

The purity of an Id protein preparation may be determined, e.g., by SDS-PAGE. In certain embodiments, Id proteins are applied to a gel, such as a pre-cast gradient polyacrylamide gel (Invitrogen Corp., Carlsbad, Calif.). The proteins are generally applied in a sample buffer with and without a reducing agent and SDS. SDS running buffer is employed in electrophoresis. Broad range molecular weight protein markers (200-6.5 kD) and a reference Id protein are applied to the gel as marker proteins. Following electrophoresis, the gel is stained, e.g., with Coomassie blue stain, and purity is determined. These electrophoretic conditions will display the heavy and light chains of an Id protein and will permit the detection of contaminating protein species of higher and lower molecular weight than the heavy and light chains.

An aliquot of the Id protein preparation is removed and aliquotted for use in in vitro immune response assays. The vials are stored at ≦20° C. The remaining purified Id protein is then processed for final formulation or stored at ≦20° C. prior to final formulation. There are two potential reprocessing steps in the production process: 1) refiltration of the filtered culture supernatant over a new DV50 virus filter which could be used should the initial filter fail the post-use integrity test and 2) refiltration of the filtered purified Id over another 0.2 μm filter.

Those skilled in the art will appreciate that equivalent purification and characterization methods are known and can be applied to non-immunoglobulin proteins expressed according to the methods of the present invention.

Container and Closure System

In certain embodiments, following dialysis and filtration, purified tumor-derived protein, e.g., Id protein, is placed in a sterile container such as a sterile polypropylene tube. Aliquots of unconjugated Id protein may also be removed for the purpose of immune response testing and stored, e.g., at ≦20° C. in sterile pyrogen-free polypropylene vials. In certain embodiments, the remaining purified protein is then processed for final formulation or stored at <20° C. prior to final formulation.

Preparation of Drug Product

In certain embodiments, the final biologic product comprises purified tumor-derived antigen protein conjugated or fused to a carrier. In preferred embodiments, the purified protein is conjugated or fused to KLH. By way of example, and not by way of limitation, one example of a final biologic product is a composition composed of a 1 mL solution for subcutaneous injection containing: 1) Recombinant Id-KLH Conjugate at 1.0 mg, and 2) 0.9% Sodium Chloride, USP at 1.0 mL. The final biologic product, the recombinant Id-KLH conjugate, is manufactured by chemically coupling KLH to the purified recombinant Id protein (the biologic substance) using glutaraldehyde. KLH (VACMUN® liquid) is obtained, e.g., from biosyn Arzneimittel GmbH (Fellbach, Germany). In preferred embodiments, KLH manufactured under cGMP conditions is used.

The conjugation reaction is carried out by mixing equal amounts by weight of purified recombinant protein such as an Id protein and KLH in a disposable, sterile, pyrogen-free polypropylene tube. In certain embodiments, mixing is performed in a Class 100 BSC. Depending on the expression level of a patient's cell line, multiple Protein G eluates (purified Id) may be pooled to yield sufficient Id protein. Glutaraldehyde is added to a final concentration of 0.1%. The mixtures are made such that a final total protein concentration of 1 mg/mL is achieved. The mixture is incubated at room temperature for a minimum of about 60 minutes. Free glutaraldehyde is removed, e.g., by dialysis of the reaction mixture against 0.9% sodium chloride, USP. Following dialysis, the Id-KLH conjugate is transferred to a sterile disposable tube.

Packaging/Labeling Process

Purified protein-carrier conjugates such as an Id-KLH conjugate are packaged by aseptically transferring 1 mL of the conjugate in a certified Class 100 BSC into a 2 mL sterile, pyrogen-free polypropylene vial (e.g., such as those from Nalge Nunc International, Rochester, N.Y.). The vials are labeled, and stored at ≦20° C.

Confirmation of Conjugation

In certain embodiments, the product is evaluated by SDS-Polyacrylimide Gel Electrophoresis and by endotoxin testing. The SDS-PAGE is run under reducing conditions to demonstrate the conjugation reaction has run to completion. In the event the conjugation reaction does not run to completion, the heavy and light chain protein species would appear in the SDS-PAGE gel. The absence of these bands confirms completion of the conjugation reaction.

Administration of Drug Product

An immunization cycle may be conducted as follows. The purified tumor-derived antigen protein conjugated to the carrier (e.g., Id-KLH conjugate) is injected subcutaneously at two bilateral sites. Following injection of the protein conjugate on day 1, GM-CSF (Leukine®, Sargramostim; Berlex/Schering AG Germany) is injected subcutaneously at the original injection sites at a dose of 250 μg. GM-CSF alone is injected subcutaneously at the original sites of injection on days 2-4; the GM-CSF dose is divided equally between the two Id-KLH injection sites (i.e., the original injection sites). Multiple immunizations constitute an immunization series.

III. Generating Monoclonal Antibodies

The present invention is not limited by the methods used to generate the monoclonal antibodies or antibody fragments. Monoclonal antibodies may be made in a number of ways, including, for example, using the hybridoma method (e.g. as described by Kohler et al., Nature, 256: 495, 1975, herein incorporated by reference), or by recombinant DNA methods (e.g., U.S. Pat. No. 4,816,567, herein incorporated by reference).

Generally, in the hybridoma method, a mouse or other appropriate host animal, such as a hamster or macaque monkey, is immunized (e.g. with one of the immunogens described in Example 4 below) to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (e.g., Kozbor, J. Immunol., 133: 3001 (1984), herein incorporated by reference).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). After hybridoma cells are identified that produce antibodies of the desired specificity, affinity, and/or activity, the clones may be subcloned by limiting dilution procedures and grown by standard methods. Suitable culture media for this purpose include, for example, D-MEM or RPMI-1640 medium plus fetal bovine serum. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies is described in more detail below.

In some embodiments, antibodies or antibody fragments are isolated from antibody phage libraries generated using the techniques described in, for example, McCafferty et al., Nature, 348: 552554 (1990). Clackson et al., Nature, 352:624-628 (1991) and Marks et al., J. Mol. Biol., 222: 581-597 (1991) describe the isolation of murine and human antibodies, respectively, using phage libraries. Subsequent publications describe the production of high affinity (nM range) human antibodies by chain shuffling (Marks et. al., BioTechnology, 10: 779-783 (1992)), as well as combinatorial infection and in vivo recombination as a strategy for constructing very large phage libraries (e.g., Waterhouse et al., Nuc. Acids. Res., 21: 2265-2266 (1993)). Thus, these techniques, and similar techniques, are viable alternatives to traditional monoclonal antibody hybridoma techniques for isolation of monoclonal antibodies.

The antibodies or antibody fragments can also be prepared, for example, by recombinant expression of immunoglobulin light and heavy chain genes in a host cell. For example, to express a recombinant antibody, a host cell may be transfected with one or more recombinant expression vectors carrying DNA fragments encoding the immunoglobulin light and heavy chains of the antibody such that the light and heavy chains are expressed in the host cell and, preferably, secreted into the medium in which the host cell is cultured, from which medium the antibody can be recovered. Standard recombinant DNA methodologies may be used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al., all of which are herein incorporated by reference.

In certain embodiments, antibodies or antibody fragments are expressed that contain one or more of the CDRs of the present invention (see, e.g., FIGS. 6-11). Such expression can be accomplished by first obtaining DNA fragments encoding the light and heavy chain variable regions. These DNAs can be obtained by amplification and modification of germline light and heavy chain variable sequences using the polymerase chain reaction (PCR). Germline DNA sequences for human heavy and light chain variable region genes are known in the art.

Once the germline VH and VL fragments are obtained, these sequences can be mutated to encode one or more of the CDR amino acid sequences disclosed herein (see, e.g., FIGS. 6-11). The amino acid sequences encoded by the germline VH and VL DNA sequences may be compared to the CDRs sequence(s) desired to identify amino acid residues that differ from the germline sequences. Then the appropriate nucleotides of the germline DNA sequences are mutated such that the mutated germline sequence encodes the selected CDRs (e.g., the six CDRs that are selected from FIGS. 6-11 or variants thereof), using the genetic code to determine which nucleotide changes should be made. Mutagenesis of the germline sequences may be carried out by standard methods, such as PCR-mediated mutagenesis (in which the mutated nucleotides are incorporated into the PCR primers such that the PCR product contains the mutations) or site-directed mutagenesis. In other embodiments, the variable region is synthesized de novo (e.g., using a nucleic acid synthesizer).

Once DNA fragments encoding the desired VH and VL segments are obtained (e.g., by amplification and mutagenesis of germline VH and VL genes, or chemical synthesis, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operably linked to another DNA fragment encoding another polypeptide, such as an antibody constant region or a flexible linker. The isolated DNA encoding the VH region can be converted to a full-length heavy chain gene by operably linking the VH-encoding DNA to another DNA molecule encoding heavy chain constant regions (eg. CH1, CH2 and CH3). The sequences of human heavy chain constant region genes are known in the art (see e.g., Kabat, E. A., et al., (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be, for example, an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operably linked to another DNA molecule encoding only the heavy chain CH1 constant region.

The isolated DNA encoding the VL region can be converted to a full-length light chain gene (as well as a Fab light chain gene) by operably linking the VL-encoding DNA to another DNA molecule encoding the light chain constant region, CL. The sequences of human light chain constant region genes are known in the art (see e.g., Kabat, E. A., et al., (1991) Sequences of Proteins of immunological Interest, Fifth Edition, U.S. Department of Health and Human Services. NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments may be operably linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (see e.g., Huston et al., (1988) Proc. Natl. Acad. Sci. USA 85:5879-5883; and McCafferty et al., (1990) Nature 348:552-554), all of which are herein incorporated by reference).

To express the antibodies, or antibody fragments of the invention, DNAs encoding partial or full-length light and heavy chains, (e.g. obtained as described above), may be inserted into expression vectors such that the genes are operably linked to transcriptional and translational control sequences. In this context, the term “operably linked” is intended to mean that an antibody gene is ligated into a vector such that transcriptional and translational control sequences within the vector serve their intended function of regulating the transcription and translation of the antibody gene. The expression vector and expression control sequences are generally chosen to be compatible with the expression host cell used. The antibody light chain gene and the antibody heavy chain gene can be inserted into separate vectors or, more typically, both genes are inserted into the same expression vector. The antibody genes may be inserted into the expression vector by standard methods (e.g., ligation of complementary restriction sites on the antibody gene fragment and vector, or blunt end ligation if no restriction sites are present). Prior to insertion of the light or heavy chain sequences, the expression vector may already carry antibody constant region sequences. For example, one approach to converting the VH and VL sequences to full-length antibody genes is to insert them into expression vectors already encoding heavy chain constant and light chain constant regions, respectively, such that the VH segment is operably linked to the CH segment(s) within the vector and the VL segment is operably linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention may carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990), herein incorporated by reference. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma virus. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al., all of which are herein incorporated by reference.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634.665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (e.g., for use in dhfr−host cells, or weakly dhfr+host cells, with methotrexate selection/amplification) and the neomycin gene (for G418 selection).

For expression of the light and heavy chains, the expression vector(s) encoding the heavy and light chains may be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

In certain embodiments, the expression vector used to express the antibody and antibody fragments of the present invention are viral vectors, such as retro-viral vectors. Such viral vectors may be employed to generate stably transduced cell lines (e.g. for a continues source of monoclonal antibodies). In some embodiments, the GPEX gene product expression technology (from Gala Design, Inc., Middleton, Wis.) is employed to generate monoclonal antibodies. In particular embodiments, the expression technology described in WO0202783 and WO0202738 to Bleck et al. (both of which are herein incorporated by reference in their entireties) is employed.

In one preferred system for recombinant expression of an antibody, or fragment thereof, a recombinant expression vector encoding both the antibody heavy chain and the antibody light chain is introduced into dhfr−CHO cells by calcium phosphate-mediated transfection. Within the recombinant expression vector, the antibody heavy and light chain genes are each operably linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector may also carry a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In certain embodiments, the antibodies and antibody fragments of the present invention are produced in transgenic animals. For example, transgenic sheep and cows may be engineered to produce the antibodies or antibody fragments in their milk (see, e.g., Pollock DP, et al., (1999) Transgenic milk as a method for the production of recombinant antibodies. J. Immunol. Methods 231:147-157, herein incorporated by reference). The antibodies and antibody fragments of the present invention may also be produced in plants (see, e.g., Larrick et al., (2001) Production of secretory IgA antibodies in plants. Biomol. Eng. 18:87-94, herein incorporated by reference). Additional methodologies and purification protocols are provided in Humphreys et al., (2001) Therapeutic antibody production technologies: molecules applications, expression and purification, Curr. Opin. Drug Discov. Devel. 4:172-185, herein incorporated by reference. In certain embodiments, the antibodies or antibody fragments of the present invention are produced by transgenic chickens (see, e.g., US Pat. Pub. Nos. 20020108132 and 20020028488, both of which are herein incorporated by reference).

IV. Exemplary CDRs For Antibody Humanization

The present invention provides numerous exemplary CDRs, such as those provided in FIGS. 6-11 and the variants discussed below. These can be used to create humanized antibodies; for example, these CDRs can be “grafted” on to human frameworks. In certain embodiments, monoclonal antibodies or antibody fragments are generated with at least one of the CDRs shown in FIGS. 6-11 (or a variant of at least one of these CDRs) using, for example, the recombinant techniques discussed above and/or using the chimeric/humanization techniques discussed below. Preferably, antibodies or antibody fragments composed of at least one of these CDRs are reactive with a framework epitope of an immunoglobulin associated with a human Non-Hodgkin's Lymphoma sample.

The present invention also contemplates sequences that are substantially the same (but not exactly the same) as the CDR sequences (both amino acid and nucleic acid) shown in FIGS. 6-11. For example, one or two amino acid may be changed in the sequences shown in these figures. Also for example, a number of nucleotide bases may be changed in the sequences shown in these figures. Changes to the amino acid sequence may be generated by changing the nucleic acid sequence encoding the amino acid sequence. A nucleic acid sequence encoding a variant of a given CDR may be prepared by methods known in the art using the guidance of the present specification for particular sequences. These methods include, but are not limited to, preparation by site-directed (or oligonucleotide-mediated) mutagenesis, PCR mutagenesis, and cassette mutagenesis of an earlier prepared nucleic acid encoding the CDR. Site-directed mutagenesis is a preferred method for preparing substitution variants. This technique is well known in the art (see, e.g., Carter et al., (1985) Nucleic Acids Res. 13:4431-4443 and Kunkel et. al., (1987) Proc. Natl. Acad. Sci. USA 82:488-492, both of which are hereby incorporated by reference).

Amino acid changes in the CDRs shown in FIGS. 6-11, can be made randomly, based on directed evolution methods (discussed further below), or based on making conservative amino acid substitutions. Conservative modifications in the amino acid sequences of the CDRs may be made based on the various classes of common side-chain properties:
(1) hydrophobic:norleucine, met, ala, val, leu, ile;
(2) neutral hydrophilic:cys, ser, thr;
(3) acidic:asp, glu;
(4) basic:asn, gln, his, lys, arg;
(5) residues that influence chain orientation:gly, pro; and
(6) aromatic:trp, tyr, phe.
   Conservative substitutions will entail exchanging a member of one of these classes for another member of the same class. The present invention also provides the complement of the nucleic acid sequences shown in FIGS. 6-11, as well as nucleic acid sequences that will hybridize to these nucleic acid sequences under low, medium, and high stringency conditions.
   The CDRs of the present invention may be employed with any type of framework. Preferably, the CDRs are used with fully human frameworks, or framework sub-regions. In particularly preferred embodiments, the frameworks are human germline sequences. Examples of fully human frameworks are provided by the NCBI web site which contains the sequences for the currently known human framework regions. Examples of human VH sequences include, but are not limited to, IGHV1-2, IGHV1-3, IGHV1-8, IGHV1-18, IGHV1-24, IGHV1-45, IGHV1-46, IGHV1-58, IGHV1-69, IGHV1-c, IGHV1-f, IGHV2-5, IGHV2-26, IGHV2-70, IGHV3-7, IGHV3-48, IGHV3-9, IGH3-11, IGHV3-13, IGHV3-15, IGHV3-16, IGHV3-19, IGHV3-20, IGHV3-21, IGHV3-23, IGHV3-30, IGHV3-30-3, IGHV3-33, IGHV3-35, IGHV3-38, IGHV3-43, IGHV3-47, IGHV3-49, IGHV3-53, IGHV3-66, IGHV3-72, IGHV3-73, IGHV3-74, IGHV4-4, IGHV4-59, IGHV4-28, IGHV4-30-2, IGHV4-30-4, IGHV4-31, IGHV4-34, IGHV4-39, IGHV4-55, IGHV4-59, IGHV4-61, IGHV4-b, IGHV5-51, IGHV5-a, IGHV6-1, IGHV7-4-1, and IGHV7-81, also see Matsuda et al., (1998) J. Exp. Med. 188:1973-1975, that includes the complete nucleotide sequence of the human immunoglobulin chain variable region locus, herein incorporated by reference. Examples of human VK sequences include, but are not limited to, IGKV1-5, IGKV1-6, IGKV1-8, IGKV1D-8, IGKV1-9, IGKV1-12, IGKV1D-12, IGKV1-12/IGKV1D-12(1), IGKV1-13, IGKV1D-13, IGKV1-16, IGKV1D-16, IGKV1-17, IGKV1D-17, IGKV1-27, IGKV1D-27, IGKV1-33, IGKV1D-33, IGKV1-37, IGKV1D-37, IGKV1-39, IGKV1D-39, IGKV1D-42, IGKV1D-43, IGKV2-24, IGKV2D-24, IGKV2-28, IGKV2D-28, IGKV2-29, IGKV2D-29, IGKV2-30, IGKV2D-30, IGKV2-40, IGKV2D-40, IGKV3-7, IGKV3-11, IGKV3D-11, IGKV3-15, IGKV3D-15, IGKV3-20, IGKV3D-20, IGKV4-1, IGKV5-2, IGKV6-21, IGKV6D-21, and IGKV6D-41, and see Kawasaki et al., (2001) Eur. J. Immunol. 31:1017-1028; Schable and Zachau, (1993) Biol. Chem. Hoppe Seyler 374:1001-1022; and Brensing-Kuppers et al., (1997) Gene 191:173-181, all of which are herein incorporated by reference. Examples of human VL sequences include, but are not limited to, IGLV1-36, IGLV1-40, IGLV1-41, IGLV1-44, IGLV1-47, IGLV1-50, IGLV1-51, IGLV2-8, IGLV2-11, IGLV2-14, IGLV2-18, IGLV2-23, IGLV2-33, IGLV3-1, IGLV3-9, IGLV3-10, IGLV3-12, IGLV3-16, IGLV3-19, IGLV3-21, IGLV 3-22, IGLV3-25, IGLV3-27, IGLV3-32, IGLV4-3, IGLV4-69, IGLV5-39, IGLV5-52, IGLV6-57, IGLV7-43, IGLV7-46, IGLV8-61, IGVL9-49, and IGLV10-54, and see Kawasaki et al., (1997) Genome Res. 7:250-261, herein incorporated by reference. Fully human frameworks can be selected from any of these functional germline genes. Generally, these frameworks differ from each other by a limited number of amino acid changes. These frameworks may be used with the CDRs described herein. Additional examples of human frameworks which may be used with the CDRs of the present invention include, but are not limited to, KOL, NEWM, REI, EU, TUR, TEI, LAY and POM (See, e.g., Kabat et al., (1991) Sequences of Proteins of Immunological Interest, US Department of Health and Human Services, NIH, USA; and Wu et al., (1970), J. Exp. Med. 132:211-250, both of which are herein incorporated by reference).
   V. Chimeric, Humanized, and Human Framework Reactive mAbs

The monoclonal antibodies and antibody fragments of the present invention may be “humanized.” Chimeric antibodies may be produced such that part of the antibody is from one species and part is from a different species. For example, the variable region maybe murine (see, e.g. variable regions in FIGS. 6-11), while the constant regions may be human. Techniques developed for the production of chimeric antibodies, include, for example, Morrison, et al., 1984, Proc. Natl. Acad. Sci., 81, 6851-6855; Neuberger, et al., 1984, Nature 312, 604-608; Takeda, et al., 1985, Nature 314, 452-454, Cabilly et al., U.S. Pat. No. 4,816,567; and Boss et al., U.S. Pat. No. 5,816,397; all of which are herein incorporated by reference. Such techniques generally include splicing the genes from a mouse antibody molecule of appropriate antigen specificity together with genes from a human antibody molecule of appropriate biological activity can be used. In a specific embodiment, the chimeric antibody comprises a variable domain of monoclonal antibody as depicted in FIGS. 6-11, and a human constant region.

The present invention provides humanized and human antibodies. In preferred embodiments, a humanized antibody comprises human antibody amino acid sequences together with amino acid residues that are not from a human antibody. In some embodiments, the human sequences in a humanized antibody comprise the framework regions (FRs) and the sequences or residues that are not from a human antibody comprise one or more complementarity-determining regions (CDRs), such as those shown in FIGS. 6-11.

The residues in a humanized antibody that are not from a human antibody may be residues or sequences imported from or derived from another species (including but not limited to mouse, such as the CDR sequences shown in FIGS. 6-11), or these sequences may be random amino acid sequences (e.g. generated from randomized nucleic acid sequences), which are inserted into the humanized antibody sequence. As noted above, the human amino acid sequences in a humanized antibody are preferably the framework regions, while the residues which are not from a human antibody (whether derived from another species or random amino acid sequences) preferably correspond to the CDRs. However, in some embodiments, one or more framework regions may contain one or more non-human amino acid residues. In cases of alterations or modifications (e.g. by introduction of a non-human residue) to an otherwise human framework, it is possible for the altered or modified framework region to be adjacent to a modified CDR from another species or a random CDR sequence, while in other embodiments, an altered framework region is not adjacent to an altered CDR sequence from another species or a random CDR sequence. In preferred embodiments, the framework sequences of a humanized antibody are entirely human (i.e. no framework changes are made to the human framework).

Non-human amino acid residues from another species, or a random sequence, are often referred to as “import” residues, which are typically taken from an “import” variable domain. Humanization can be essentially performed following the method of Winter and co-workers (e.g., Jones et al., Nature, 321:522-525 (1986); Riechmann et al., Nature, 332:323-327 (1988); Verhoeyen et al., Science, 239:1534-1536 (1988), all of which are hereby incorporated by reference), by substituting rodent (or other mammal) CDRs or CDR sequences for the corresponding sequences of a human antibody. Also, antibodies wherein substantially less than an intact human variable domain has been substituted by the corresponding sequence from a non-human species may also be generated (e.g. U.S. Pat No. 4,816,567, hereby incorporated by reference). In practice, humanized antibodies are typically human antibodies in which some CDR residues and possibly some FR residues are substituted by residues from analogous sites in rodent antibodies, or, as noted above, in which CDR sequences have been substituted by random sequences. By way of non-limiting example only, methods for conferring donor CDR binding affinity onto an antibody acceptor variable region framework are described in WO 01/27160 A1, herein incorporated by reference and in U.S. Pat. No. 6,849,425, both of which are herein incorporated by reference.

The choice of human variable domains, both light and heavy, to be used in making the humanized antibodies is important to reduce antigenicity. According to the so-called “best-fit” method, the sequence of the variable domain of a rodent antibody to be humanized is screened against the entire library of known human variable-domain sequences. The human sequence which is closest to that of the rodent is then accepted as the human framework (FR) for the humanized antibody (e.g., Sims et al., J. Immunol., 151:2296 (1993), and Chothia et al., J. Mol. Biol., 196:901 (1987), both of which are hereby incorporated by reference). Another method uses a particular framework derived from the consensus sequence of all human antibodies of a particular subgroup of light or heavy chains. The same framework may be used for several different humanized antibodies (e.g., Carter et al., Proc. Natl. Acad. Sci. USA, 89:4285 (1992); Presta et al., J. Immunol., 151:2623 (1993), both of which are hereby incorporated by reference).

In other embodiments, there is no need to “pre-select” a particular human antibody framework (i.e. there is no need to select a human framework with the closest homology or sequence identity to a given candidate antibody to be humanized). In these embodiments, a common or universal human framework may be used to accept one or more non-human CDRs. In the preferred embodiment, a single universal, fully human framework is used as the framework for all antibodies to be humanized, regardless of its homology to the framework sequence(s) of the candidate antibodies. In this regard, humanized antibodies may be generated without making any changes in the framework region. This universal, fully human framework can then accept one or more CDR sequences. In one embodiment, the one or more CDR sequences are CDR sequences from an antibody from another species (e.g. mouse or rat) which have been modified in comparison to the corresponding CDR in the intact antibody from the other species (i.e. there is simultaneous introduction of the CDR and modification of the CDR being introduced into the universal human framework). The modification corresponds to one or more amino acid changes (in the modified CDR) in comparison to the corresponding CDR in the intact antibody from the other species. In one embodiment, all amino acid residues in the CDR are included in a library, while in other embodiments, not all of the CDR amino acid residues are included in a library. In another embodiment, the one or more CDR sequences are random sequences, which substitute for CDR sequences.

In preferred embodiments, antibodies are humanized with retention of high affinity for the antigen and other favorable biological properties. In some embodiments, the affinity of the humanized antibody for the antigen is higher than the affinity of the corresponding non-humanized, intact antibody or fragment or portion thereof (e.g. the candidate rodent antibody). In this regard, in some embodiments, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen (s), is achieved. In general, the CDR residues are directly and most substantially involved in influencing antigen binding.

A variety of specific methods, well known to one of skill in the art, may be employed to introduce antibody CDRs (or random sequences substituting for antibody CDRs) into antibody frameworks. In some embodiments, overlapping oligos may be used to synthesize an antibody gene, or portion thereof (for example, a gene encoding a humanized antibody). In other embodiments, mutagenesis of an antibody template may be carried out using the methods of Kunkel (Proc. Natl. Acad. Sci. USA 82:488-492 (1985)), for example to introduce a modified CDR or a random sequence to substitute for a CDR. In some embodiments, light and heavy chain variable regions are humanized separately, and then co-expressed as a humanized variable region. In other embodiments, humanized variable regions make-up the variable region of an intact antibody. In some embodiments, the Fc region of the intact antibody comprising a humanized variable region has been modified (e.g. at least one amino acid modification has been made in the Fc region). For example, an antibody that has been humanized with randomized CDR and no framework changes may comprise at least one amino acid modification in the Fc region.

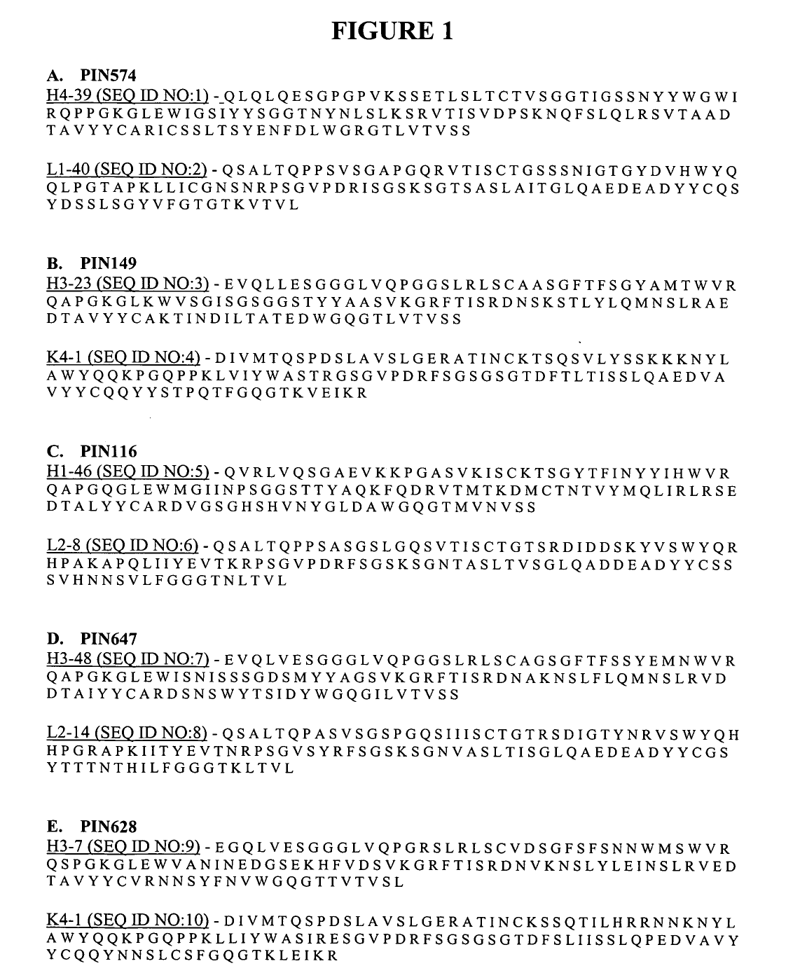

In other embodiments, transgenic animals (e.g., mice) that are capable, upon immunization, of producing a full repertoire of human antibodies in the absence of endogenous immunoglobulin production are employed (e.g. immunized with sequences shown in FIG. 1). Therefore, in certain embodiments, the antibodies and antibody fragments of the present invention are fully human. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge (See, e.g., U.S. Pat. No. 6,162,963, Pat. Pub. US2003/0070185, Jakobovits et al., Proc. Natl. Acad. Sci. USA, 90: 2551 (1993), and Jakobovits et al., Nature, 362:255-258 (1993), all of which are hereby incorporated by reference in their entirities). Human antibodies can also be derived from phage-display libraries (e.g., Hoogenboom et al., J. Mol. Biol., 227:381 (1991), and Vaughan et al. Nature Biotech 14:309 (1996), both of which are hereby incorporated by reference).

The grafted CDRs for humanization methods, as mentioned above, may be subjected to directed evolution type procedures in order to retain or increase the binding affinity of the final antibody or antibody or antibody fragment. For example, the CDRs shown in FIGS. 6-11 may be subjected to directed evolution procedures such that alternative frameworks can be employed without a loss of binding affinity. Such techniques are described, for example, in U.S. Pat. Pub. 20040162413, herein incorporated by reference. Generally, such directed evolution type methods effectively combines CDR grafting procedures and affinity reacquisition of the grafted variable region into a single step. The methods of the invention also are applicable for affinity maturation of an antibody variable region. The affinity maturation process can be substituted for, or combined with the affinity reacquisition function when being performed during a CDR grafting procedure. Alternatively, the affinity maturation procedure can be performed independently from CDR grafting procedures to optimize the binding affinity of variable region, or an antibody. An advantage of combining grafting and affinity reacquisition procedures, or affinity maturation, is the avoidance of time consuming, step-wise procedures to generate a grafted variable region, or antibody, which retains sufficient binding affinity for therapeutic utility. Therefore, therapeutic antibodies can be generated rapidly and efficiently using the methods of the invention. Such advantages beneficially increase the availability and choice of useful therapeutics for human diseases as well as decrease the cost to the developer and ultimately to the consumer.

VI. Therapeutic Formulations and Uses

The monoclonal antibodies and antibody fragments of the present invention (e.g., reactive with a framework epitope of an immunoglobulin present on a human's Non-Hodgkin's Lymphoma cells) are useful for treating a subject with a disease. These antibodies may also be used in diagnostic procedures. In preferred embodiments, the antibodies are administered to a patient with B cell lymphoma, which is generally characterized by unabated B cell proliferation.

In some embodiments, the antibodies are conjugated to various radiolabels for both diagnostic and therapeutic purposes. Radiolabels allow “imaging” of tumors and other tissue, as well helping to direct radiation treatment to tumors. Exemplary radiolabels include, but are not limited to, ¹³¹I, ¹²⁵I, ¹²³I, ⁹⁹Tc, ⁶⁷Ga, ¹¹¹In, ¹⁸⁸Re, ¹⁸⁶Re, and preferably, ⁹⁰Y.

In certain embodiments, the disease treated is Non-Hodgkin's lymphoma (NHL). In other embodiments, the disease treated includes any BCR (B cell antigen receptor) expressing B cell malignacies. In some embodiments, the disease is selected from relapsed Hodgkin's disease, resistant Hodgkin's disease high grade, low grade and intermediate grade Non-Hodgkin's lymphomas (NHLs), B cell chronic lymphocytic leukemia (B-CLL), lymphoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic; follicular, diffuse large cell; diffuse small cleaved cell; large cell immunoblastic lymphoblastoma; small, non-cleaved; Burkift's and non-Burkitt's; follicular, predominantly large cell; follicular, predominantly small cleaved cell; follicular, mixed small cleaved and large cell lymphomas, and systemic lupus erythematosus (SLE). In particular embodiments, the disease treated is Waldenstrom's Macroglobulinemia (WM) or Chronic Lymphocytic Leukemia (CLL).

In some embodiments, the antibodies of the present invention are used for treatment of diseases such as Waldenstrom's macroglobulianemia, multiple myeloma, plasma cell dyscrasias, chronic lymphocytic leukemia, treatment of transplant, hairy cell leukemia, ITP, Epstein Barr virus lymphomas after stem cell transplant, and Kidney transplant, see U.S. Pat. Pub. 20020128448, herein incorporated by reference. In other embodiments, the antibodies of the present invention are used for the treatment of a disease selected from the group consisting of B cell lymphomas, leukemias, myelomas, autoimmune disease, transplant, graft-vs-host disease, infectious diseases involving B cells, lymphoproliferation diseases, and treatment of any disease or condition wherein suppression of B cell activity and/or humoral immunity is desirably suppressed. In certain embodiments, the antibodies of the present invention are used for the treatment of a disease selected from the group consisting of B cell lymphomas, leukemia, myeloma, transplant, graft-vs-host disease, autoimmune disease, lymphoproliferation conditions, and other treatment diseases and conditions wherein the inhibition of humoral immunity, B cell function, and/or proliferation, is therapeutically beneficial. In further embodiments, the antibodies of the present invention are used for the treatment of B-ALL, Hairy cell leukemia, Multiple myeloma, Richter Syndrome, Acquired Factor VIII inhibitors, Antiphospholipid syndrome, Autoimmune hemolytic anemia, Autoimmune thrombocytopenia, Bullous pemphigoid, Cold hemagglutinin disease, Evan's Syndrome, Goodpasture's syndrome, Idiopathic membranous nephropathy, Idiopathic thrombocytopenic purpura, IgM associated polyneuropathy, Kaposi sarcoma-associated herpesvirus (KSHV)-related multicentric Castleman disease (MCD), Myasthenia gravis, Pemphigus vulgaris, Primary biliary cirrhosis, Pure red cell aplasia, Rheumatoid arthritis, Sjogren's Syndrome, Systemic immune complex vasculitis, Systemic lupus erythematosus, Type II mixed cryoglobulinemia, Wegener's granulomatosis, Allograft rejection, Post-transplant lymphoproliferative disease, or Purging of stem cells for bone marrow transplantation.

The antibodies of the present invention may also be administered in combination with other therapeutic moieties. For example, the antibodies of the present invention may be administered a part of a chemotherapeutic program (e.g. CHOP), whether before or after. The antibodies of the present invention may also be administered before, after or with cytokines, G-CSF, or IL-2 (See, U.S. Pat. No. 6,455,043, herein incorporated by reference).

The antibodies and antibody fragments of the present invention may be administered by any suitable means, including parenteral, non-parenteral, subcutaneous, topical, intraperitoneal, intrapulmonary, intranasal, and intralesional administration (e.g., for local immunosuppressive treatment). Parenteral infusions include, but are not limited to, intramuscular, intravenous, intra-arterial, intraperitoneal, or subcutaneous administration. In addition, antibodies are suitably administered by pulse infusion, particularly with declining doses. Preferably, the dosing is given by injections, most preferably intravenous or subcutaneous injections, depending in part on whether the administration is brief or chronic.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is advantageous to formulate parenteral compositions in dosage unit form for ease of administration and uniformity of dosage. The dosages of the antibodies of the present invention are generally dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody fragment is 0.1-20 mg/kg, more preferably 1-10 mg/kg. In some embodiments, the dosage is from 50-600 mg/m² (e.g. 375 mg/m²). It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the present invention.

The dosage administered will, of course, vary depending upon known factors such as the pharmacodynamic characteristics of the particular agent, its mode and route of administration, the age, health, and weight of the recipient, the nature and extent of symptoms, the kind of concurrent treatment, the frequency of treatment, and the effect desired. For example, a daily dosage of active ingredient can be about 0.01 to 100 milligrams per kilogram of body weight. Ordinarily 1 to 5, and preferably 1 to 10 milligrams per kilogram per day given in divided doses 1 to 6 times a day or in sustained release form, may be effective to obtain desired results.

The antibody and antibody fragments of the invention can be incorporated into pharmaceutical compositions suitable for administration to a subject. For example, the pharmaceutical composition may comprise an antibody or antibody fragment and a pharmaceutically acceptable carrier. As used herein, “pharmaceutically acceptable carrier” includes solvents, dispersion media, coatings, antibacterial and antifungal agents, isotonic and absorption delaying agents, and the like that are physiologically compatible. Examples of pharmaceutically acceptable carriers include one or more of the following: water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibodies of the present invention.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies.

Therapeutic compositions typically are sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody fragment) in the required amount in an appropriate solvent with one or a combination of ingredients enumerated above, as required, followed by sterile filtration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (see, e.g., Sustained and Controlled Release Drug Delivery Systems, J. R. Robinson. ed., Marcel Dekker, Inc., New York, 1978).

In certain embodiments, the binding molecules of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

The pharmaceutical compositions of the invention may include a “therapeutically effective amount” or a “prophylactically effective amount” of an antibody or antibody fragment of the invention. A “therapeutically effective amount” refers to an amount effective, at dosages and for periods of time necessary, to achieve the desired therapeutic result. A therapeutically effective amount of the antibody or antibody fragment may vary according to factors such as the disease state, age, sex, and weight of the individual, and the ability of the antibody or antibody fragment to elicit a desired response in the individual. A therapeutically effective amount is also one in which any toxic or detrimental effects of the antibody or antibody fragment are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

VII. Vectors

A. Expression Vectors

Any type of expression vector may be used with the present invention. In certain embodiments, the expression vectors comprise a number of genetic elements: A) a plasmid backbone; B) regulatory elements which permit the efficient expression of genes in eukaryotic cells—these include, but are not limited to, enhancer/promoter elements, poly A signals and splice junctions; C) polylinkers which allow for the easy insertion of a gene (i.e., for example, a selectable marker gene, an amplifiable marker gene, or a gene of interest) into the expression vector; and D) constructs showing the possible combination of the genetic elements. These genetic elements may be present on the expression vector in a number of configurations and combinations.

Plasmid Backbone

In some embodiments, the expression vectors contain plasmid sequences which allow for the propagation and selection of the vector in procaryotic cells; these plasmid sequences are referred to as the plasmid backbone of the vector. While not intending to limit the invention to a particular plasmid, the following plasmids are described as examples.

The pUC series of plasmids and their derivatives which contain a bacterial origin of replication (the pMB 1 replicon) and the β-lactamase or ampicillin resistance gene. The pUC plasmids, including, but not limited to, pUC18 (ATCC 37253) and pUC19 (ATCC 37254), are are believed to be expressed at high copy number (500-700) in bacterial hosts. pBR322 and its derivatives which contain the pMB 1 replicon and genes which confer ampicillin and tetracycline resistance. pBR322 may be expressed at 15-20 copies per bacterial cell. pUC and pBR322 plasmids are commercially available from a number of sources (for example, Gibco BRL, Gaithersburg, Md.).

Regulatory Elements

The transcription of each cDNA may be directed by genetic elements which allow for high levels of transcription in the host cell. Each cDNA is under the transcriptional control of a promoter and/or enhancer. Promoters and/or enhancers are short arrays of DNA which direct the transcription of a linked gene. While not intending to limit the invention to the use of any particular promoter and/or enhancer elements, the following promoter and/or enhancer elements exemplify some embodiments contemplated by the present invention because they are believed to direct high levels of expression of operably linked genes in a wide variety of cell types. For example, the SV40 and SR-α enhancer and/or promoters may be used when the vector is to be transfected into a host cell which expresses the SV40 T antigen as these enhancer and/or promoter sequences contain the SV40 origin of replication.

The SV40 enhancer/promoter is very active in a wide variety of cell types from many mammalian species. Dijkema et al., “Cloning and expression of the chromosomal immune interferon gene of the rat” EMBO J., 4:761 (1985). The SR-α enhancer/promoter comprises the R-U5 sequences from the LTR of the human T-cell leukemia virus-1 (HTLV-1) and sequences from the SV40 enhancer/promoter. Takebe et al., “SR alpha promoter: an efficient and versatile mammalian cDNA expression system composed of the simian virus 40 early promoter and the R-U5 segment of human T-cell leukemia virus type 1 long terminal repeat” Mol. Cell. Biol., 8:466 (1988). The HTLV-1 sequences may be placed immediately downstream of the SV40 early promoter. These HTLV- 1 sequences are located downstream of the transcriptional start site and are present as 5′ nontranslated regions on the RNA transcript. The addition of the HTLV-1 sequences increases expression from the SV40 enhancer/promoter. The human cytomegalovirus (CMV) major immediate early gene (IE) enhancer/promoter has been reported to be active in a broad range of cell types. Boshart et al., “A very strong enhancer is located upstream of an immediate early gene of human cytomegalovirus” Cell 41:521 (1985). The 293 cell line (ATCC CRL 1573), an adenovirus transformed human embryonic kidney cell line, is particularly advantageous as a host cell line for vectors containing the CMV enhancer/promoter as the adenovirus IE gene products increase the level of transcription from the CMV enhancer/promoter. Graham et al., “Characteristics of a human cell line transformed by DNA from human adenovirus type 5 ”J Gen. Virol., 36:59 (1977); Harrison et al., “Host-range mutants of adenovirus type 5 defective for growth in HeLa cells” Virology 77:319 (1977); and Graham et al., “Defective transforming capacity of adenovirus type 5 host-range mutants” Virology 86:10 (1978). The enhancer/promoter from the LTR of the Moloney leukemia virus is a strong promoter and has been reported to be active in a broad range of cell types. Laimins et al., “Host-specific activation of transcription by tandem repeats from simian virus 40 and Moloney murine sarcoma virus” Proc. Natl. Acad. Sci. USA 79:6453 (1984). The enhancer/promoter from the human elongation factor 1α gene and has been reported as abundantly transcribed in a very broad range of cell types. Uetsuki et al., “Isolation and characterization of the human chromosomal gene for polypeptide chain elongation factor-1 alpha” J. Biol. Chem., 264:5791 (1989); and Mizushima et al., “pEF-BOS, a powerful mammalian expression vector” Nucl. Acids. Res. 18:5322 (1990).

In certain embodiments, a cDNA coding region is followed by a polyadenylation (poly A) element. In certain embodiments, poly A elements of the present invention are strong signals that result in efficient termination of transcription and polyadenylation of the RNA transcript. For example, a heterologous poly A element may be a SV40 poly A signal (See SEQ ID NO:3). Alternatively, a heterologous poly A element may be a poly A signal from the human elongation factor 1α (hEF1α) gene. (See SEQ ID NO:41). The invention is not limited by the poly A element utilized. The inserted cDNA may utilize its own endogenous poly A element provided that the endogenous element is capable of efficient termination and polyadenylation.

In certain embodiments, the present invention provides an expression vectors comprising a splice junction sequence. Although it is not necessary to understand the mechanism of an invention, it is believed that splicing signals mediate the removal of introns from the primary RNA transcript and consist of a splice donor and acceptor site. It is further believed that the presence of splicing signals on an expression vector often results in higher levels of expression of the recombinant transcript. In certain embodiments, a splice junction comprises a splice junction from the 16S RNA of SV40. In another embodiment, a splice junction is the splice junction from the hEF1α gene. The invention is not limited by the use of a particular splice junction. The splice donor and acceptor site from any intron-containing gene may be utilized.

In certain embodiments, the present invention provides an expression vector comprising a polylinker which allows for the easy insertion of DNA segments into the vector. In certain embodiments, a polylinker comprises a short synthetic DNA fragment which contains the recognition site for numerous restriction endonucleases. Any desired set of restriction sites may be utilized in a polylinker. In some embodiments, a polylinker sequence may comprise an SD5 or SD7 polylinker sequences. For example, an SD5 polylinker may be formed by the SD5A (SEQ ID NO:1) and SD5B (SEQ ID NO:2) oligonucleotides and contains the recognition sites for XbaI, NotI, SfiI, SacII and EcoRI. Alternatively, an SD7 polylinker may be formed by the SD7A (SEQ ID NO:4) and SD7B (SEQ ID NO:5) oligonucleotides and contains the following restriction sites: XbaI, EcoRI, MluI, StuI, SacII, SfiI, NotI, BssHII and SphI. In some embodiments, A polylinker sequence may be located downstream of the enhancer/promoter and splice junction sequences and upstream of the poly A sequence. Although it is not necessary to understand the mechanism of an invention, it is believed that insertion of a cDNA or other coding region (i.e., a gene of interest) into the polylinker allows for the transcription of the inserted coding region from the enhancer/promoter and the polyadenylation of the resulting RNA transcript.

The above elements may be arranged in numerous combinations and configurations to create the expression vectors of the invention. The genetic elements are manipulated using standard techniques of molecular biology known to those skilled in the art. Sambrook et al., In: Molecular Cloning:A Laboratory Manual, 2nd ed., Cold Spring Harbor Laboratory Press, New York (1989). Once a suitable recombinant DNA vector has been constructed, the vector can be introduced into any desired host cell. DNA molecules are known to be transfected into prokaryotic hosts using standard protocols. Briefly, host cells may be made competent by treatment with, for example, calcium chloride solutions. Alternatively, competent bacteria cells are commercially available and/or are easily made in the laboratory. The induction of host cell competence permits the uptake of DNA by the bacterial cell. Another example for introducing DNA into bacterial cells is electroporation in which an electrical pulse is used to facilitate the uptake of DNA by bacterial cells.

Following the introduction of DNA into a host cell, selective pressure may be applied to isolate those cells which have taken up the DNA. Prokaryotic vectors (i.e., for example, plasmids) may contain an antibiotic-resistance gene, such as, but not limited to, ampicillin, kanamycin, or tetracycline resistance genes. In certain embodiments, a pUC plasmid comprises an ampicillin resistance gene. Although it is not necessary to understand the mechanism of an invention, it is believed that growth in the presence of an appropriate antibiotic indicates the presence of the vector DNA.

For analysis to confirm correct sequences in plasmids constructed, a ligation mixture may be used to transform suitable strains of E. coli. Examples of commonly used E.coli strains include, but are not limited to, the HB101 strain (Gibco BRL), TG1 and TG2 (derivatives of the JMO101 strain), DH10B strain (Gibco BRL) or K12 strain 294 (ATCC No. 31446). It is known that plasmids from transformants may be prepared, analyzed by digestion with restriction endonucleases, and/or sequenced. Messing et al., “A system for shotgun DNA sequencing” Nucl. Acids Res., 9:309 (1981).

Plasmid DNA may be purified from bacterial lysates by chromatography on Qiagen Plasmid Kit columns (Qiagen, Chatsworth, Calif.) according to the manufacturer's directions for large scale preparation.

Small scale preparation (i.e., for example, minipreps) of plasmid DNA may be performed by alkaline lysis. Bimboim et al., “A rapid alkaline extraction procedure for screening recombinant plasmid DNA” Nucl. Acids. Res., 7:1513 (1979). Briefly, bacteria harboring a plasmid is grown in the presence of the appropriate antibiotic (i.e., for example, 60 μg/ml ampicillin for pUC-based plasmids) overnight at 37° C. with shaking. 1.5 ml of the overnight culture may then be transferred to a 1.5 ml microcentrifuge tube. The bacteria may be pelleted by centrifugation at 12,000 g for 30 seconds in a microcentrifuge. The supernatant may be removed by aspiration. The bacterial pellet may be resuspended in 100 μl of ice-cold Solution I comprising 50 mM glucose, 25 mM Tris-HCl, pH 8.0 and 10 mM EDTA at a pH 8.0. Two hundred μl of Solution II comprising 0.2 N NaOH and 1% SDS may then be added and the tube is inverted to mix the contents. 150 μl of ice-cold Solution III comprising 3 M sodium acetate adjusted to pH 4.8 with glacial acetic acid may be added and the tube is vortexed to mix the contents. The tube is then placed on ice for 3 to 5 minutes. The tube is then centrifuged at 12,000 g for 5 minutes in a microcentrifuge and the supernatant is transferred to a fresh tube. The plasmid DNA is precipitated using 2 volumes of ethanol at room temperature and incubating 2 minutes at room temperature (approximately 25° C.). The DNA is pelleted by centrifugation at 12,000 g for 5 minutes in a microcentrifuge. The supernatant is removed by aspiration and the DNA pellet is resuspended in a suitable buffer such as TE buffer (10 mM Tris-HCl, pH 7.6, 1 mM EDTA, pH 8.0).

Expression vector DNA purified by either chromatography on Qiagen columns or by the alkaline lysis miniprep method is suitable for use in transfection experiments.

B. Amplification Vectors

A vector encoding a structural gene which permits the selection of cells containing multiple or “amplified” copies of the vector encoding the structural gene may be referred to as an amplification vector. An amplifiable gene is believed to respond either to an inhibitor or lack of an essential metabolite by amplification to increase the expression product (i.e., for example, a protein encoded by the amplifiable gene). An amplifiable gene may also be characterized as being able to complement an auxotrophic host. For example, the gene encoding dihydrofolate reductase (DHFR) may be used as the amplifiable marker in conjunction with cells lacking the ability to express a functional DHFR enzyme. However, it is not necessary to use an auxotrophic host cell. In certain embodiments, the present invention provides a host cell that is not auxotrophic with respect to the amplifiable marker.

The present invention is not limited by the use of a particular amplifiable gene. Various expressible genes may be employed including, but not limited to, DHFR, carbamoyl phosphate synthetase-aspartate carbamoyltransferase-dihydroorotase (CAD), metallothioneins, asparagine synthetase, glutamine synthetase, or surface membrane proteins exhibiting drug resistance. By blocking a metabolic process in the cells with enzyme inhibitors, such as methotrexate, for DHFR or cytotoxic agents such as metals, with the metallothionein genes, or by maintaining a low or zero concentration of an essential metabolite, the cellular response will be amplification of the particular gene and flanking sequences. Kaufman et al., “Amplification and expression of sequences cotransfected with a modular dihydrofolate reductase complementary dna gene” J Mol. Biol. 159:601 (1982). Because it is known that the process of gene amplification results in the amplification of the amplifiable marker and surrounding DNA sequences, it is possible to co-amplify gene sequences other than those encoding the amplifiable marker. Kaufman et al., “Coamplification and coexpression of human tissue-type plasminogen activator and murine dihydrofolate reductase sequences in Chinese hamster ovary cells” Mol. Cell. Biol. 5:1750 (1985). For example, an amplification of sequences encoding a gene of interest may be accomplished by co-introducing sequences encoding the gene of interest and the amplifiable marker into the same host cell.

A gene encoding a protein of interest may be physically linked to the amplifiable marker by placing both coding regions with appropriate regulatory signals on a single vector. However, it is not necessary that both coding regions be physically located on the same vector. Because small vector molecules are believed to be easier to manipulate and give higher yields when grown in bacterial hosts, one embodiment of the present invention provides a gene of interest and the amplifiable marker gene located on two separate plasmid vectors. Whether an amplifiable marker and a gene of interest are encoded on the same or separate vector plasmids, vector molecules may be linearized by digestion with a restriction enzyme prior to introduce the vector DNAs into a host cell. A useful restriction enzyme utilized is generally selected for its ability to cut within the plasmid backbone of the vector but not cut within the regulatory signals or the coding region of the amplifiable marker or gene of interest.

In certain embodiments, an amplification vector may be constructed by placing a desired structural gene encoding an amplifiable marker into an expression vector such that the regulatory elements present on the expression vector direct the expression of the product of the amplifiable gene. The invention may be illustrated by using a structural gene encoding DHFR as the amplifiable marker. For example, DHFR coding sequences may be placed in a polylinker region of the expression vector pSSD7 such that the DHFR coding region is under the transcriptional control of the SV40 enhancer/promoter. The invention is not limited by the selection of any particular vector for the construction of the amplification vector. Any suitable expression vector may be utilized. In certain embodiments, expression vectors include, but are not limited to, pSSD5, pSSD7, pSR.alpha.SD5, pSR.alpha.SD7, pMSD5, or pMSD7. Although it is not necessary to understand the mechanism of an invention, it is believed that these expression vectors utilize regulatory signals which permit high level expression of inserted genes in a wide variety of cell types. In certain embodiments, the amplification vectors employed are those described in U.S. Pat. Nos. 5,972,334 and 5,776,746, both of which are herein incorporated by reference in their entireties.

C. Selection Vectors

It is generally known in the art that an expression vector encoding a selectable marker gene may be referred to as a selection vector. In certain embodiments, a selectable marker comprises a dominant selectable marker. Examples of dominant selectable markers include, but are not limited to, a neo gene, a hyg gene, or a gpt gene. Alternatively, a selectable marker may utilize a host cell which lacks an ability to express the product encoded by the selectable marker (i.e., for example, a non-dominant marker). Examples of such non-dominant markers include, but are not limited to, a tk gene, a CAD gene, or a hprt gene.

The invention is not limited to the use of a particular selectable marker or to the use of any selectable marker. In certain embodiments, the host cell comprises a hypoxanthine-guanine phosphoribosyl transferase (HPRT)-deficient cell line and an amplifiable marker, wherein the marker comprises DHFR.

When an HPRT-deficient cell line is utilized and this cell line produces a functional DHFR enzyme, a selectable marker encoding the HPRT enzyme may be utilized. Alternatively, a host cell may be co-transfected with plasmids containing a selectable marker (i.e., for example, HPRT), an amplifiable marker (i.e., for example, DHFR), and one or more proteins of interest. Although it is not necessary to understand the mechanism of an invention, it is believed that transfected cells are then first selected for the ability to grow in HxAz medium (hypoxanthine and azaserine) which requires the expression of HPRT by the cell. It is further believed that the cells having the ability to grow in HxAz medium incorporate at least the selection vector encoding HPRT. Because the vector DNAs may then be linearized and introduced into a host cell (i.e., for example, by electroporation), cells which have taken up the HPRT vector are also likely to have taken up the vectors encoding DHFR, and the protein(s) of interest. This is because linearized vectors are known to form long concatemers or tandem arrays which integrate with a very high frequency into the host chromosomal DNA as a single unit. Toneguzzo et al., “Electric field-mediated gene transfer:characterization of DNA transfer and patterns of integration in lymphoid cells” NucL. Acid Res. 16:5515 (1988).

In certain embodiments, the present invention provides selecting a transfected cell expressing HPRT comprising DHFR as the amplifiable marker in a cell line which is not DHFR-deficient. Although it is not necessary to understand the mechanism of an invention, it is believed that the use of the selectable marker allows the circumvention of the problem of amplification of the host cell's endogenous DHFR gene. Walls et al., “Amplification of multicistronic plasmids in the human 293 cell line and secretion of correctly processed recombinant human protein C” Gene 81:139-49 (1989). However, the present invention can be practiced without using a selectable marker in addition to the amplification vector when cell lines which are not DHFR-deficient are employed. For example, when an amplifiable marker comprises a dominant amplifiable marker, including but not limited to, a glutamine synthetase gene or where the host cell line lacks the ability to express the amplifiable marker (i.e., for example, a DHFR- cell line), no selectable marker need be employed.

VIII. Cell Lines and Cell Culture

A variety of mammalian cell lines may be employed for the expression of recombinant proteins according to the methods of the present invention. Exemplary cell lines include, but are not limited to, Chinese Hamster Ovary (CHO) cell lines, for example, CHO-K1 cells (ATCC CCl 61; ATCC CRL 9618) and/or derivations thereof such as, but not limited to, DHFR⁻ CHO-KI cell lines (i.e., for example, CHO/DHFR⁻; ATCC CRL 9096), mouse L cells, and BW5147 cells and variants thereof such as, but not limited to, BW5147.3 (ATCC TIB 47) and BW5147.G.1.4 cells (ATCC TIB 48). The cell line employed may grow attached to a tissue culture vessel (i.e, attachment-dependent) or may grow in suspension (i.e., attachment-independent).

In certain embodiments, the cell culture comprises BW5147.G.1.4 cells. Although it is not necessary to understand the mechanism of an invention, it is believed that BW5147.G.1.4 cells have a very rapid doubling time (i.e., a doubling time of about 12 hours when grown in RPMI 1640 medium containing 10% Fetal Clone I (Hyclone®)). It is further believed that the doubling time or generation time refers to the amount of time required for a cell line to increase the number of cells present in the culture by a factor of two. In contrast, the CHO-K1 cell line (from which the presently available DHFR⁻ CHO-KI cell lines were derived) are believed to have a doubling time of about 21 hours when the cells were grown in either DMEM containing 10% Fetal Clone II (Hyclone®) or Ham's F-12 medium containing 10% Fetal Clone II®.

A rapid doubling time is advantageous because as the more rapidly a cell line doubles, the more rapidly amplified variants of the cell line will appear and produce colonies when grown in medium which requires the expression of the amplifiable marker. Small differences in the doubling times (i.e., 1-2 hours) between cell lines generate large differences in the amount of time required to select for a cell line having useful levels of amplification which result in a high level of expression of the non-selectable gene product. A short isolation time a high expressing cell line can be advantageous. For example, when producing proteins to be used in clinical applications (e.g., the production of tumor-related proteins to be used to immunize a cancer patient).

In certain embodiments, BW5147.G.1.4 cells permit the amplification of a non-selectable gene encoding a protein of interest at a very high frequency. Using the methods of the present invention, about 80% of BW5147.G. 1.4 cells which survive growth in the selective medium (e.g., HxAz medium) will amplify input DNA comprising an amplifiable marker and DNA encoding a protein of interest. In certain embodiments, amplification may be measured by the ability of the cells to survive in medium containing methotrexate (MTX) and the production of increased amounts of the protein of interest. For example, 80% of the cells which survive growth in the selective medium will survive growth in medium while expressing an amplifiable marker. Although it is not necessary to understand the mechanism of an invention, it is believed that when cells are subjected to growth in medium containing a compound(s) which requires expression of the amplifiable marker (e.g., growth in the presence of MTX requires the expression of DHFR), the cells which survive are said to have been subjected to a round of amplification. Following an initial (i.e., first) round of amplification, cells may be placed in a medium containing an increased concentration of the compounds which require expression of the amplifiable marker and the cells which survive growth in this increased concentration are said to have survived a second round of amplification. Another round of selection in medium containing yet a further increase in the concentration of the compounds which require expression of the amplifiable marker is referred to as the third round of amplification.

Of those transfected BW5147.G.1.4 clones which amplify in the first round of amplification (as measured by both the ability to grow in increased concentrations of MTX and an increased production of the protein of interest), about ⅔also coordinately amplify an amplifiable gene as well as the gene encoding the protein of interest in the second round of amplification. All clones which coordinately amplified an amplifiable marker and a gene encoding the protein of interest in the second round of amplification have been found to coordinately amplify both genes in all subsequent rounds of amplification.

An additional advantage of using BW5147.G. 1.4 cells is the fact that these cells are very hardy. A cell line is said to be hardy when it is found to be able to grow well under a variety of culture conditions. Hardiness may further be defined herein as an ability to be revived after being allowed to remain in medium which has exhausted the buffering capacity or which has exhausted certain nutrients. Hardiness also denotes that a cell line is easy to work with and it grows robustly.

BW5147.G.1.4 cells may be maintained by growth in DMEM containing 10% FBS or RPMI 1640 medium containing 10% Fetal Clone I®. CHO-K1 cells (ATCC CCl 61, ATCC CRL 9618) may be maintained in DMEM containing 10% Fetal Clone II (Hyclone®), Ham's F12 medium containing 10% Fetal Clone II® or Ham's F12 medium containing 10% FBS and CHO/dhFr- cells (CRL 9096) may be maintained in Iscove's modified Dulbecco's medium containing 0.1 mM hypoxanthine, 0.01 mM thymidine and 10% FBS. Those having ordinary skill in the art usually grow these cell lines in a humidified atmosphere containing 5% CO₂ at a temperature of 37° C.

The invention is not limited by the choice of a particular host cell line. Any cell line can be employed in the methods of the present invention. In certain embodiments, cell lines have a rapid rate of growth or a low doubling time (i.e., for example, a doubling time of 15 hours or less) and may be capable of amplifying an amplifiable marker at a reasonable rate without amplification of the endogenous locus at a similar or higher rate. Although it is not necessary to understand the mechanism of an invention, it is believed that cell lines which have the ability to amplify the amplifiable marker at a rate which is greater than the rate at which the endogenous locus is amplified are identified by finding that the ability of the cell to grow in increasing concentrations of the inhibitor (i.e., the compound which requires the cell to express the amplifiable marker in order to survive) correlates with an increase in the copy number of the amplifiable marker (this may be measured directly by demonstrating an increase in the copy number of the amplifiable marker by Southern blotting, quantitative PCR, or in situ hybridization techniques or indirectly by demonstrating an increase in the amount of mRNA produced from the amplifiable marker by Northern blotting).

It is known that by using the biochemical properties of the amino acids from the primary structure of proteins epitopes may be predicted (i.e., for example, B-cell epitopes). For example, B-cell epitopes may contain either solvent exposed and hydrophilic residues that are useful in their identification. mAbs generated with peptides can recognize linear epitopes but often with lower affinity binding and/or do not recognize the native sequence. Alternatively, conformationally-dependent epitopes (i.e., non-linear epitopes) are more likely to have higher binding affinities and recognize native protein. Because B-cell epitope prediction involves the identification of multiple epitopes in non-sequential sequences (i.e., for example, framework regions) within a large protein, the process is expected to be less robust than epitope prediction involving sequential sequences. Consequently, an empirical process is best used to evaluate current biological theories thought to influence immune recognition and most likely to result in a successful immunogen selection.

Such an empirical process is demonstrated within the Examples below. It is not intended that the Examples represent any limitations upon the invention but are offered merely as representative embodiments.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply:M (molar); mM (millimolar); nM (nanomolar); pM (picomolar); mg (milligrams); μg (micrograms); pg (picograms); ml (milliliters); μl (microliters); ° C.(degrees Celsius); OD (optical density); nm (nanometer); BSA (bovine serum albumin); and PBS (phosphate-buffered saline solution).

### EXAMPLE 1

### Determining Variable Region Utilization in Tumor Associated Idiotypic Proteins from a Non-Hodgkin's B Cell Lymphoma Patient Population

This example describes a determination of the variable region utilization of tumor-associated idiotypic proteins from a Non-Hodgkin's B Cell lymphoma patient population composed of over 500 patients. The first domain of the V region of an idiotypic protein is called framework 1 (FRI), which is about 25 amino acids in length and can be used to group the V region genes into families. There is more homology (>80% in FR1) within a family than between any two different families. The role of the FR is to create a scaffold for the CDRs to form the antigen-binding site. To ensure productive Ig folding amino acid usage in FR is more constrained than that for the CDRs.

To classify each of the Non-Hodgkin's B Cell lymphoma patients, the following was performed for each patient sample. First, suitable tumor samples are obtained at the clinical sites. Tissue is homogenized in the presence of RNA Bee (Tel-Test, Inc., Friendswood, Tx.), followed by chloroform extraction and ethanol precipitation to isolate total RNA. Total RNA is further purified using an RNeasy mini kit (Qiagen GmbH, Hilden, Germany) according to the manufacturer's instructions, and serves as the template for first strand cDNA synthesis. Reverse transcription is primed using five primers (in five separate reactions) that hybridize to sequences within the human immunoglobulin (Ig) constant (C) region genes (the CMu.2, CG, CA.3, CK.2 and CL.2 primers) and is performed with rTth DNA polymerase (Applied Biosystems, Foster City, Calif.) in the presence of manganese acetate according to manufacturer's instructions. The sequence of the five primers or primer sets is as follows: CMu.2 (5′ TCCTGTGCGAGGCAGCCAACG 3′, SEQ ID NO:35), CG (5′ GCCTGAGTT CCACGACACCGTCAC 3′, SEQ ID NO:36), CA.3 (5′ TGTCCGCT TTCGCTCCAGGTC 3′, SEQ ID NO:37), CK.2 (5° CCACTGTATTTTGGCCT CTCTGGGATAGAAGTT 3′, SEQ ID NO:38, and CL.2 (5′ GCTCCCGGGTAGAA GTCACT 3′, SEQ ID NO:39). The resultant cDNA is further purified using a QIAquick PCR purification kit (Qiagen GmbH, Hilden, Germany) according to manufacturer's instructions.

Using the purified first strand cDNA as template, anchor PCR is carried out to identify which V regions are utilized for expression of the immunoglobulin heavy and light chains in the tumor sample. The procedure involves dGTP tailing of the 1^{st} strand cDNA with terminal transferase (TdT) (Roche Applied Science, Indianapolis, Ind.) in the presence of cobalt chloride according to manufacturer's instructions with the exception that instead of using the supplied Roche 5× reaction buffer, the 5× rTdT Buffer from USB Corp. (Cleveland, Ohio) is used. The polyG tailed cDNA is then purified using a QIAquick PCR purification kit (Qiagen GmbH, Hilden, Germany) according to manufacturer's instructions.

Purified polyG tailed cDNA is then PCR amplified with primer An10cvH (5′ TCTA GAATTCACGCGTCCCCCCCCCC 3′, SEQ ID NO:40) and An12cvH (5′ TCTAGAAT TCACGCGTCCCCCCCCCCCC 3′, SEQ ID NO:41), in separate reactions, as the forward primers and the appropriate constant primer (CMu.3, CG.2, CA.4, CK.6 or CL.5) as the reverse primer. The sequence of the constant primers is as follows: Cmu.3 (5′ CAACG GCCACGCTGCTCGTATCCG 3′ SEQ ID NO:42), CG.2 (5′ GTAGTCCT TGACCAGGCAGCCCAG 3′, SEQ ID NO:43), CA.4 (5′ GGCTCCTGGGGG AAGAAGCCC 3′, SEQ ID NO:44), CK.6 (5′ GAAGTTATTCAGCAGGCACACAA CAGAGGC 3′, SEQ ID NO:45), and CL.5 (5° CACACCAGTGTGGCCTTGTTGGCTTG 3′, SEQ ID NO:46). PCR amplifications are performed with Pfu DNA polymerase (Stratagene, San Diego, Calif.) according to the manufacturer's instructions for 30 cycles using the following profile: 94° C. for 40 seconds; 63° C. for 40 seconds; and 72° C. for 80 seconds.

Amplification products are then electrophoresed on a 1.8% agarose TAE gel and excised for further purification. Anchor PCR products from An10cvH and An12cvH are combined for each of the five constants chains (CMu, CG, CA, CK, and CL) prior to purification, resulting in 5 distinct amplification products. Combined products are purified using a QIAquick Gel Extraction kit (Qiagen GmbH, Hilden, Germany) according to manufacturer's instructions. Each product is then ligated into pCR4Blunt-TOPO vector (Invitrogen, Carlsbad, Calif.) and transformed into E. coli using a Zero Blunt TOPO PCR Cloning Kit For Sequencing with One Shot TOP10 Chemically Competent E. coli (Invitrogen, Carlsbad, Calif.) according to the manufacturer's instructions. Each transformation is then plated onto two LB agar+100 μg/ml carbenicillin plates and incubated overnight at 37° C. 24 colonies are then picked, archived on a LB agar+100 μg/ml carbenicillin grid plate and screened by PCR for each of the 5 constant chains. PCR screening reactions are performed with AmpliTaq DNA polymerase (Applied Biosystems, Foster City, Calif.) using An8cvH forward primer (5′ TCTAGAATTCACGCGTCCCCCCCC 3′, SEQ ID NO:46) and the appropriate constant primer (CMu.3, CG.2, CA.4, CK.6 or CL.5) according to the manufacturer's instructions using the following profile:initial denaturation cycle of 94° C. for 5 minutes followed by 30 cycles of:94° C. for 20 seconds, 63° C. for 20 seconds, and 72° C. for 80 seconds.

The PCR screening products then serve as template for DNA sequencing. DNA sequencing is performed with 1 μl of PCR product and the appropriate constant primer CMu (5′ GGGGAAAAGGGTTGGGGCGGATGC 3′, SEQ ID NO:47); CG.2, CA (5′ AGGCTCA GCGGGAAGACCTTG 3′, SEQ ID NO:48); CK (5′ GGTTCCGGACTTAAGCTGCTCA TCAGATGGCGGG 3′, SEQ ID:49) or CL (5′ GGCGCCGCCTTGGGCTGACCT AGGACGGT 3′, SEQ ID NO:50, using Big Dye Terminator v3.1 Cycle Sequencing kit (Applied Biosystems, Foster City, Calif.) according to manufacturer's instructions using the following thermal cycling profile:initial denaturation cycle of 96° C. for 1 minute followed by 25 cycles of:96° C. for 10 seconds, 50° C. for 5 seconds, and 60° C. for 60 seconds.

Cycle sequencing reactions are then subjected to ethanol precipitation in the presence of sodium acetate, dried, and resuspended in 20 μl of Hi-Di Formamide (Applied Biosystems, Foster City, Calif.). Reactions are then denatured at 95° C. for 5 min and loaded onto an ABI Prism 3100 Genetic Analyzer (Applied Biosystems, Foster City, Calif.) and subjected to capillary electrophoresis according to manufacturer's instructions. Sequence data is visualized using the Lasergene Software Suite (DNASTAR, Inc., Madison Wis.). Tumor-derived sequence is determined statistically. For example, if the tumor is expressing a kappa light chain, then all 24 of the lambda anchor clones will have a unique sequence, whereas 12 of the kappa clones will have unique sequence and 12 will have the identical sequence (i.e., half of the biopsy cells expressing a kappa light chain are normal, and half are tumor). The absolute ratio of normal to tumor cell is biopsy specific.

Once the sequences of the tumor-derived heavy and light chains have been determined, they can then be assigned a subgroup family. The International Immunogenetics Information System web site, which is currently http:, followed by //imgt.cines.fr, contains a database of all germline immunoglobulin sequences and their subgroup family designation. Performing a BLAST Software (National Center for Biotechnology Information, Bethesda Md.) analysis comparing tumor-derived sequences to the germline sequence database will identify the germline sequence which most closely matches the input tumor sequence for each chain. The germline sequence that produces the best match will have the highest Score (Bits) value and the lowest E value. Subgroup family assignments for tumor-derived sequences correspond to the germline subgroup assignment of this best match sequence using the default parameter of the nucleotide-nucleotide BLAST Software (wwwblast-20040725-ppc32-macosx version 2.2.9+). Performing such an analysis on the patient population resulted in the data shown in Table 1 below:

TABLE 1A

| | | Family | Num | Percent | Percent | Percent |
|---|---|---|---|---|---|---|
| Chain | Family | Member | Pt | Family | Chain | Total |
| H | | | 559 | | | 100 |
| | IGHV1 | | 36 | | 6.4 | 6.4 |
| | | 1-2 | 5 | 13.9 | 0.9 | 0.9 |
| | | 1-3 | 3 | 8.3 | 0.5 | 0.5 |
| | | 1-8 | 4 | 11.1 | 0.7 | 0.7 |
| | | 1-18 | 13 | 36.1 | 2.3 | 2.3 |
| | | 1-46 | 6 | 16.7 | 1.1 | 1.1 |
| | | 1-69 | 5 | 13.9 | 0.9 | 0.9 |
| | IGHV2 | | 6 | | 1.1 | 1.1 |
| | | 2-5 | 4 | 66.7 | 0.7 | 0.7 |
| | | 2-26 | 1 | 16.7 | 0.2 | 0.2 |
| | | 2-70 | 1 | 16.7 | 0.2 | 0.2 |
| | IGHV3 | | 373 | | 66.7 | 66.7 |
| | | 3-7 | 41 | 11 | 7.3 | 7.3 |
| | | 3-9 | 3 | 0.8 | 0.5 | 0.5 |
| | | 3-11 | 33 | 8.8 | 5.9 | 5.9 |
| | | 3-15 | 23 | 6.2 | 4.1 | 4.1 |
| | | 3-21 | 24 | 6.4 | 4.3 | 4.3 |
| | | 3-23 | 95 | 25.5 | 17 | 17 |
| | | 3-30 | 26 | 7 | 4.7 | 4.7 |
| | | 3-33 | 5 | 1.3 | 0.9 | 0.9 |
| | | 3-48 | 76 | 20.4 | 13.6 | 13.6 |
| | | 3-49 | 2 | 0.5 | 0.4 | 0.4 |
| | | 3-53 | 15 | 4 | 2.7 | 2.7 |
| | | 3-66 | 8 | 2.1 | 1.4 | 1.4 |
| | | 3-72 | 1 | 0.3 | 0.2 | 0.2 |
| | | 3-73 | 5 | 1.3 | 0.9 | 0.9 |
| | | 3-74 | 16 | 4.3 | 2.9 | 2.9 |
| | IGHV4 | | 131 | | 23.4 | 23.4 |
| | | 4-4 | 9 | 6.9 | 1.6 | 1.6 |
| | | 4-30 | 5 | 3.8 | 0.9 | 0.9 |
| | | 4-31 | 6 | 4.6 | 1.1 | 1.1 |
| | | 4-34 | 34 | 26 | 6.1 | 6.1 |
| | | 4-39 | 30 | 22.9 | 5.4 | 5.4 |
| | | 4-55 | 2 | 1.5 | 0.4 | 0.4 |
| | | 4-59 | 31 | 23.7 | 5.5 | 5.5 |
| | | 4-61 | 10 | 7.6 | 1.8 | 1.8 |
| | | 4-b | 4 | 3.1 | 0.7 | 0.7 |
| | IGHV5 | | 9 | | 1.6 | 1.6 |
| | | 5-51 | 7 | 77.8 | 1.3 | 1.3 |
| | | 5-a | 2 | 22.2 | 0.4 | 0.4 |
| | IGHV6 | | 2 | | 0.4 | 0.4 |
| | | 6-1 | 2 | 100 | 0.4 | 0.4 |
| | IGHV7 | | 2 | | 0.4 | 0.4 |
| | | 7-4 | 2 | 100 | 0.4 | 0.4 |

TABLE 1B

| | | Family | Num | Percent | Percent | Percent |
|---|---|---|---|---|---|---|
| Chain | Family | Member | Pt | Family | Chain | Total |
| K | | | 329 | | | 58.9 |
| | IGKV1 | | 113 | | 34.3 | 20.2 |
| | | 1-5 | 33 | 29.2 | 10 | 5.9 |
| | | 1-6 | 4 | 3.5 | 1.2 | 0.7 |
| | | 1-8/1D-8 | 1 | 0.9 | 0.3 | 0.2 |
| | | 1-9 | 9 | 8 | 2.7 | 1.6 |
| | | 1-12/1D-12 | 8 | 7.1 | 2.4 | 1.4 |
| | | 1-16/1D-16 | 2 | 1.8 | 0.6 | 0.4 |
| | | 1-17/1D-17 | 12 | 10.6 | 3.6 | 2.1 |
| | | 1-27/1D-27 | 7 | 6.2 | 2.1 | 1.3 |
| | | 1-33/1D-33 | 3 | 2.7 | 0.9 | 0.5 |
| | | 1-39/1D-39 | 34 | 30.1 | 10.3 | 6.1 |
| | IGKV2 | | 31 | | 9.4 | 5.5 |
| | | 2-24/2D-24 | 4 | 12.9 | 1.2 | 0.7 |
| | | 2-28/2D-28 | 14 | 45.2 | 4.3 | 2.5 |
| | | 2-29/2D-29 | 2 | 6.5 | 0.6 | 0.4 |
| | | 2-30/2D-30 | 11 | 35.5 | 3.3 | 2 |
| | IGKV3 | | 112 | | 34 | 20 |
| | | 3-11/3D-11 | 26 | 23.2 | 7.9 | 4.7 |
| | | 3-15/3D-15 | 22 | 19.6 | 6.7 | 3.9 |
| | | 3-20/3D-20 | 64 | 57.1 | 19.5 | 11.4 |
| | IGKV4 | | 68 | | 20.7 | 12.2 |
| | | 4-1 | 68 | 100 | 20.7 | 12.2 |
| | IGKV6 | | 5 | | 1.5 | 0.9 |
| | | 6-21/6D-21 | 5 | 100 | 1.5 | 0.9 |

TABLE 1C

| | | Family | Num | Percent | Percent | Percent |
|---|---|---|---|---|---|---|
| Chain | Family | Member | Pt | Family | Chain | Total |
| L | | | 234 | | | 41.9 |
| | IGLV1 | | 101 | | 43.2 | 18.1 |
| | | 1-36 | 1 | 1 | 0.4 | 0.2 |
| | | 1-40 | 25 | 24.8 | 10.7 | 4.5 |
| | | 1-44 | 22 | 21.8 | 9.4 | 3.9 |
| | | 1-47 | 8 | 7.9 | 3.4 | 1.4 |
| | | 1-51 | 45 | 44.6 | 19.2 | 8.1 |
| | IGLV2 | | 55 | | 23.5 | 9.8 |
| | | 2-8 | 17 | 30.9 | 7.3 | 3 |
| | | 2-11 | 9 | 16.4 | 3.8 | 1.6 |
| | | 2-14 | 17 | 30.9 | 7.3 | 3 |
| | | 2-18 | 1 | 1.8 | 0.4 | 0.2 |
| | | 2-23 | 11 | 20 | 4.7 | 2 |
| | IGLV3 | | 35 | | 15 | 6.3 |
| | | 3-1 | 2 | 5.7 | 0.9 | 0.4 |
| | | 3-9 | 2 | 5.7 | 0.9 | 0.4 |
| | | 3-10 | 6 | 17.1 | 2.6 | 1.1 |
| | | 3-19 | 11 | 31.4 | 4.7 | 2 |
| | | 3-21 | 6 | 17.1 | 2.6 | 1.1 |
| | | 3-25 | 8 | 22.9 | 3.4 | 1.4 |
| | IGLV4 | | 19 | | 8.1 | 3.4 |
| | | 4-3 | 1 | 5.3 | 0.4 | 0.2 |
| | | 4-69 | 18 | 94.7 | 7.7 | 3.2 |
| | IGLV5 | | 2 | | 0.9 | 0.4 |
| | | 5-39 | 1 | 50 | 0.4 | 0.2 |
| | | 5-52 | 1 | 50 | 0.4 | 0.2 |
| | IGLV6 | | 2 | | 0.9 | 0.4 |
| | | 6-57 | 2 | 100 | 0.9 | 0.4 |
| | IGLV7 | | 11 | | 4.7 | 2 |
| | | 7-43 | 6 | 54.5 | 2.6 | 1.1 |
| | | 7-46 | 5 | 45.5 | 2.1 | 0.9 |
| | IGLV8 | | 3 | | 1.3 | 0.5 |
| | | 8-61 | 3 | 100 | 1.3 | 0.5 |
| | IGLV9 | | 1 | | 0.4 | 0.2 |
| | | 9-49 | 1 | 100 | 0.4 | 0.2 |
| | IGLV10 | | 5 | | 2.1 | 0.9 |
| | | 10-54 | 5 | 100 | 2.1 | 0.9 |

Similar to a normal B cell population, the Non-Hodgkin's B Cell lymphoma patient population screened as described above does not utilize all known V region genes at the same frequency. The results in Table 1 show a skewed representation of gene usage with some families and family members being more frequently expressed than others. The most highly expressed (e.g. those found in more than 5% of the population) are shown in bold in Table 1. Importantly, it is noted, according to Table 1, that mAbs generated against HV3-23 could recognize up to 16% of the patient population and mAbs against KV4-1 up to 12%.

### EXAMPLE 2

### Family Member- and Family-Specific mAbs

This example describes the creation of family-specific LV1, LV2, KV1 and HV4 mAbs and family member specific KV4-1, HV3-23, LV2-8, KV3-11 and KV1-5 mAbs. In particular, this example describes methods used to generate one LV1 reactive clone (20H5), nine LV2 reactive clones (6D7, 15E8, 19A11, 7H7, 13H10, 2C6, 2E6, 9E3, and 20C1), eleven KV4-1 reactive clones (15E1, 1E10, 1F10, 1G10, 6G2/6G7, 5G10, 10E7, 10H7 19C5, 20G11, and 7G3), two KV4-1+KV3 reactive clones (11H8 and 12C3), one KV4-1+KV1-9 reactive clone (9C2), eight HV3-23 reactive clones (10D6, 13F5, 1A3, 1E9, 2H10, 3C9, 6C9-F3, and 6D9), two LV2-8 reactive clones (12E9 and 11G3), two LV2+LV3-25 reactive clones (4A6/A10 and 4H11), one HV4 reactive clone (15H5), one KV3-11 reactive clone (6B6), six KV1-5 reactive clones (2A6, 9G11, 12F10, 16A12, 17D9, and 21E9), eight KV1 reactive clones (3F3, 10A6, 12B9, 12H12, 24D3, 25G7, 29F1, and 30A7) and one KV1+KV6-21 reactive clone (9C5).

A. Materials and Methods

Immunogen Forms and Purification

The V regions (HV, KV and LV) are all human derived (from the pool of over 500 NHL patient Id proteins) and use a corresponding constant region (HC, KC, and LC) for protein expression. Six expression vectors have been prepared, each of the three constant regions (HC, KC, and LC) were constructed from both human and mouse. Each recombinant Id protein contains two identical heavy and two identical light chain molecules. Mouse BW5147.G.1.4 cells (ATCC CRL-1588) are transfected by electroporation for expression of fully human or chimeric Id proteins. To construct a fully human Id protein, the HV is cloned into a human HC isotype G3 (HCG3) expression vector and the KV or LV into either a human KC, or LC expression vector. Likewise to generate human-mouse chimeric Id proteins the mouse HC isotype G2a (HCG2a) is paired with a KV or LV cloned into mouse KC, or LC. From the six expression vectors, three forms of Id proteins were prepared, each containing patient derived V regions cloned into the respective constant region expression vector, either human or mouse. The three forms of immunogen are the fully human and the human-mouse chimeras in which both V regions are from the same patient or a human-mouse chimera from that has two different patient V regions.

Id protein secreted into the media supernatant is purified using Protein G Sepharose and the eluate is dialyzed against 0.9% saline. Purified Id protein can be conjugated to KLH or remain unconjugated.

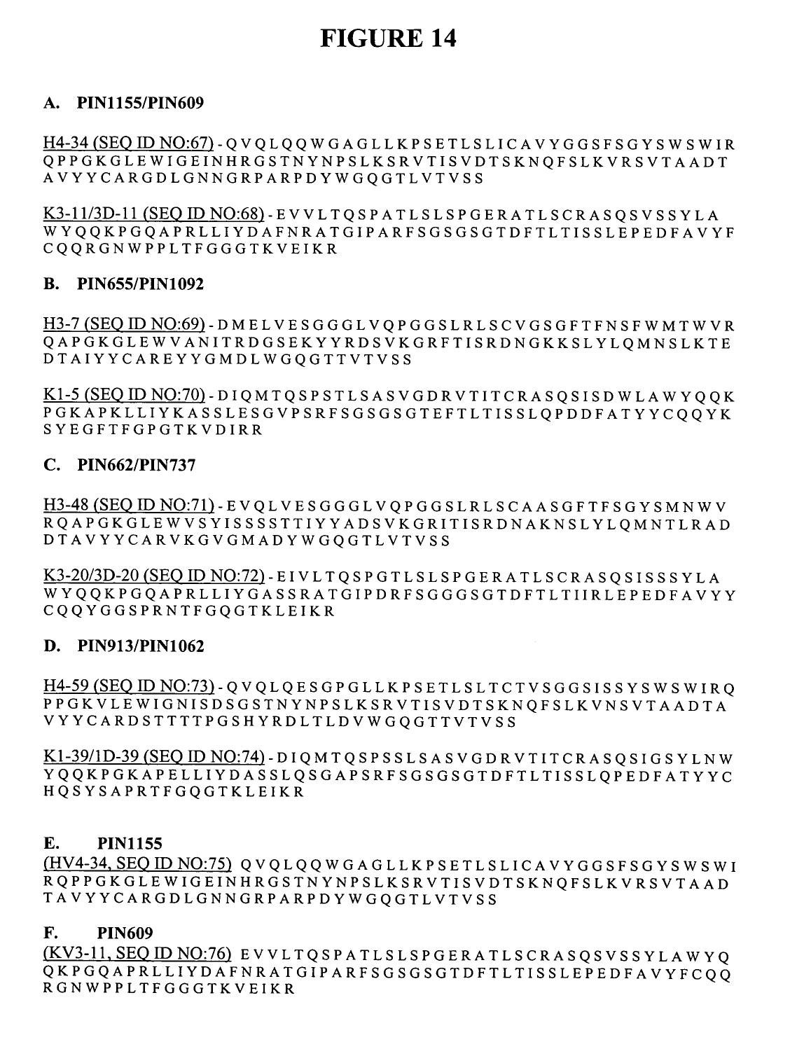

The 18 relevant patient derived V region immunogens, come from 13 different patient Id proteins. The amino acid sequence for 10 of these 18 V regions is shown in FIG. 1 as follows: A) PIN574, composed of HV4-39 (SEQ ID NO:1) and LV1-40 (SEQ ID NO:2); B) PIN149, composed of HV3-23 (SEQ ID NO:3) and KV4-1 (SEQ ID NO:4); C) PIN 116, composed of HV1-46 (SEQ ID NO:5) and LV2-8 (SEQ ID NO:6); D) PIN647 composed of HV3-48 (SEQ ID NO:7) and LV2-14 (SEQ ID NO:8); and E) PIN628 composed of HV3-7 (SEQ ID NO:9) and KV4-1 (SEQ ID NO:10). The amino acid sequence for 8 of these 18 V regions is shown in FIG. 14 as follows: A) PIN 1155 HV4-34 (SEQ ID NO:67) and PIN609 KV3-11 (SEQ ID NO:68); B) PIN655 HV3-7 (SEQ ID NO:69) and PIN1092 KV1-5 (SEQ ID NO:70); C) PIN662 HV3-48 (SEQ ID NO:71) and PIN737 KV3-20 (SEQ ID NO:72); D) PIN913 HV4-59 (SEQ ID NO:73) and PIN1062 KV1-39 (SEQ ID NO:74).

Immunizations and Fusions

Animals are primed, with immunogen vortexed in complete Syntex Adjuvant Formulation-1 (cSAF-1) and peptide (Ac-muramly-Thr-D-Glu-NH2) or emulsified in Complete Freund's adjuvant (CFA), either subcutaneously (SC) or intraperitoneally (IP). When boosted, mice are injected SC with incomplete SAF-1 (iSAF-1) or Incomplete Freund's Adjuvant (IFA) up to 4 times. To generate B cell blasts a pre-fusion injection was given either IP or intravenously (IV) in saline. For fusions 1-5, 7-11, and 12-25, injections occurred every 14 days except for the pre-fusion boost that occurred three days prior to fusion. Fusions 6 and 12 tested a short immunization protocol; mice were immunized IP on day zero and give an IV pre-fusion boost on day seven. The 25 fusions described in this example are presented in Table 2.

TABLE 2

| | | | | | | | | | | |
|---|---|---|---|---|---|---|---|---|---|---|
| | | | | | | | | Route of | | No. of |
| | | Immunogen | | | Immunogen | | Adjuvant | immunization | Total | spleens |
| Fusion | Study | Pt. V | HV | LV | CH | | (prime/boost/ | and number | number of | in |
| Number | Number | regions | region | region | region | KLH | pre-fusion) | of injections | injections | fusion |

GROUP 1 CCM-1

| | | | | | | | | | | |
|---|---|---|---|---|---|---|---|---|---|---|
| 1 | NV#2 | 149 | 3-23 | K4-1 | mouse | no | CFA/IFA/saline | SCx5/IP | 6 | 1 |
| 2 | NV#6 | 149 | 3-23 | K4-1 | mouse | no | CFA/IFA/saline | SCx5/IP | 6 | 1 |
| 3 | SN12#16 | 149 | 3-23 | K4-1 | mouse | yes | cSAF/iSAF/saline | SCx3/IP | 4 | 1 |
| 4 | SN12#2 | 149 | 3-23 | K4-1 | mouse | no | cSAF/iSAF/saline | SCx3/IP | 4 | 1 |
| 5 | SN14 | 149 | 3-23 | K4-1 | human | no | cSAF/iSAF/saline | SCx3/IP | 4 | 2 |
| 6 | SN15 | 149 | 3-23 | K4-1 | human | no | CFA/saline | IPx1/IV | 2 | 3 |
| 7 | SN16 | 149 | 3-23 | K4-1 | human | no | cSAF/iSAF/saline | SCx1/IP | 2 | 3 |
| 8 | SN17 | 149 | 3-23 | K4-1 | human | no | cSAF/iSAF/saline | SCx2/IP | 3 | 3 |
| 9 | SN18 | 610 | 3-23 | K4-1 | human | no | cSAF/iSAF/saline | SCx3/IP | 4 | 3 |

GROUP 2 ProCHO5

| | | | | | | | | | | |
|---|---|---|---|---|---|---|---|---|---|---|
| 10 | SN19 | 574 | 4-39 | L1-40 | human | no | cSAF/iSAF/saline | SCx3/IP | 4 | 3 |
| 11 | SN20 | 149 | 3-23 | K4-1 | human | no | cSAF/iSAF/saline | SCx3/IP | 4 | 3 |
| 12 | SN21 | 149 | 3-23 | K4-1 | mouse | yes | CFA/saline | IPx1/IV | 2 | 3 |
| 13 | SN23 | 149 | 3-23 | K4-1 | mouse | yes | cSAF/iSAF/saline | SCx3/IP | 4 | 3 |
| 14 | SN24 | 149 | 3-23 | K4-1 | mouse | yes | cSAF/iSAF/saline | SCx4/IP | 5 | 2 |
| 15 | SN26 | 116 | 1-46 | L2-8 | human | yes | cSAF/iSAF/saline | SCx3/IP | 4 | 3 |
| 16 | SN27 | 116 | 1-46 | L2-8 | human | no | cSAF/iSAF/saline | SCx3/IP | 4 | 3 |
| 17 | SN28 | 647 | 3-48 | L2-14 | human | yes | cSAF/iSAF/saline | SCx3/IP | 4 | 4 |
| 18 | SN29 | 116 | 1-46 | L2-8 | human | yes* | cSAF/iSAF/IFA/saline | SCx3/IP | 4 | 4 |
| 19 | SN30 | 201 | 5-51 | K1-39 | human | yes* | CFA/IFA/saline | SCx3/IP | 4 | 4 |
| 20 | SN31 | 628 | 3-7 | K4-1 | human | yes* | CFA/IFA/saline | SCx3/IP | 4 | 3 + 3 |
| 21 | SN32 | 628 | 3-7 | K4-1 | mouse | yes* | CFA/IFA/saline | SCx3/IP | 4 | 3 + 3 |
| n/a | SN33 | 607/149 | 3-48 | K4-1 | human | yes* | CFA/IFA | SCx3/IP | 4 | 0 |
| 22 | SN34 | 1155/609 | 4-34 | K3-11 | mouse | yes* | cSAF/iSAF/saline | SCx3/IP | 4 | 1 + 2 |
| 23 | SN35 | 655/1092 | 3-7 | K1-5 | mouse | yes* | cSAF/iSAF/saline | SCx3/IP | 4 | 0 + 3 |
| 24 | SN36 | 662/737 | 3-48 | K3-20 | mouse | yes* | cSAF/iSAF/saline | SCx3/IP | 4 | 1 + 2 |
| 25 | SN37 | 913/1062 | 4-59 | K1-39 | mouse | yes* | cSAF/iSAF/saline | SCx3/IP | 4 | 0 + 3 |

The first 4 fusions were performed with cells from a single spleen, fusions 5 and 14 with cells from two spleens, fusions 6-13 and 15-16 with cells from three spleens, and two fusions with two spleens for each fusion for fusions 17-19. Beginning with fusion 20, in addition to BALB/C, a second strain of mouse (C3H-HeN) has been employed. Only spleens from one mouse strain are fused but two different mouse strains immunized with the same immunogen can be tested in one fusion set. For each strain all three spleens were employed (for a total of six mice) for fusions 21 and 21. There was no fusion for SN33 (see antisera screening). Fusions 22 and 24 were performed with one BALB/C spleen and two C3H-HeN and all three C3H-HeN spleens were used in fusions 23 and 25. Mouse splenocytes and mouse B cell fusion partner Fox-NY (ATCC CRL-1732) were fused using a standard polyethylene glycol centrifugation method. Fused cells were seeded in 96-well plates ranging from 0.5 to 3.0×10⁵/well. Fox-NY cells that do not acquire hypoxanthine phosphoribosyl transferase from spleen cells die in hypoxanthine/aminopterin/thymidine (or azaserine, see fusion 23) selection medium.

Primary and Secondary Hybridoma Supernatant Screening

Hybridoma supernatants were screened using an ELISA to measure binding to the Id protein V region. The fully human, unconjugated form of the Id protein was used. Primary screen 1 was performed on the parent hybridoma plates during week two following the fusion, day 8-14 post-fusion. Beginning with fusion 5, when reactive clone numbers were low in the the primary screen, a second screening was added for all plates to be done day 14-21, called primary screen 2.

The form of the immunogen dictates the primary screening protocol. Originally the primary screen of hybridoma supernatants included, in addition to the immunogen, an Id protein derived from a V region from a different HV region family and the alternate light chain constant region (lambda if kappa-immunized). By including an additional Id protein, one can identify hybridomas that are specific for the HC region because all HC regions are the same isotype, HCG3. In fusions 5-8, for example, all hybridoma supernatants were screened against the immunogen Id protein PIN149, HV3-23 (HCG3) and KV4-1 (KC), and against the additional Id protein PIN116, HV1-46 (HCG3) and LV2-8 (LC). In this example the HC-specific clones would be screened positive for both PIN149 and PIN116. It is not possible to identify anti-KC region mAb in the primary screen, this is done before the specificity screen (see below). Beginning with fusion 17, the additional Id was eliminated in the primary screening and combined with the screen to identify anti-KC or -LV and anti-Id reactive clones. Hybridoma supernatants from mice immunized with chimeric Id in which both V regions came from the same patient were only tested on one fully human form of the Id protein, whereas when two different patient V regions were used to generate the chimeric Id, both fully human Id proteins were typically tested.

Cloning, Expanding and Freezing

As cell growth permits, cells from wells screened positive in the primary screens were transferred from 96-well to 24-well plates and expanded for re-screening and freezing. If necessary, the single antibody-producing clones of interest were isolated by limiting dilution plating. Hybridomas were plated at two dilutions 3.0 and 0.3 cells/well. Cloning was considered successful if less than or equal to one cell is plated in every 3^{rd} well (30% cell growth/96-well plate). From the 24-well plate, a clonal population of cells were expanded to a T-25 flask. One vial of cells was frozen from the T-25 flask for a stock and the supernatant was used to do the specificity screening.

Screening for V Region Family Member-and Family-Specific mAbs.

Before specificity screening, all of the clonal immunogen-positive hybridoma supernatants were re-screened on the immunogen, a non-family member Id protein, and KLH, if animals were immunized with Id protein that has been conjugated KLH. As described above, this screen identifies HC-specific clones but not mAbs against the light chain constant region. Clones identified positive to the immunogen and KLH were cloned and rescreened. Those found positive to the immunogen but not to the non-family member Id protein or KLH were tittered on the immunogen. In addition to normalizing for different binding affinities and concentrations, each hybridoma supernatant was titered on the fully human form of the Id protein used as the immunogen(s) for the V regions. The titer or dilution resulting in an ELISA absorbance of 2.5 to 3 OD after 30 min. incubation time was then used. Hybridoma specificity was determined by screening against available Id proteins expressing the same HV and KV or LV as well as different HV, KV and LV. The final ELISA results are expressed as follows: 0 (<0.5 OD), 1 (0.5-1.0 OD), or 4 (1-4 OD). Positive hybridoma supernatants are categorized according to specificity: anti-Id, anti-constant region (HC, KC or LC), or V region (HV, KV, LV family or family member).

ELISA Protocol

In general, the fully human Id protein or proteins containing the immunogen V regions were coated onto 96-well ELISA plates at 2.5-5 ug/mL in carbonate buffer pH 9.6 and incubated at 4° C. for up to 14 days. On the day of the ELISA, plates were brought to RT and blocked with Tris-Tween-20, pH 7.6 for 15-60 min. at RT. Following a NaCl and Triton X-100 wash, diluted hybridoma cell culture supernatants were incubated overnight at 4° C. Hybridoma supernatant dilutions ranged from 1:2 to 1:25 and were prepared in PBS with 5% BSA. Plates were washed and incubated with HRP-conjugated goat anti-mouse IgG-specific detecting antibody. A chromogenic substrate (TMB) was used to measure the amount of mouse antibody bound to each well. The reaction was stopped at or before 30 minutes with IN H2SO4 and plates were read immediately. Absorbency readings at 450 nm ranging between 0-4 OD and using Molecular Devices plate reader and SOFTMAX PRO software. In general the primary screens from parent hybridoma supernatants were identified as positive if the signal was at least 2-fold over background (supernatant from wells without any cell growth). If the background was greater than 1 OD the samples were retested at a higher dilution. Supernatant background levels from the primary screen 1 differ and dilutions from 1:2 to 1:25 have been used to obtain a sensitive signal to noise reading for determining positive clones.

Antisera Screening

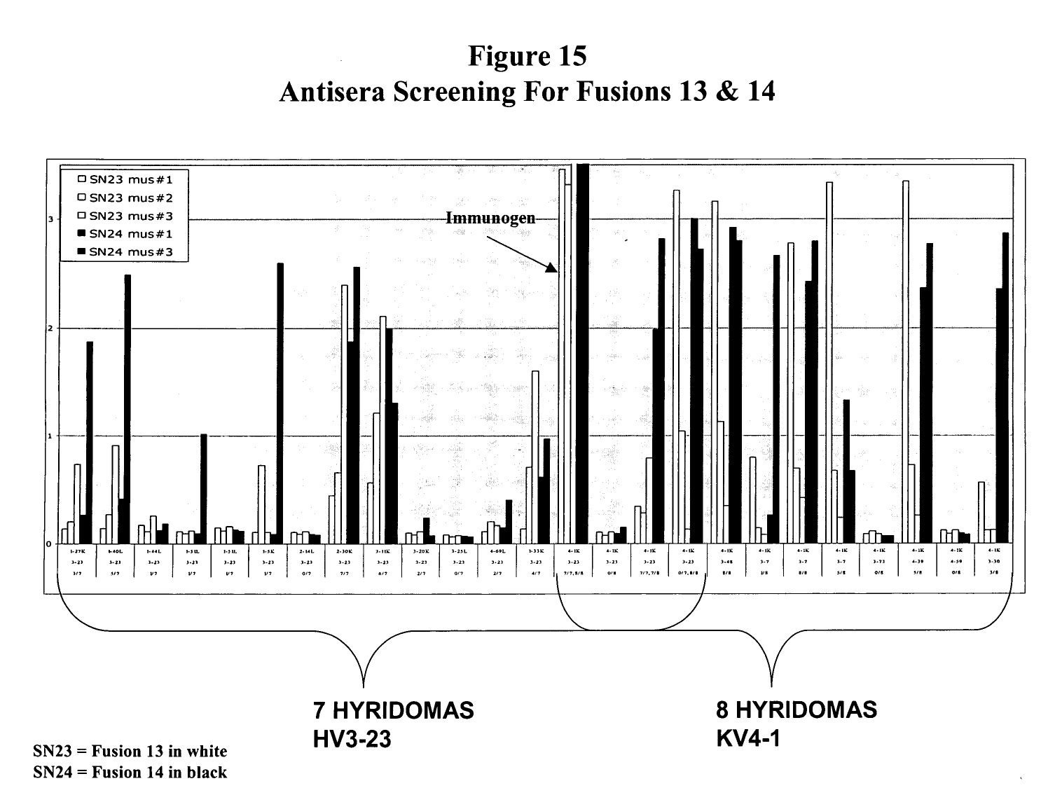

Using chimeric Id protein to immunize animals enables pre-screening the antisera prior to fusion. The pre-screens are useful for determining which animals are most likely to yield productive fusions. An antisera screen testing for immunoreactivity to several fully human Id proteins including immunogen, family member or family derived Id and non-family member derived Id shows the specificity of the polyclonal B cell response for individual animals. Differences in the polyclonal immune response among animals, strains, immunogens and immunization protocols can be observed in the intensity of the ELISA signal. Because this is a polyclonal response antisera screening does not necessarily reveal the exact specificity of any particular antibody, but does show the potential range of reactivity capable at the monoclonal level (see FIG. 15, 16, 17, 18, and 19 for antisera screening results are shown for fusions 12 and 13, 22, 23, 25, and SN33).

B. Fusion Results

Fusions 1-9 (purified from CCM-1 Media)

For Group 1, fusions 1-9, fully human and chimeric Id proteins were purified from CCM-1 media and therefore Id protein preparations contained some bovine IgG contamination. The 149-mG2a/mK chimera-expressing clone has a very low level of expression (˜0.8ug/mL) and therefore purified protein had a relatively higher level of bovine IgG contamination. Fusions from animals immunized with chimeric 149-mG2a/mK, Group 1, fusions 1, 2, 3 and 4 did not yield 149-specific mAbs. The mAbs characterized from these fusions were all reactive against bovine IgG-specific. This result concurs with early ELISA data screening mouse antisera on family member or family related and non-family member Id proteins. Due to its high expression level, the fully human Id protein had relatively little bovine IgG contamination. Four fusions, 5, 6, 7, and 8, from animals immunized with the fully human PIN149 Id protein, resulted in 219 reactive hybridomas. Of these, 14% were anti-Id, 6% anti-KC, 78% anti-HC and 0.9% (2 mAbs) were anti-HV3-23. These 2 mAbs recognized 13.9% (5 of 36) HV3-23-expressing Id proteins tested.

Fusion 9

Fusion 9 illustrates a comparison between two Id proteins that originated from the same heavy and light chain germline V regions, HV3-23/KV4-1, PIN610 (fusion 9) and PIN149 (fusion 5) Id proteins and the immunogenicity of bovine IgG. For removal of bovine IgG, PIN610 Id protein was subjective to an additional purification step. Following standard Protein G purification PIN610 Id protein was further purified with goat-anti-bovine IgG-coupled resin. Coomassie-stained SDS-PAGE analysis showed detectable levels of bovine IgG prior to but not after purification with the goat-anti-bovine IgG-coupled resin. ELISA results testing antisera from animals immunized with this two-stage purified PIN610 Id protein revealed immunoreactivity against PIN610 Id protein and against bovine IgG. This result demonstrates that even trace amounts (not detectable by Coomassie-stained SDS-PAGE) of some contaminants can be strongly immunogenic and likely reduce the probability of generating and finding V region family member- and family-specific mAbs. This fusion resulted in 24 immunogen reactive clones, three anti-Id, one KC- and 20 HC-specific mAbs. The specificity of the mAbs resulting from the two different HV3-23/KV4-1 Id proteins is very similar with the HC being the dominate epitope(s). Fusions 5-8 using PIN149 fully human Id protein and fusion 9 using PIN610 fully human Id protein resulted in 14% and 13% anti-Id, 6% and 4% anti-KC, 1% and 0% anti-HV, and 78% and 83% anti-HC mAbs respectively. This comparison and other fusions described below (see Fusions 13 and 14) led to the conclusion that using the fully human Id proteins as immunogens results in mostly anti-HC and anti-light chains mAbs.

Fusions 10-25 (purified from animal component free-media, ProCHO5)

Fusion 10

One V region family-specific mAb identified came from fusion 10 with Id protein from PIN574 that expresses HV4-39 (SEQ ID NO:1)/LV1-40 (SEQ ID NO:2). Two mAbs were screened positive and selected for further characterization. One mAb appears to be HCG3-specific. The other mAb, from clone 20H5, generated against PIN574 is LV1 family specific. There are 7 known LV1 families members: LV1-36, 1-40, 1-41, 1-44, 1-47, 1-50, and 1-51 and 18.1% of our NHL patient population screened express LV 1, as shown in Table 1. Hybridoma supernatant from clone 20H5 recognizes 47 of 57 (82%) LV1-expressing Id proteins tested, including 4 of the 5 LV1 family members found in our patient population (LV1-40, 1-51, 1-44, and 1-47), and zero of 20 the non-family member Id proteins tested.

Fusion 11

This fusion was performed to compare the influence of a bovine IgG as a contaminant in fusion 5. Both fusions use unconjugated PIN149 Id protein, SC×3/IP, and SAF for immunizing BALB/C mice. PIN149 Id protein is composed of H3-23 (SEQ ID NO:3) and K4-1 (SEQ ID NO:4). For fusion 11 Id protein was purified from ProCHO5 media (animal component free) whereas in fusion 5 Id protein was purified from CCM-1 media (containing bovine IgG) and shown to have about 5% bovine IgG contamination. One anti-Id mAb but no V-region family member- or family-specific mAbs were recovered from fusion 11.

Fusions 12, 13 and 14

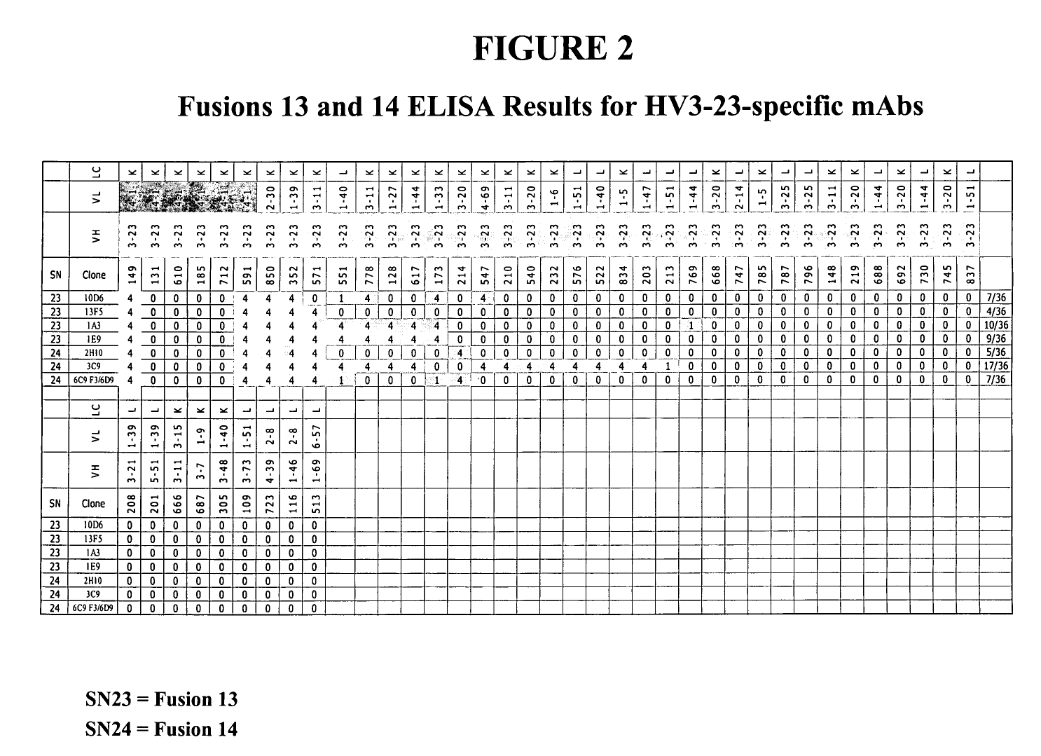

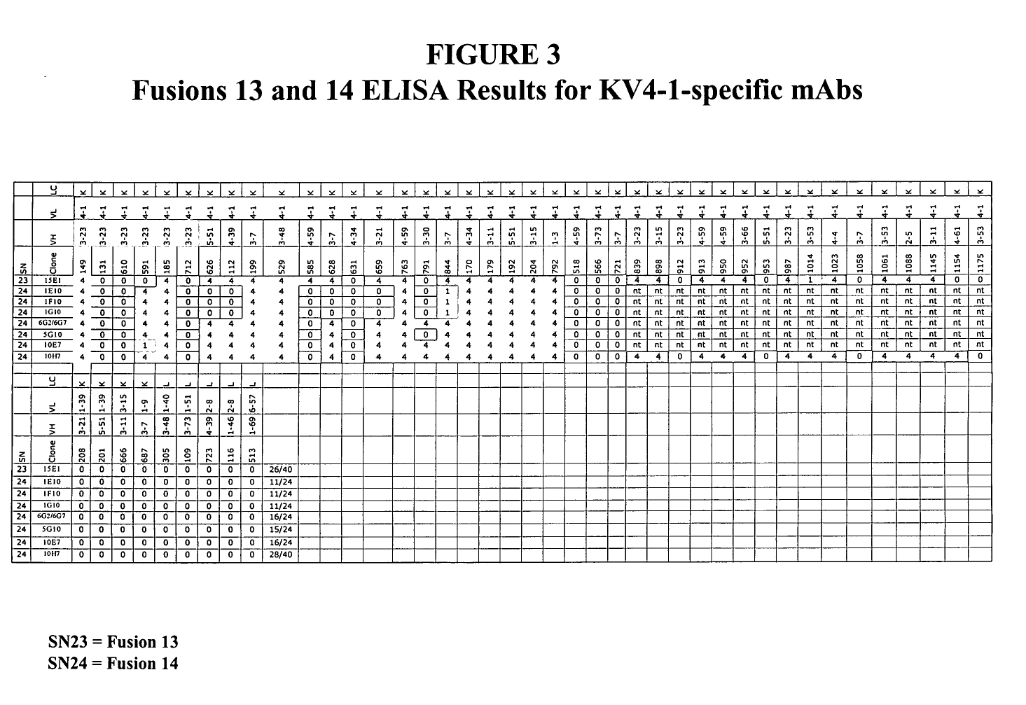

Fusions 12, 13, & 14 were implemented to re-test 149-mG2a/mK chimeric Id protein, purified from an animal component free media and to repeat the immunization comparisons done in fusions 5 and 6. Chimera 149-mG2a/mK was conjugated to KLH and an additional immunization protocol SC×4/IP was included. Fusions 13 and 14, from mice immunized with SC×3/IP & SC×4/IP respectively, resulted in 53 hybridomas with PIN149-specificity. Of these, 8 mAbs recognize HV3-23 (see Clone 10D6, 13F5, 1A3, 1E9, 2H10, 3C9, 6C9 F3, and 6D9 in FIG. 2). One of the 8, clones, 3C9, recognizes 17 of 36 (47%) Id proteins tested, while other clones recognize a smaller subset (4-10 of 36) of HV3-23 Id proteins. There are 5 mAb clones with similar recognition patterns that recognize 15-16 of 24 KV4-1 Id proteins tested (see Clones 15E1, 6G2/6G7, 5G10, 10E7, and 10H7 in FIG. 3). Three other KV4-1-specific mAbs recognize a subset of the same 16 Id proteins (see Clones 1E10, 1F10, and 1G10 in FIG. 3). Clones 15E1 and 10H7 were tested with an additional 16 KV4-1-expressing Id proteins increasing the screen to relevant 40 Id proteins. Clone 15E1 recognizes 26 of 40 or 65% and clone 10H7 recognizes 28 of 40 or 70% of KV4-1 Id proteins tested (see FIG. 3). These fusions support the conclusion from fusions 5-9 that the form of the immunogen is important for isolating V region family member- and family-specific mAbs. The dominant immunogenic epitopes in human HC and KC reduced the overall immunogenicity of the human HV and KV (see FIGS. 2 and 3 for mAb specificity and FIG. 15 antisera screening for fusions 13 and 14).

Fusion 15, 16, 17, & 18

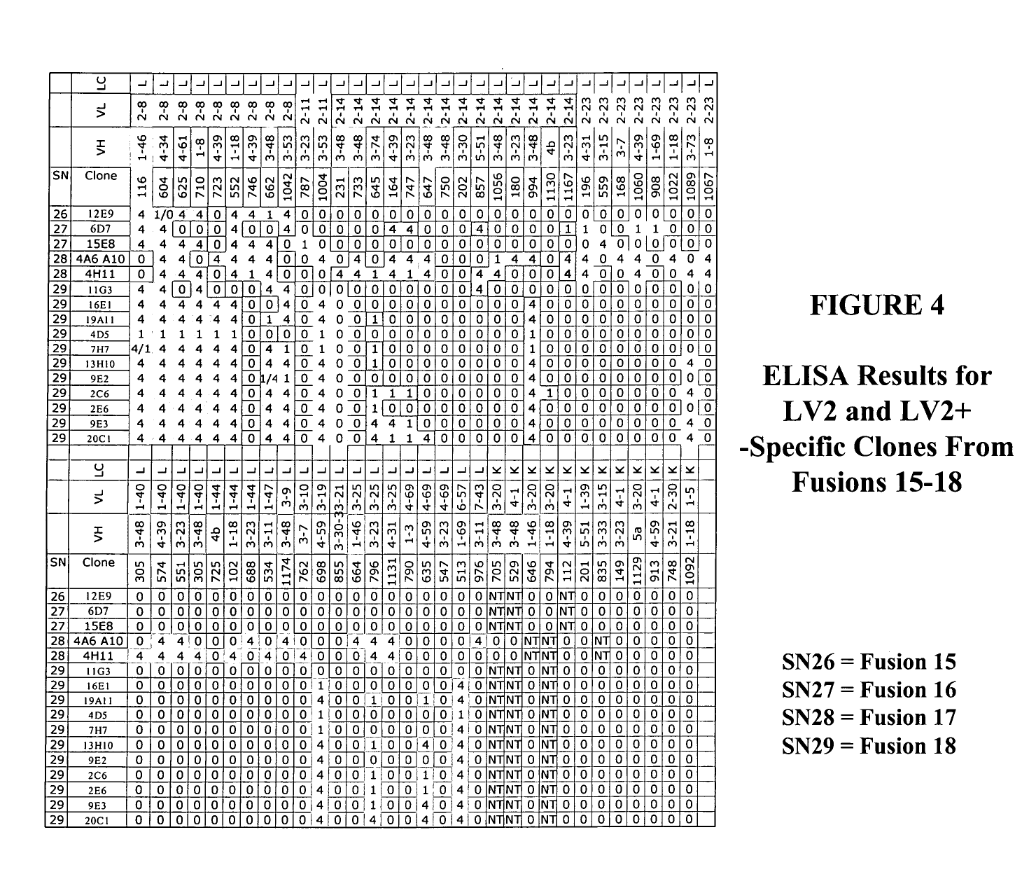

Two Id proteins were used to raise antibodies against the LV2 family, PIN116 (HV1-46 [SEQ ID NO:5] and LV2-8 [SEQ ID NO:6]) and PIN647 (HV3-48 [SEQ ID NO:7] and LV2-14 [SEQ ID NO:8]). Fusions 18 used different. Fusions 15 and 18 use the carrier molecule, KLH to modify the Id protein whereas fusion 16 is unconjugated. Fusion 15 resulted in two anti-Id and one LV2-8-specific clone (clone 12E9) that recognizes 6 of 7 LV-2-8 Id proteins tested (see FIG. 4). Fusion 16 resulted in two anti-LV2 clones one recognizing 10 of 32 and the other 8 of 32 LV2 family members (see clones 6D7 and 15E8 in FIG. 4). There were also 2 anti-LC clones from fusion 16. There were four LC-specific mAb from fusion 17 PIN647 Id protein (HV3-48, LV2-14) and two anti-LV2+-specific clones both of which have some cross-reactivity to LV1, LV3 and LV7 expressing Id proteins. Fusion 18 was the most productive fusion from immunizations with PIN116. Clones from fusion 18 include one anti-LV2 (Clone 11G3 recognizing 5 of 32 LV2) and ten anti-LV2 that also recognize a small subset of other LV family members (see Clones 16E1, 4D5, 9E2, 19A11, 7H7, 13H10, 2C6, 2E6, 9E3, and 20C1 in FIG. 4).

It appears that KLH influences the B cell response. Fully human PIN116 Id protein conjugated with KLH (fusion 18) resulted in about ten times more clones than the unconjugated PIN116 from fusion 16. These clones also have a different pattern of recognition although there are only 2 clones generated from PIN 116 Id protein conjugated to KLH.

Fusion 19

PIN201 (HV5-51, KV1-39) Id protein was conjugated to KLH and used as an immunogen. Fusion 19 did not result in V region family member- or family-specific mAbs.

Fusions 20 and 21

Fusion 20 utilized two strains of mice, BALB/C and C3H-HeN, that have different MHC class II haplotypes. Three mice from each strain were used in these fusions. The fusion results from these mice compares using the same V regions, PIN628 Id protein (HV3-7, KV4-1), and either the human or mouse constant regions.

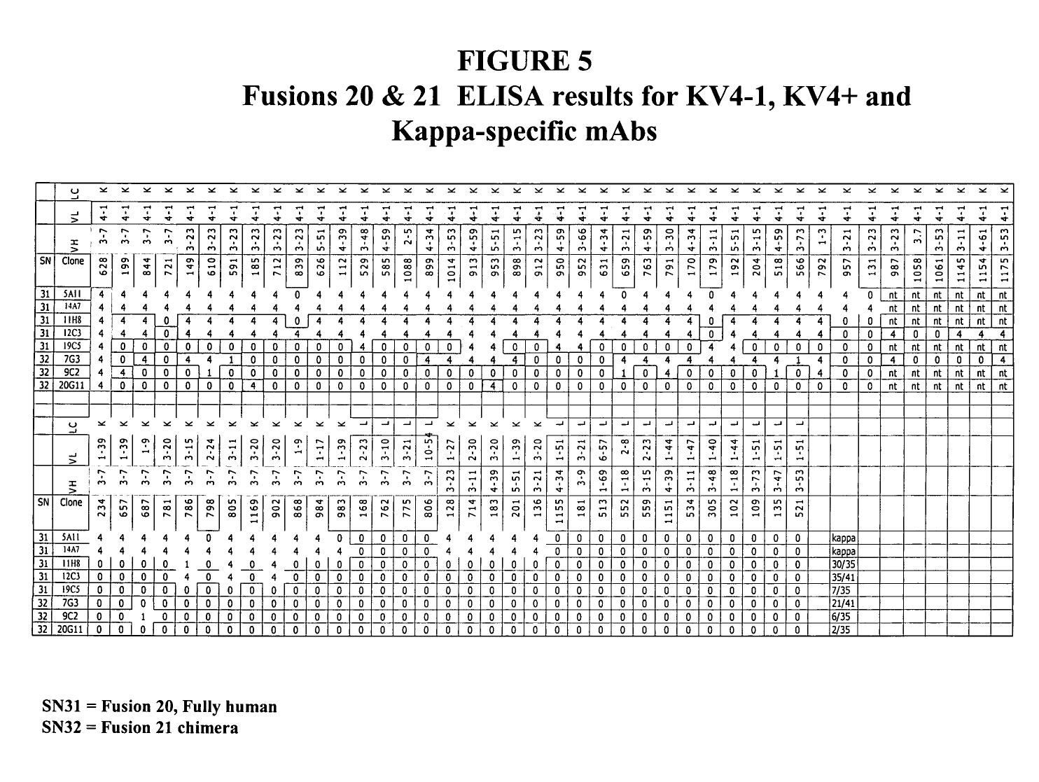

Fusion 21 is from animals immunized with chimeric Id protein using PIN628 V regions. Clone 7G3, a KV4-1-specific mAb generated from PIN628 chimeric Id protein recognizes 51% (21 of 41) of the KV4-1 Id proteins screened and although this mAb has a different pattern of recognition than clones 15E1 and 10H7 from fusions 13 and 14 respectively, the percent of KV4-1 coverage is similar, 67% (see FIG. 5). There are two clones that recognize most of the KV4-1 Id proteins and three of the KV3 Id proteins tested (clone 11H8 and clone 12C3). FIG. 5 shows the following KV4-1 specific clones: 19C5, 9C2, 20G11, 11H8, 12C3, and 7G3. No mAb were generated to HV3-48.

Study Number 33

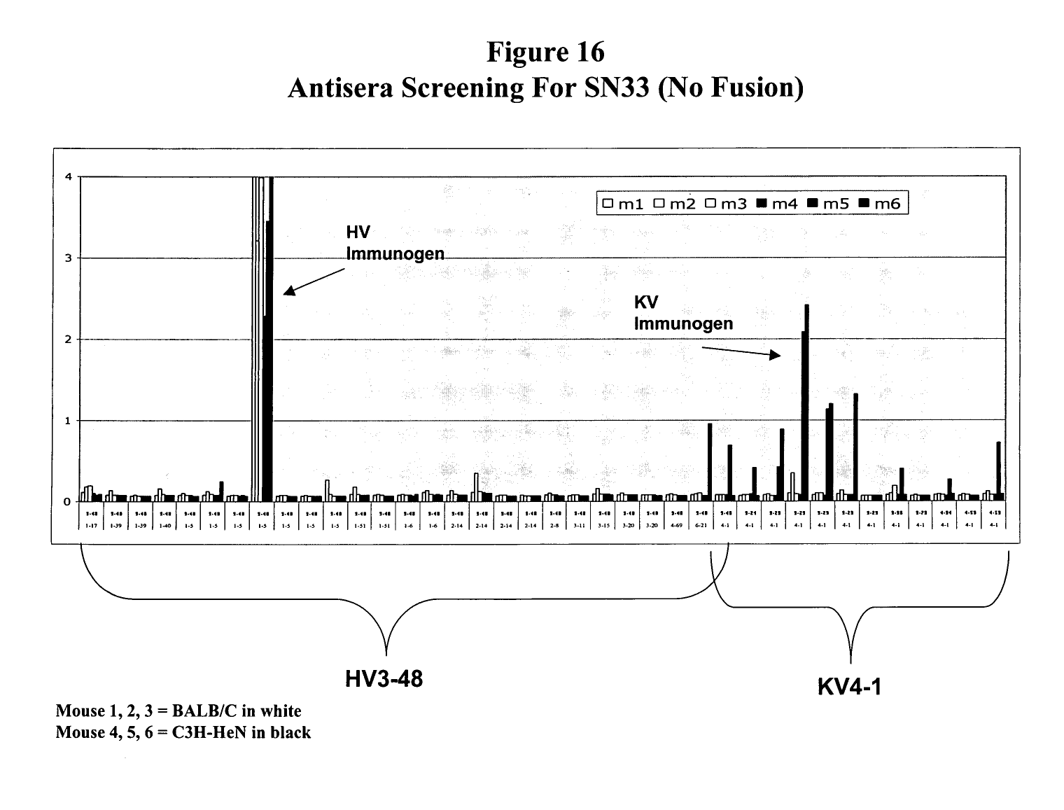

There was no fusion performed in this study. To test the influence of the HV region on the B cell response of the light chain V region (KV in this example) two different chimera Id proteins were generated using the same KV region. PIN149/149 chimera and PIN607/149 chimera Id proteins were constructed using the same light chain, KV4-1 from PIN149. Antisera from BALB/C mice immunized with PIN149/149 chimera Id protein and hybridomas generated from these mice demonstrated a B cell response to both the heavy chain and light chain. Hybridomas from fusions 13 and 14 resulted in HV3-23-and KV4-1-specific mAb. In contrast, BALB/C mice immunized with PIN607/149 chimera Id protein only responded to PIN607 (see FIGS. 15 and 16 antisera screening for fusions 13 and 14 and SN33). It was concluded from this experiment that the pairing of HV and (or HV/LV) likely influences the B cell repertoire and therefore the outcome of hybridoma specificity.

Fusion 22

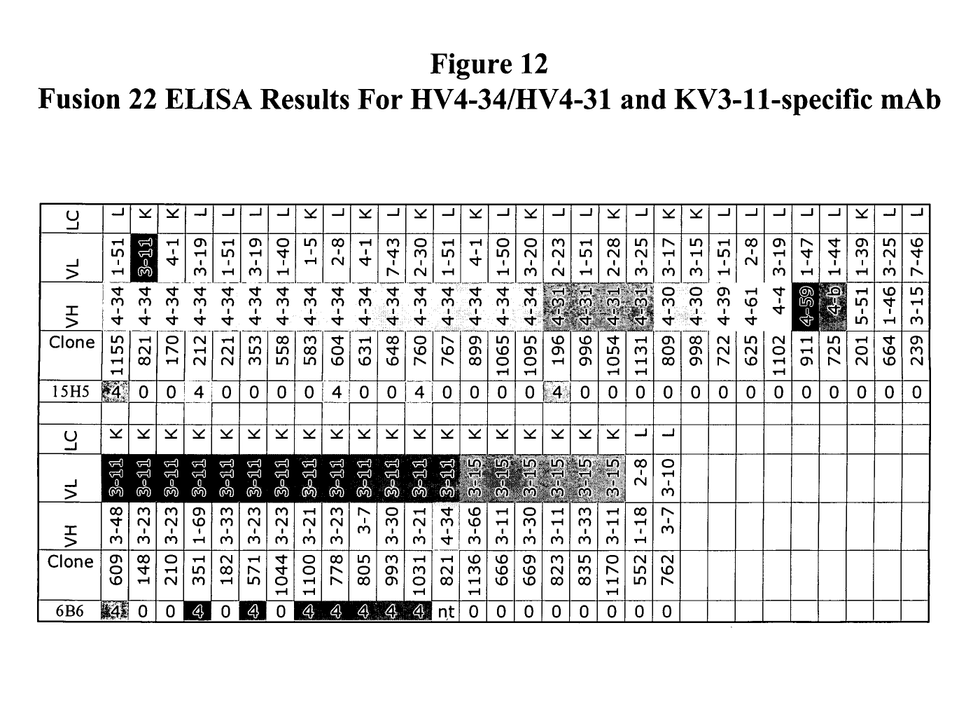

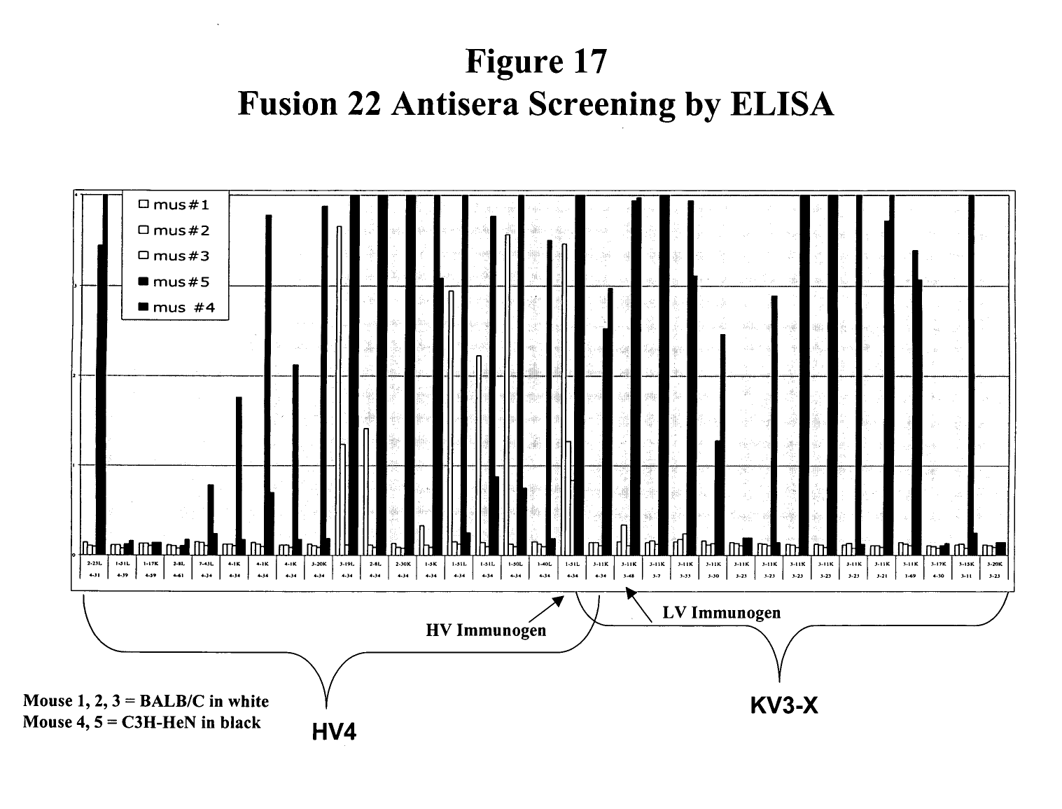

A difference in immune response between two mouse stains is demonstrated in this study. The antisera screening from animals immunized with PIN1155 (HV4-34)/PIN609 (KV3-11) chimera Id protein suggested that one could potentially obtain anti-HV4 and anti-KV3 mAbs from C3H-HeN but only anti-HV4-34 mAbs from BALB/C mice (see FIG. 17). The fusion was done with the one responsive HV4-34 reactive BALB/C mouse#1 and C3H-HeN mouse #4 and #5. After identifying two anti-Id clones that recognize the heavy chain, this left one HV4 family-specific mAb, 15H5, that recognize a subset of HV4 family members tested, 3 of 15 HV4-34 and 1 of 4 HV4-31. One clone, 6B6, was isolated that recognizes a subset of KV3-11 Id proteins tested (7 of 11) (see FIG. 12). All of the hybridomas were derived from C3H-HeN mice.

Fusion 23

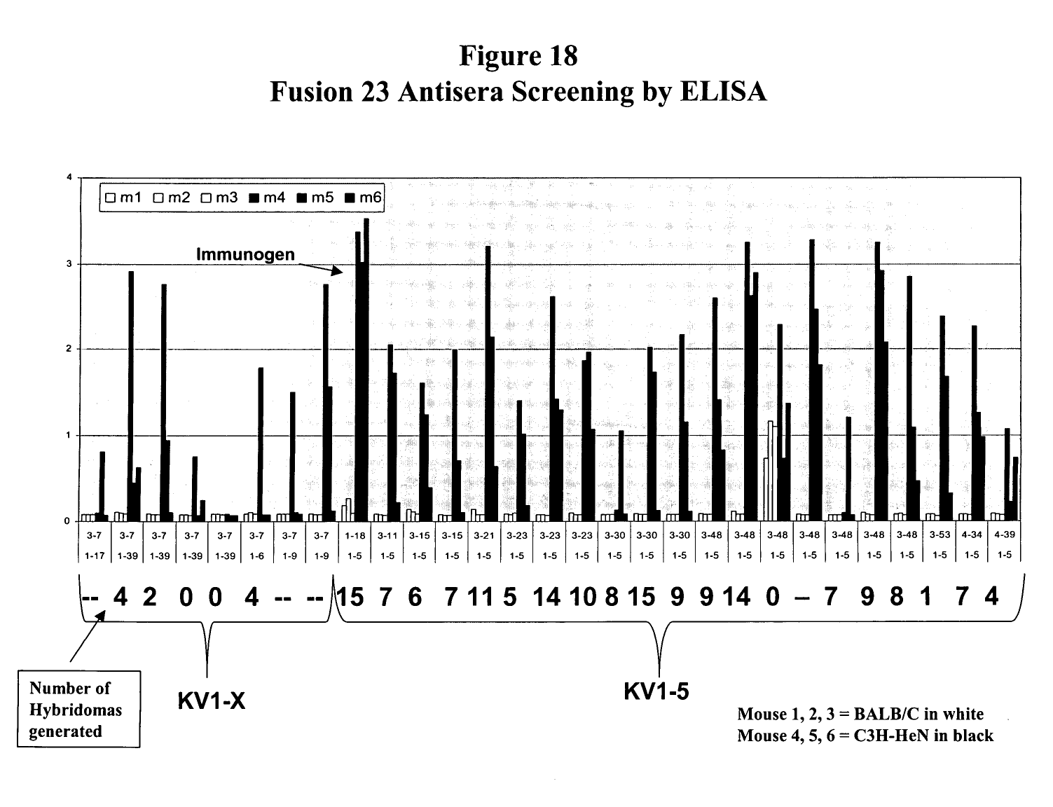

The antisera screen from mice immunized with PIN655/1092 chimera Id protein was very useful for predicting fusion outcome (see FIG. 18). The screen strongly suggested that C3H-HeN mice were very reactive against KV1-5 and other KV1 family members but there was no response to the heavy chain HV3-7, not even against the immunogen heavy chain PIN655 fully human Id protein. BALB/C mice did not respond to either HV3-7 or KV1-5 expressing Id proteins tested. A fusion with the three C3H-HeN mice resulted in 284 positive parental wells (from a total of 3000 wells screened) reactive with the fully human PIN1092 (KV1-5) Id protein. A second screen with the 284 hybridoma supernatants screened positive against the immunogen were tested against six KV1-5 expressing Id proteins, the immunogen light chain (PIN1092) Id proteins, and one non-family member related Id protein.

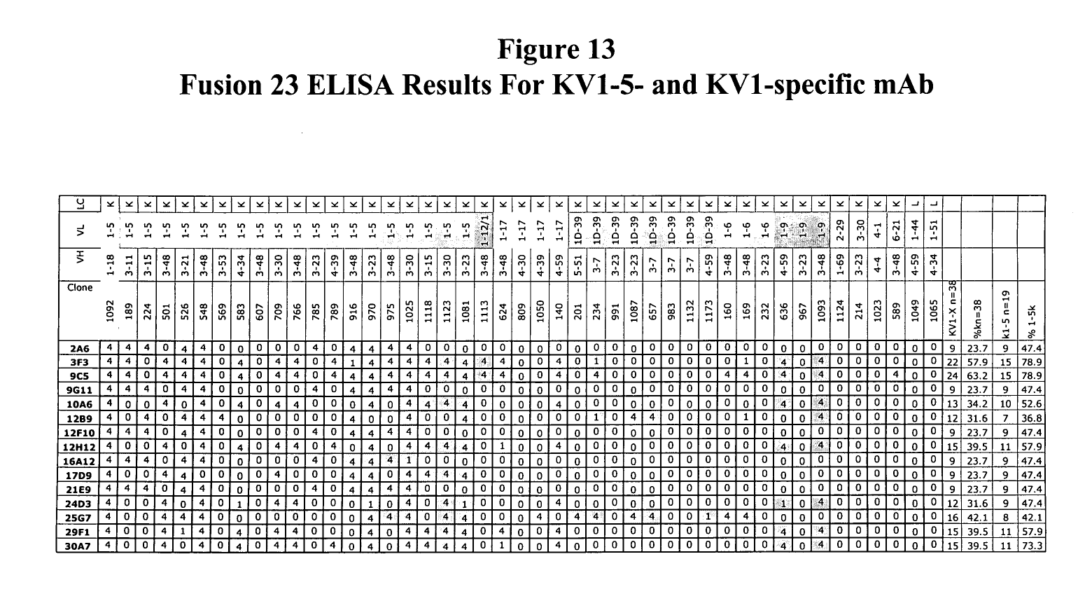

This screen resulted in 177 clones reactive against the immunogen (absorbency signal of 1.0 OD or higher). Of these, 15 hybridomas were immunoreactive to 4-6 of the KV1-5 Id proteins and were further characterized (FIG. 13). Six of the 15 clones (2A6, 9G11, 12F10, 16A12, 17D9, and 21E9) are KV1-5-specific, testing positive 9 of 15 KV1-5 Id proteins). Eight of 15 clones are KV1 reactive (3F3, 10A6, 12B9, 12H12, 24D3, 25G7, 29F1, and 30A7) and one clone is KV1+KV6-21 reactive (9C5) (see FIG. 13)).

Beginning with this fusion, a few minor changes were incorporated into the fusion protocol. The changes were directed at increasing the fusion efficiency (i.e. increasing the hybridoma numbers) basically by reducing cell toxicity caused by unnecessary exposure to chemicals, pH changes, and using protein-free media. The amount of time cells were exposed to PEG was also reduced. Gentimicin was eliminated as an antibiotic in the media. Selection of HGPRT positive clones was done using azaserine, replacing aminopterin. Azaserine was added to media just prior to fusion and not used in subsequent feedings. Parent hybridomas were fed on day 5 post fusion only and thereafter only as needed. This represents a reduction in the number of times the cells were manipulated. The new fusion protocol also includes maintaining cells in RPMI-1640 media except while cells are being fused with PEG. An increase in fusion efficiency was observed.

Fusion 24 and 25

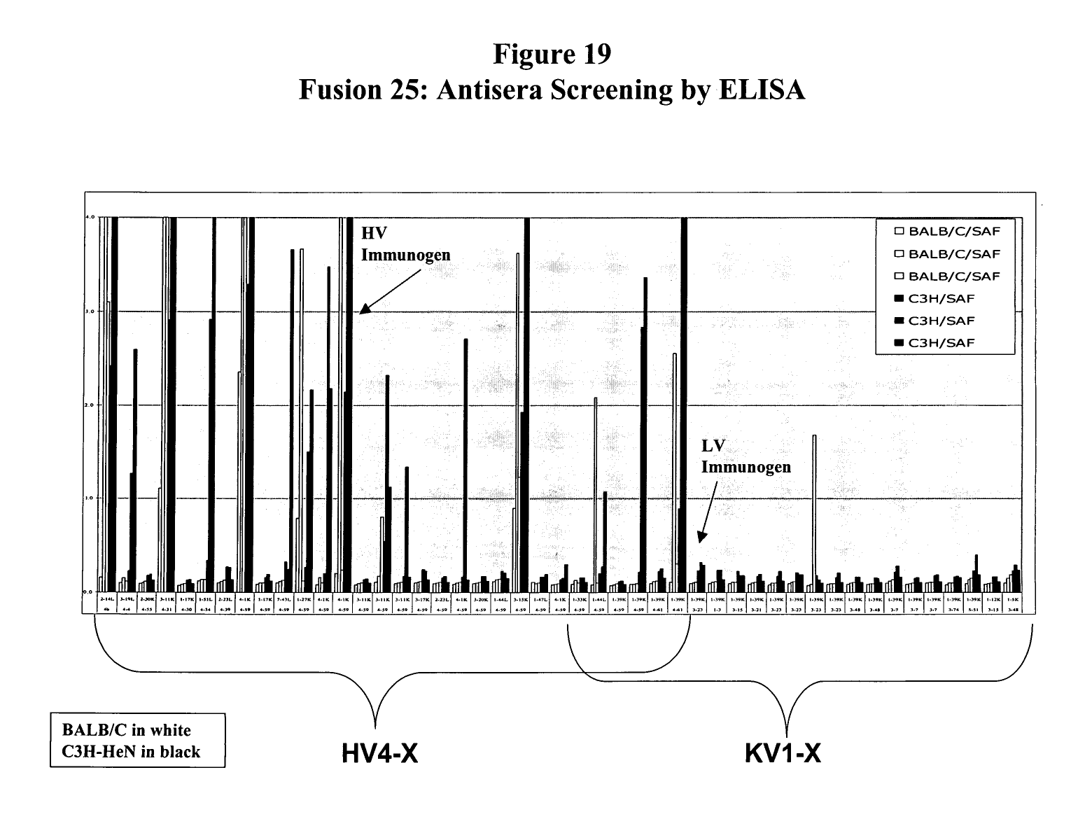

The antisera screening for SN37 is a good example demonstrating the dominant immune response of some V regions. C3H-HeN mice, and to a lesser degree BALB/C mice, responded to the heavy chain immunogen PIN913 (HV4-59) but not to the light chain PIN1062 (KV1-39). In addition the C3H-HeN response suggests a potentially broad reactivity to HV4 family members (see FIG. 19). In addition to responding to several, 10 of 20, HV4-59 expressing Id proteins the response extends to other HV4 family members including HV4-31, 4-34, 4-61, 4-4, 4b, but not against the one or two 4-30, 4-39, 4-55 expressing Id proteins.

### EXAMPLE 3

### Further Characterization of Selected Clones

This example describes further characterization of certain clones described in Example 2. The variable regions from the following six clones were sequenced using standard sequencing procedures: clone 3C9, which is specific for family member HV3-23; clone 10H7, which is specific for family member KV4-1; clone 12C3, which is specific for family member KV4-1; clone 20H5, which is specific for family LV1; clone 15E8, which is specific for family LV2; and clone 4H11, which is cross reactive with VL2 and LV3-25. Binding constants were also determined for three of the clones that were sequences (clones 3C9, 10H7, and 20H5), as well as for two additional clones (clone 6C9, which is specific for HV-23, and clone 15E1, which is specific for KV4-1).

FIG. 6 shows the results of sequencing clone 3C9, which is specific for family member HV3-23. In particular, FIG. 6A shows the amino acid sequence (SEQ ID NO:11) and nucleic acid sequence (SEQ ID NO:12) of the heavy chain variable region for this clone, while FIG. 6B shows the amino acid sequence (SEQ ID NO:13) and nucleic acid sequence (SEQ ID NO:14) of the light chain variable region for this clone. The three CDRs in each sequence are underlined in each sequence.

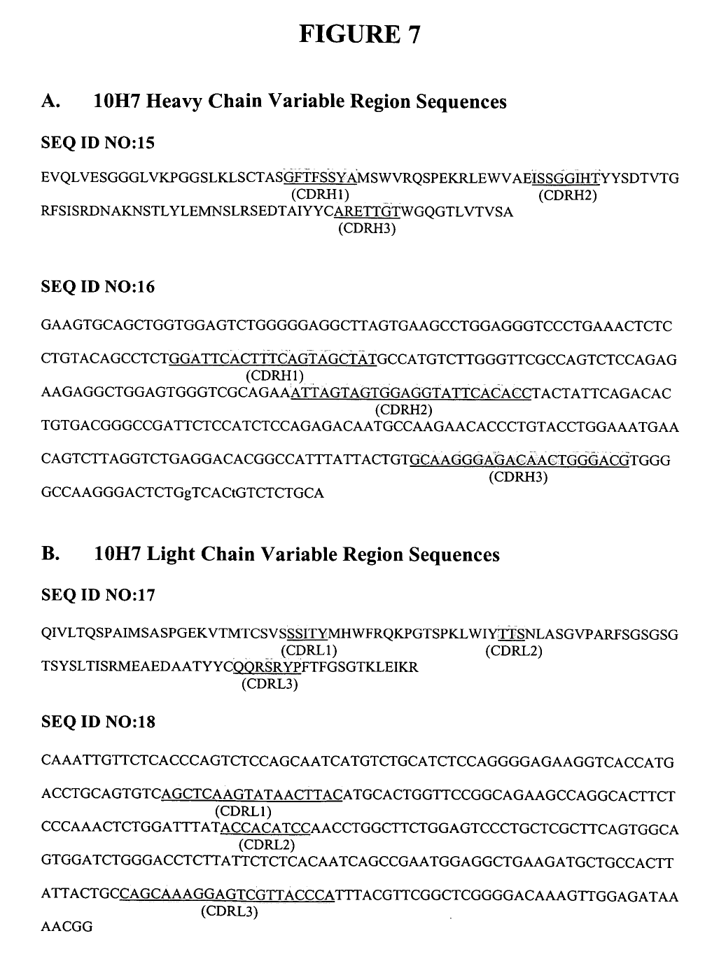

FIG. 7 shows the results of sequencing clone 10H7, which is specific for family member KV4-1. In particular, FIG. 7A shows the amino acid sequence (SEQ ID NO:15) and nucleic acid sequence (SEQ ID NO:16) of the heavy chain variable region for this clone, while FIG. 7B shows the amino acid sequence (SEQ ID NO:17) and nucleic acid sequence (SEQ ID NO:18) of the light chain variable region for this clone. The three CDRs in each sequence are underlined in each sequence.

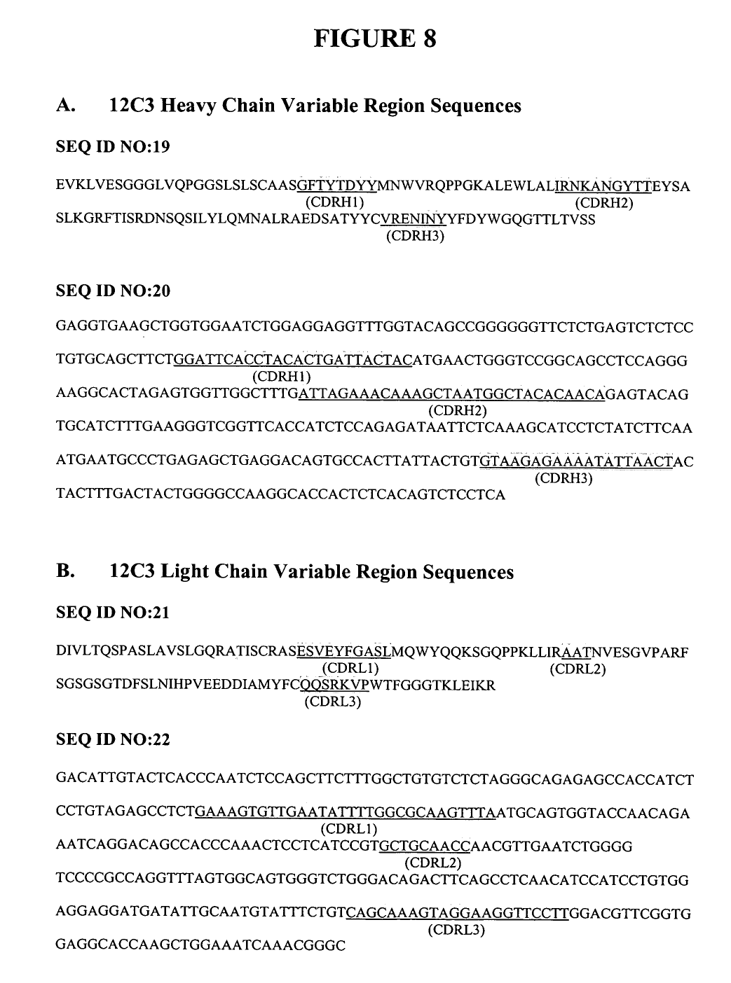

FIG. 8 shows the results of sequencing clone 12C3, which is specific for family member KV4-1. In particular, FIG. 8A shows the amino acid sequence (SEQ ID NO:19) and nucleic acid sequence (SEQ ID NO:20) of the heavy chain variable region for this clone, while FIG. 8B shows the amino acid sequence (SEQ ID NO:21) and nucleic acid sequence (SEQ ID NO:22) of the light chain variable region for this clone. The three CDRs in each sequence are underlined in each sequence.

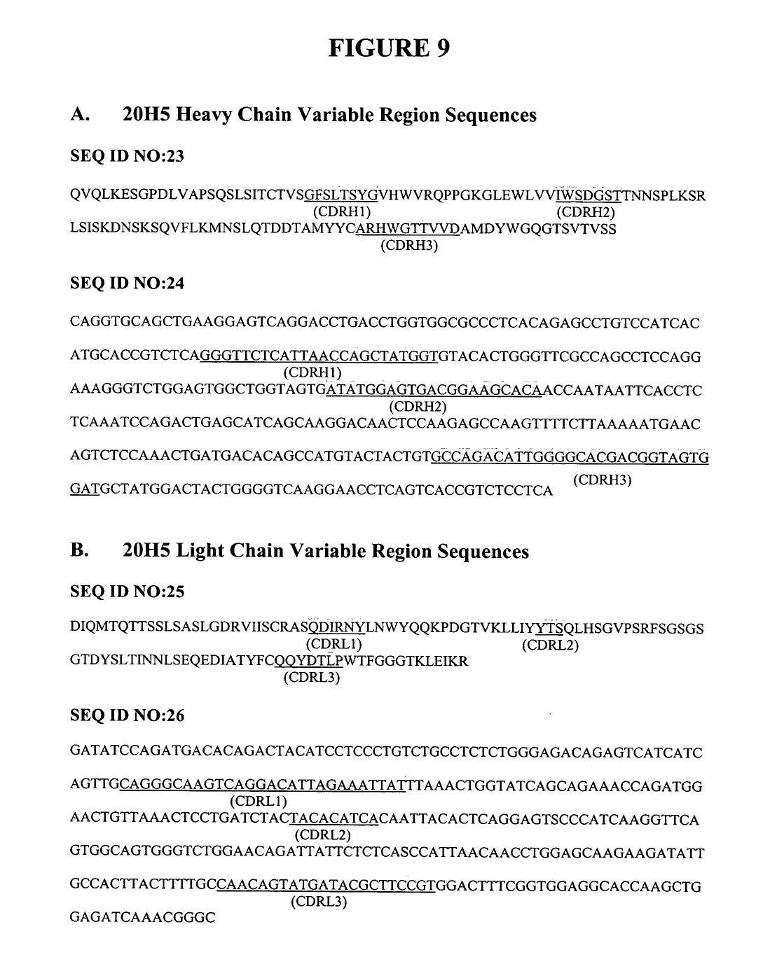

FIG. 9 shows the results of sequencing clone 20H5, which is specific for family LV1. In particular, FIG. 9A shows the amino acid sequence (SEQ ID NO:23) and nucleic acid sequence (SEQ ID NO:24) of the heavy chain variable region for this clone, while FIG. 9B shows the amino acid sequence (SEQ ID NO:25) and nucleic acid sequence (SEQ ID NO:26) of the light chain variable region for this clone. The three CDRs in each sequence are underlined in each sequence.

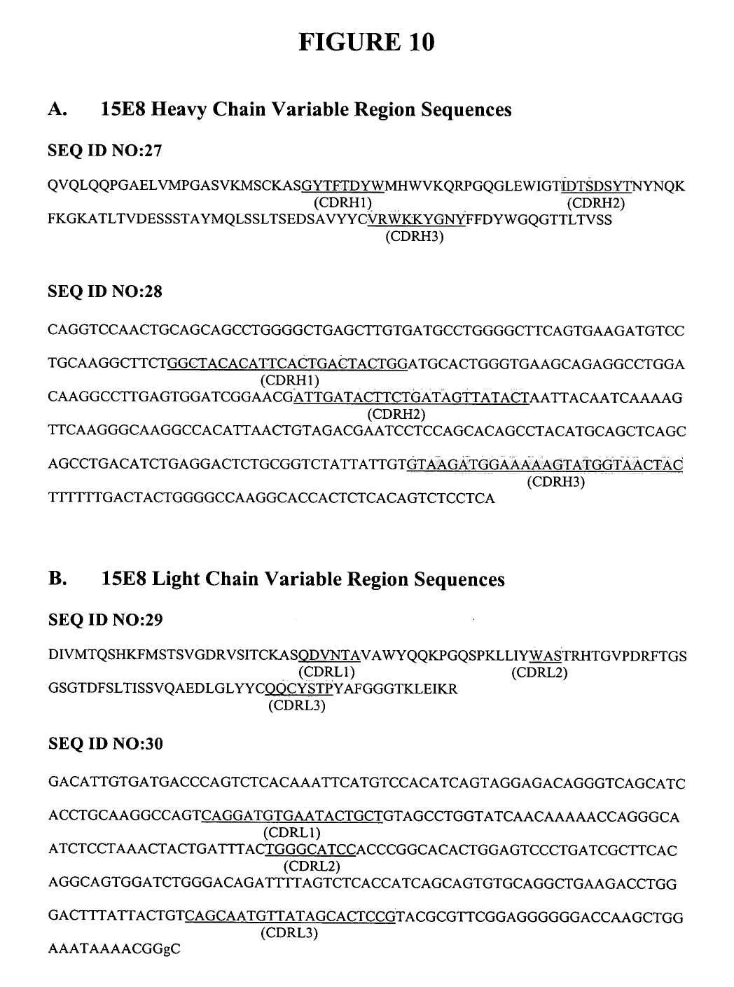

FIG. 10 shows the results of sequencing clone 15E8, which is specific for family LV2. In particular, FIG. 10A shows the amino acid sequence (SEQ ID NO:27) and nucleic acid sequence (SEQ ID NO:28) of the heavy chain variable region for this clone, while FIG. 10B shows the amino acid sequence (SEQ ID NO:29) and nucleic acid sequence (SEQ ID NO:30) of the light chain variable region for this clone. The three CDRs in each sequence are underlined in each sequence.

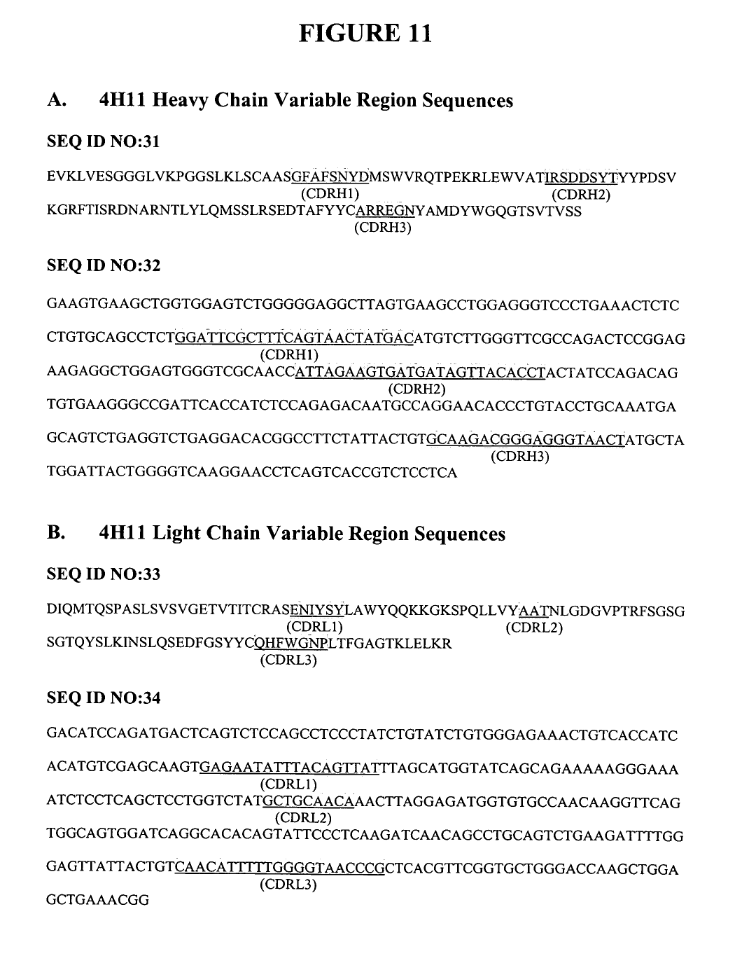

FIG. 11 shows the results of sequencing clone 4H11, which is cross reactive with VL2 and LV3-25. In particular, FIG. 11A shows the amino acid sequence (SEQ ID NO:31) and nucleic acid sequence (SEQ ID NO:32) of the heavy chain variable region for this clone, while FIG. 11B shows the amino acid sequence (SEQ ID NO:33) and nucleic acid sequence (SEQ ID NO:34) of the light chain variable region for this clone. The three CDRs in each sequence are underlined in each sequence.

Binding constants were also determined for three of the sequenced clones (3C9, 10H7, and 20H5), as well as for two additional clones (6C9 and 15E1). The association and disassociation rates were measured by surface plasmon resonance using a BIACORE 2000 machine. These results are presented in Table 4 below.

TABLE 4

| mAb | Idiotype | | | | | | |
|---|---|---|---|---|---|---|---|
| Mouse | Human | ka (M-1s-1) | kd (s-1) | KD (nM) | ka (M-1s-1) | kd (s-1) | KD (nM) |
| 3C9 | Immunogen (a) | 7.49(1)e4 | 1.34(3)e−4 | 1.78(4) | | | |
| 6C9 | Immunogen | 7.18(4)e4 | 9.6(3)e−5 | 1.34(4) | 6.8(2)E3 | 3.64(8)E−3 | 530(20) |
| 10H7 | Immunogen | 4.54(1)e4 | 3.8(3)e−5 | 0.83(7) | | | |
| 15E1 | Immunogen | 1.08(2)e5 | 7.6(1)e−5 | 0.71(9) | 2.30(4)e4 | 3.16(2)E−3 | 137(2) |
| 3C9 | Non-binder (b) | no binding | | | | | |
| | | detected | | | | | |
| 6C9 | Non-binder | no binding | | | | | |
| | | detected | | | | | |
| 10H7 | Binder (c) | 3.317(4)e4 | 9.9(2)e−5 | 3.00(7) | | | |
| 15E1 | Binder | 4.44(1)e4 | 1.423(4)e−7 | 3.28(1) | 1.01(1)e4 | 9.31(3)e−3 | 917(6) |
| 20H5 | Immunogen | 7.59(2)e4 | 1.46(5)e−4 | 1.92(7) | | | |
| 20H5 | Non-binder | no binding | | | | | |
| | | detected | | | | | |

(a) Immunogen = Id protein used to generated the mAb
(b) Non-binder = mAb did not bind Id protein in ELISA
(c) Binder = mAb bound Id protein in ELISA

In addition, based on ELISA results, PIN185 was used as a positive control Id protein for 10H7 and 15E1 and a negative control Id protein for 3C9 and 6C9 and PIN149 was used as a non-binding Id protein for 20H5. Each mAb was captured onto an anti-mouse surface and tested for binding to the fully human Id protein. Following the mAb capture phase of the assay for the (@2 ug/ml), the fully human Id proteins were all diluted to a starting concentration of 250 nM and tested in a three fold dilution series. Each concentration was tested in duplicate. The running buffer contained PBS plus 0.005% tween-20 and 0.1 mg/ml BSA as a carrier. Binding data were collected at 25° C. The mouse mAbs 2 and 4 displayed complex binding kinetics. These data were fit with a two independent site model. The other data sets all fit well to a single site interaction model.

### EXAMPLE 4

### Generating Additional mAbs

This example describes methods that could be used to generate additional anti-LV 1, anti-LV2, anti-LV2-8, anti-KV4-1, anti-HV3-23, and anti-LV2/LV3-35 monoclonal antibodies. In order to generate additional mAbs, one could use, for example, the methods described in Example 2 above. One could employ similar expression constructs as described in Example 2 using the same or different variable regions. For example, one could use the same variable regions as described in Example 2 as part of the immunogen (see FIG. 1), one could employ variants of these sequences (e.g. sequences shown in FIG. 1 with the framework 1 regions altered by one or two amino acids), one could employ germline variable regions, or a variant of a germline variable region (e.g. a known germline sequence with the framework 1 region altered by one or two amino acids).

In order to generate additional anti-HV3-23 mAbs, for example, one could use an expression construct that expresses the HV3-23 heavy chain variable region shown in SEQ ID NO:3 (see FIG. 1), or one of the three known HV3-23 germline alleles, which may be found under Genebank accession numbers (nucleic acid): M99660, M35415, and U29481. Framework 1 region variants of these variable regions (e.g. altered by a limited number of amino acid substitutions) may also be employed. In certain embodiments, germline HV3-23 variable regions are preferred as mAbs generated therefrom may recognize a large percent of HV3-23 type idiotypic proteins (e.g. idiotypic proteins derived from the tumors of NHL patients).

In order to generate additional KV4-1 mAbs, for example, one could use an expression construct that expresses the KV4-1 kappa chain variable regions shown in SEQ ID NO:4 or SEQ ID NO:10 (see FIG. 1), or the KV4-1 germline sequence that is known, which is found under Genebank accession number (nucleic acid) Z00023. Framework 1 region variants of these variable regions (e.g. altered by a limited number of amino acid substitutions) may also be employed. In some embodiments, germline KV4-1 variable regions are preferred as mabs generated therefrom may recognize a large percent of KV4-1 idiotypic proteins (e.g. idiotypic proteins derived from the tumors of NHL patients).

In order to generate additional LV1 specific mAbs, or LV1 family member specific mAbs, one could use, for example, an expression construct that expresses an LV1 variable region, such as the LV1-40 variable region shown in SEQ ID NO:2 (see FIG. 1), or one of the following LV1 germline sequences which are found in Genebank (nucleic acid): A) germline LV1-40 accession numbers M94116, X53936, and Z22192, B) germline LV1-41 accession numbers: M94118 and D87010, C) germline LV1-44 accession number Z73654, D) germline LV1-47 accession numbers Z73663, and D87016; E) germline LV1-50 accession number M94112, and F) germline LV1-51 accession numbers Z73661 and M30446. Framework 1 region variants of these variable regions (e.g. altered by a limited number of amino acid substitutions) may also be employed. In particular embodiments, germline LV1 variable regions (e.g. LV1-4) are preferred as mAbs generated therefrom may recognize a large percent of LV1 idiotypic proteins (e.g. idiotypic proteins derived from the tumors of NHL patients).

In order to generate additional LV2 specific mAbs, or LV2 family member specific mAbs, one could use, for example, an expression construct that expresses an LV2 variable region, such as the LV2-8 variable region shown in SEQ ID NO:6 (see FIG. 1), or one of the following LV2 germline sequences which are found in Genebank (nucleic acid): A) germline LV2-11, accession numbers Z73657, Z22198, and Y12415, B) germline LV2-14 accession numbers Z73664, L27822, Y12412, and Y12413, C) germline LV2-18 accession numbers Z73642, L27697, L27694, and L27692, D) germline LV2-23 accession numbers X14616, Z73665, and D86994, E) germline LV2-33 accession numbers Z73643, L27823, and L27691, and F) germline LV2-8 accession numbers X97462, L27695, and Y12418. Framework 1 region variants of these variable regions (e.g. altered by a limited number of amino acid substitutions) may also be employed. In particular embodiments, germline LV2 variable regions (e.g. LV2-8) are preferred as mAbs generated therefrom may recognize a large percent of LV2 idiotypic proteins (e.g. idiotypic proteins derived from the tumors of NHL patients).

### EXAMPLE 5

### Chimeric Antibody Construction

This Example describes the construction of chimeric monoclonal antibodies. This procedure could be used, for example, to generate chimeric antibodies of the mAbs discussed in the previous Examples.

Identification of Monoclonal Ab Variable Region Sequences

Total RNA is purified from frozen hybridoma cells (aprox. 10⁷ cells) using a QIAshredder column (Qiagen GmbH, Hilden, Germany) followed by an RNeasy mini kit (Qiagen GmbH, Hilden, Germany) according to the manufacturer's instructions, and serves as a template for first strand cDNA synthesis. Reverse transcription is primed using four primers (in 4 separate reactions) that hybridize to sequences within the mouse immunoglobulin (Ig) constant (C) region genes: mmG2 (5′ AGGGAAATAACCTTTGACC AGGCAT3′, SEQ ID NO:51); mmG3 (5′ CTAGACAGGGATCCAGAGTTCCA3′, SEQ ID NO:52); mnK2 (5′ ACGACTGAGGCACCTCCAGATGTT3′, SEQ ID NO:53); and mmK3 (5′ TGGGGTAGAAGTTGTTCAAGAA 3′, SEQ ID NO:54) and performed with rTth DNA polymerase (Applied Biosystems, Foster City, Calif.) in the presence of manganese acetate according to manufacturer's instructions. The resulting cDNA are further purified using a QIAquick PCR purification kit (Qiagen GmbH, Hilden, Germany) according to manufacturer's instructions.

Using the purified first stand cDNA as template, anchor PCR is carried out to identify which V regions are utilized for expression of the immunoglobulin heavy and light chains in the hybridoma sample. The procedure involves dGTP tailing of the 1st strand cDNA with terminal transferase (TdT)(Roche Applied Sciences, Indianapolis, Ind.) in the presence of cobalt chloride according to manufacturer's instructions with the exception of using the 5×rTdT Buffer supplied by USB Corp. (Cleveland, Ohio) in place of the supplied Roche 5× reaction buffer. The polyG tailed cDNA is then purified using a QIAquick PCR purification kit (Qiagen GmbH, Hilden, Germany) according to manufacturer's instructions.

Purified polyG tailed cDNA is then PCR amplified with primer An8cvH, AnlOcvH (5′TCTAGAATTCACGCGTCCCCCCCCCC 3′, SEQ ID NO:55) and Anl2cvH (5′TCTAGAATTCACGCGTCCCCCCCCCCCC 3′, SEQ ID NO:56), in separate reactions, as the forward primers and the appropriate constant primer (mmGl [5′CAGGGGCCAGTGGATAGAC 3′, SEQ ID NO:57], mmG2 [5′GATGGTGGGAAGA TGGATACAGTT 3′, SEQ ID NO:58], mmK1 or mmK2) as a reverse primer. PCR amplifications are performed with Pfu DNA polymerase (Stratagene, San Diego, Calif.) according to the manufacturer's instructions for 30 cycles using the following profile: 94° C. for 40 seconds, 63° C. for 40 seconds, and 72° C. for 80 seconds.

Amplification products are then electrophoresed on a 1.8% agarose TAE gel and excised for further purification. The An8cvH, An10cvH, and An12cvH gel bands are excised as one band for each of the four constant chains (mmG1, mmG2, mmK1 and mmK2), resulting in four excisional amplification bands. The amplification gel bands are then purified using a QIAquick Gel Extraction kit (Qiagen GmbH, Hilden, Germany) according to the manufacturer's instructions. Each product is then ligated into vectors and transformed into E. coli using a Zero Blunt TOPO PCR Cloning Kit For Sequencing with One Shot TOPIO Chemically Competent E. coli (Invitrogen, Carlsbad, Calif.) according to the manufacturer's instructions. Each transformation is then plated onto two LB agar+100 μg ml carbenicillin plates and incubated overnight at 37° C.

These transformation colonies are then PCR screened using M13 Forward (5′ GTAAAACGACGGCCAG 3′, SEQ ID NO:59) and M13 Reverse (5′ CAGGAAACAGCTATGAC 3′, SEQ ID NO:60) primers and AmpliTaq DNA polymerase (Applied Biosystems, Foster City, Calif.) according to the manufacturer's instructions using the following profile: 1 cycle at 94° C. for 5 minute, 30 cycles of the following: 94° C. for 20 seconds, 53° C. for 20 seconds, and 72° C. for 80 seconds.

The products from the screening reaction are then electrophoresed on a 2.2% agarose TAE gel. The PCR screening products then serve as template for DNA sequencing. DNA sequencing is performed with 1 μl of PCR product and M13 forward and M13 reverse sequencing primers using Big Dye Terminator v3.1 Cycle Sequencing kit (Applied Biosystems, Foster City, Calif.) according to manufacturer's instructions using the following thermal cycling profile: initial denaturation cycle of 96° C. for 1 minute followed by 25 cycles of: 96° C. for 10 seconds, 50° C. for 5 seconds, and 60° C. for 60 seconds.

Cycle sequencing reactions are then subjected to ethanol precipitation in the presence of sodium acetate, dried, and resuspended in 20 μl of Hi-Di Formamide (Applied Biosystems, Foster City, Calif.). Reactions are then denatured at 95° C. for 5 minutes and loaded onto an ABI Prism 3100 Genetic Analyzer (Applied Biosystems, Foster City, Calif.) and subjected to capillary electrophoresis according to manufacturer's instructions. Sequence data is visualized using the Lasergene Software Suite (DNASTAR, Inc., Madison Wis.).

Preparation of Variable Regions for Cloning

Two primers are used which are designed to make PCR product for cloning of the heavy chain. The forward primer (4H11H_F, 5′ TCTAGAATTCACGCGTCCACCATGAACTTTGGGCTGA 3′, SEQ ID NO:61) is designed 5′ of framework 1 complimentary to the first sixteen nucleotides starting at the initiating ATG and includes twenty-one nucleotides for Xba I, EcoR I and Mlu I restriction sites. The reverse primer (4H11H_JH, 5′ GAGGGGCCCTTGGTCGACGCTGAGGAGACGGTGACTGA 3′, SEQ ID NO:62) is designed for reverse complimentary to the last eighteen nucleotides of JH and first twenty nucleotides of the human gamma constant containing Apa I and Sal I restriction sites. PCR amplification is performed using rTth DNA polymerase XL (Applied Biosystems, Foster City, Calif.) according to the manufacturer's instructions for 30 cycles using the following profile: 94° C. for 20 seconds, 63° C. for 20 seconds, and 72° C. for 80 seconds. amplification product is then electrophoresed on a 1.8% agarose TAE gel, excised and purified using a QIAquick Gel Extraction kit (Qiagen GmbH, Hilden, Germany) according to the manufacturer's instructions.

Cloning the light chain employes extension by overlap PCR. For the variable region, the forward primer (4H11K_F, 5′ TCTAGAATTCACGCGTCCACCATGAGTGTGCCCACTCA 3′, SEQ ID NO:63) is designed 5′ of framework 1 complimentary to the first fifteen nucleotides starting at the intiating ATG and includes twenty-one nucleotides for Xba I, EcoR I and Mlu I restriction sites. The reverse primer (4H11K_JKR, 5′ TGCAGCCACAGTCCGTTTCAGCTCCAGCTTGGTCCC 3′, SEQ ID NO:64) is designed for reverse complimentary to the last twenty-four nucleotides of the mouse J region and first twelve nucleotides of the human constant. For the constant region, the forward primer (4H11K_JKF, 5′ GAGCTGAAACGGACTGTGGCTGCACCTTCTGTCTTC 3′, SEQ ID NO:65) is designed complimentary to the last twelve nucleotides of the mouse J region and twenty-four nucleotides of the human kappa constant region. The reverse primer for use during the amplification is the thirty-four bp CK primer (SEQ ID NO:49) which is the reverse compliment to the human kappa constant starting twenty-nine nucleotides 3′ of the end of JK, which contain Afl II and BspE I restriction sites.

The overlap extension PCR primers discussed above (4H11K_JKR and 4H11K_JKF) are designed to anneal to each other's sequence during the overlap extension PCR. The first twenty-four nucleotides of 4H11K_JKR are complimentary to the last twenty-four nucleotides of 4H11K_JKF. To synthesize PCR product for light chain cloning three separate PCR reactions are performed. Initially PCR product of the mouse light chain variable region is made using 4H11K_F and 4H11K_JKR primers using hybridoma cDNA as template. The second PCR product made is the human constant using 4H11K_JKF and CK primers using pSRαSD79CKWT vector as template. These first two reactions are performed using rTth DNA polymerase XL (Applied Biosystems, Foster City, Calif.) according to the manufacturer's instructions for 30 cycles using the following profile: 94° C. for 20 seconds, 63° C. for 20 seconds, and 72° C. for 80 seconds. The amplification products (4H11K_F/4H11K_JKR, 4H11K_JKF/CK) are then electrophoresed on a 1.8% agarose TAE gel, excised and purified separately using a QIAquick Gel Extraction kit (Qiagen GmbH, Hilden, Germany) according to the manufacturer's instructions.

Overlap extension PCR is carried out by using the product from the first and second light chain reactions as template. 4H11K_F and CK primers are added to this reaction and performed using Pfu DNA polymerase (Stratagene, San Diego, Calif.) according to the manufacturer's instructions for 20 cycles using the following profile: 94° C. for 40 seconds, 63° C. for 40 seconds, and 72° C. for 80 seconds. The amplification product (4H11K _F/CK) is then electrophoresed on a 1.8% agarose TAE gel, excised and purified using a QlAquick Gel Extraction kit (Qiagen GmbH, Hilden, Germany) according to the manufacturer's instructions.

Cloning of Variable Regions onto Human Constant Regions

Heavy chain purified amplification product is then sub-cloned into pSRαSD79CG1WT expression vector and light chain purified amplification product is sub-cloned into pSRαSD79CKWT expression vector. For the heavy chain sub-cloning both the purified amplicon (4H11H_F/4H11H_JH) and pSRαSD79CG1WT are digested with EcoR I and Sal I. The light chain amplicon (4H11K_F/CK) and pSRαSD79CKWT are digested with EcoR I and Afl II. The digests are then electrophoresed on a 1.8% agarose TAE gel and excised for further purification. The digested heavy chain amplicon and pSRαSD79CG1WT vector gel bands are combined into a single tube. The digested light chain amplicon and pSRαSD79CKWT vector gel bands are combined into a single tube. These heavy and light chain products are then purified using a QlAquick Gel Extraction kit (Qiagen GmbH, Hilden, Germany) according to the manufacturer's instructions. Ligations of the purified amplicons with the appropriate vectors are performed using T4 DNA Ligase (New England Biolabs, Ipswich, Mass.) according to the manufacturer's instructions and transformed into E. coli using a DH5α Library Efficiency Competent Cells (Invitrogen, Carlsbad, Calif.) according to the manufacturer's instructions. Each transformation is then plated onto two LB agar+100 μg/ml carbenicillin plates and incubated overnight at 37° C.

The transformed colonies are then PCR screened using 5SD primer (5′ AGGCCTGTACGGAAGTGTTAC 3′, SEQ ID NO:66 ) and the appropriate CG or CK primers and AmpliTaq DNA polymerase (Applied Biosystems, Foster City, Calif.) according to the manufacturer's instructions using the following profile: 1 cycle at 94° C. for 5 minutes, 30 cycles of the following: 94° C. for 20 seconds, 53° C. for 20 seconds, and 72° C. for 80 seconds. The products from the screening reaction are then electrophoresed on a 2.2% agarose TAE gel. The screening products for colonies that are positive for inserts of the appropriate size are then sequenced using the 5SD and the appropriate CG or CK primers as described above to verify the clone is of the correct sequence.

Expression of Complete Chimeric Antibodies

DNA plasmid vectors containing the coding sequences for heavy and light chain mouse-human chimeric genes obtained as described above and the dhihydrofolate reductase (DHFR) gene are constructed as described above. The DNA mixture is electroporated into Chinese Hamster Ovary cells that are deficient in DHFR expression. After recovery, the cells are plated in growth medium that does not contain thymidine, glycine, or hypoxanthine for selection of cells that have incorporated the DHFR encoding vectors as well as the heavy and light chain DNA. Cells that survive the selection are expanded and then exposed to low levels of methotrexate in the medium, which is an inhibitor of DHFR and allows the selection of cells that have become resistant to the inhibitor by amplification of the integrated DHFR genes. Upon adequate expansion of the cells, cell supernatant is assayed for the concentration of secreted monoclonal antibody using an ELISA method for the detection of immunoglobulin. In brief, microtiter plates are coated with anti heavy chain specific antibodies. After blocking of the plate, diluted supernatant from the recombinant CHO cells is allowed to react with the coated plates. After washing away excess supernatant, bound recombinant antibodies are detected by first binding biotinylated anti light chain reactive antibodies followed by HRP-conjugated streptavidin. After washing, TMB substrate is added and allowed to develop. Clones of CHO cells demonstrating high production levels of monoclonal antibody are selected for additional rounds of growth in increasingly higher concentrations of methotrexate in order to bring about coordinate gene amplification that results in an increased specific productivity of the cells producing monoclonal antibody. For manufacturing purposes, the development of the CHO cell line also includes the adaptation of the cells for suspension growth in serum and animal protein-free media. Selection of the production cell line continues until a productivity target of at least 150 mg of protein per liter of cells is achieved.

Upon successful completion of cell line development, the cell line is re-cloned as necessary, tested for the presence of adventitious agents including virus, and further characterized for stability of protein production. Aliquots of the cells are frozen to serve as a Master Cell Bank. For a production run, an aliquot of the Master Cell Bank is thawed and the cells are expanded into increasingly larger growth vessels until a sufficient quantity of cells has been generated for inoculating a production bioreactor. Upon completion of the bioreactor culture, cell debris is separated from the crude harvest supernatant. Secreted monoclonal antibody is then captured by affinity chromatography on a Staphyloccocus aureus Protein A column for isolation of crude monoclonal product. The Protein A affinity-purified pool is then further purified on an ion exchange column. The final purified monoclonal is then sterile purified using a 0.2 micron filter. Material is diafiltered into the final formulation buffer and diluted in this buffer to a final concentration of 20 mg/ml. 20 ml (400 mg) aliquots are aseptically filled into sterile glass vials that are stoppered and crimp-sealed.

### EXAMPLE 6

### Directed Evolution Methods

As described above, mAb 3C9 is specific for family member VH3-23. This example describes the use of directed evolution type methods to identify additonal VH3-23 specific clones with optimized properties compared to the parental/donor 3C9 antibody using methods generally described in U.S. Pat. Pub. 20040162413 (herein incorporated by reference). Briefly, a library of light and heavy chain variable regions may be generated (e.g. as described in Example 2) that have nucleic acid sequences the same as 3C9 (see FIG. 6A and 6B, which provides SEQ ID NO:12 and SEQ ID NO:14) except for changes to CDR encoding regions. In particular, each amino acid position in some or all of the CDRa are individually randomized to include all amino acids except the 3C9 sequences shown in FIG. 6. It is noted that alternate frameworks, rather than the ones shown in FIG. 6 for 3C9, could be employed instead (e.g. human germline frameworks). This process generates libraries with a large diversity of variable region sequences. The DNA sequences are then annealed to uridinylated single stranded phage DNA such that the VL region is inserted between an appropriate signal sequence and a human CL region sequence. Similarly, the heavy chain fragment is designed to insert, in frame, between a signal sequence and the human CH1 region. The phage DNA and the DNA fragments are then mixed, heated to 75° C. and cooled to 20° C. over the course of 45 minutes. Double stranded DNA is then generated by the addition of T4 DNA polymerase and T4 DNA ligase with an incubation of 5 minutes at 4° C. followed by 90 minutes at 37° C. The reaction is then phenol extracted and the double stranded DNA precipitated by the addition of ethanol. The DNA is then resuspended, electroporated into E. coli DH10B cells, XL1 Blue cells are added and the mixture is plated onto agar plates. After 6 hours at 37° C., the phage plaques are counted and eluted into growth media. Phage stocks are generated when the elutions are clarified by centrifugation and sodium azide is added to 0.2%.

Initial screening of the anti-VH3-23 library is performed by plaque lift essentially as described in Watkins, J. D. et al., (1998) Anal. Biochem., 256:169-177, herein incorporated by reference. Briefly, nitrocellulose filters are coated with goat anti-human kappa antibodies and then blocked with 1% BSA. The filters are then placed on agar plates containing plaques from the phage stock described above and incubated for 18 hours at 22° C. Filters are removed from the plates, rinsed with PBS and incubated with various concentrations of biotinylated germline VH3-23 variable region or PIN variable region known to be VH3-23 type family member. Fab-bound to such variable regions is detected with NeutrAvidin alkaline phosphatase conjugate using a colorimetric substrate. Regions of the agar plate corresponding to the most intense signals are excised, the phages eluted and amplified and reprobed until discreet positive plaques are isolated. Multiple clones are identified and further characterized by ELISA.

Phage stocks of positive clones from the initial screen are used to infect log phase XL1 Blue which are induced with 1 mM IPTG. After 1 hour at 37° C., 15 ml of infected culture is grown for a further 16 hours at 22° C. Cells are pelleted, washed and the periplasmic contents released by the addition of 640 μl of 30 mM Tris pH 8.2, 2 mM EDTA, and 20% sucrose. After 15 minutes at 4° C., the cells are pelleted and the supernatant, containing Fab fragments, is assayed by ELISA. COSTAR #3366 microtiter plates are coated with goat anti-VH3-23 variable region protein at 2 μg/ml in carbonate buffer for 16 hours at 4° C. The wells are blocked with 1% BSA, washed and 0.5 μg/ml VH3-24 variable region protein is added to each well for 1 hour at 22° C. After washing, Fab dilutions are added to the wells for 1 hour at 22° C. Goat anti-human kappa alkaline phosphatase is then added for 1 hour at 22° C. Addition of a colorimetric substrate identified clones with the best binding characteristics.

The best clone is the starting point for the generation of individual CDR libraries. Briefly, each CDR is separately deleted by standard mutagenesis methods. Uridinylated single stranded DNA templates from each CDR-deleted clone are annealed separately with a pool of oligonucleotides which contain all possible amino acids at each position of the CDR, except the amino acids in the CDRs of 3C9. Double stranded DNA is made and libraries generated as described. Screening is done initially by filter lift, positive clones are assayed by ELISA and the DNA sequence determined. The resulting sequences may then be expressed as VH3-23 reactive Mabs or fragments thereof. The above example may be repeated with other clones described in the above examples, such as clone 12C3, which is specific for family member KV4-1; clone 20H5, which is specific for family LV1; clone 15E8, which is specific for family LV2; and clone 4H11, which is cross reactive with VL2 and LV3-25.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, medicine, and molecular biology or related fields are intended to be within the scope of the following claims.

We claim:

1. A panel of family specific antibodies comprising at least four monoclonal antibodies, wherein each of said monoclonal antibodies reacts with at least two members of a variable region family.

2. The panel of claim 1, wherein one of said four monoclonal antibodies is reactive with a light chain variable region framework proteins in the VK3 family, wherein said monoclonal antibody does not cross-react with variable region proteins from non-VK3 families of variable regions.

3. The panel of claim 2, wherein said monoclonal antibody has immunoreactivity with VK3-20 and is unreactive with VK4-1.

4. The panel of claim 1, wherein one of said four monoclonal antibodies is reactive with a heavy chain variable region framework proteins in the VH3 family, wherein said monoclonal antibody does not cross-react with variable region proteins from non-VH3 families of variable regions.

5. The panel of claim 4, wherein said monoclonal antibody has immunoreactivity with VH3-48 and is unreactive with VK4-1.

6. The panel of claim 1, wherein one of said four monoclonal antibodies is reactive with a light chain variable region framework proteins in the VK4 family, wherein said monoclonal antibody does not cross-react with variable region proteins from non-VK4 families of variable regions.

7. The panel of claim 6, wherein said monoclonal antibody has immunoreactivity with VK4-1 and is unreactive with VK3-20, VH3-48, and VH3-23.

8. The panel of claim 1, wherein one of said four monoclonal antibodies is reactive with a light chain variable region framework proteins in the VL1 family, wherein said monoclonal antibody does not cross-react with variable region proteins from non-VL1 families of variable regions.

9. The panel of claim 6, wherein said monoclonal antibody has immunoreactivity with VL1-51 and is unreactive with VK4-1.

10. A panel of antibodies comprising:
a) a first monoclonal antibody having immunoreactivity with VH3-48, said first monoclonal antibody being unreactive with VK3-20, VK4-1, and VH3-23;
b) a second monoclonal antibody having immunoreactivity with VK3-20, said second monoclonal antibody being unreactive with VH3-48, VK4-1, and VH3-23;
c) a third monoclonal antibody having immunoreactivity with VK4-1, said third monoclonal antibody being unreactive with VK3-20, VH3-48, and VH3-23; and
d) a fourth monoclonal antibody having immunoreactivity with VH3-23, said fourth monoclonal antibody being unreactive with VK3-20, VK4-1, and VH3-48.

11. A method for classifying a B-cell non-Hodgkin's lymphoma of a patient, said lymphoma comprising cells expressing an immunologic antigen receptor comprising a variable region, comprising:
a) contacting cells of said lymphoma with the panel of antibodies of claim 11;
b) determining which of said antibodies of said panel bind to said cells;
wherein said lymphoma is classified as belonging to a variable region family corresponding to the variable region recognized by antibodies that bind to said malignancy.

12. A method of classifying a B-cell non-Hodgkin's lymphoma of a patient, said lymphoma comprising cells expressing an immunologic antigen receptor comprising a variable region, the method comprising:
a) obtaining a polynucleotide sequence of said variable region of said lymphoma;
b) comparing said polynucleotide sequence to panel of variable region reference sequences comprising a VH3-48 sequence, a VK3-20 sequence, a VK4-1 sequence, and a VH3-23 sequence
c) identifying the reference sequence having the highest sequence similarity to said variable region of said lymphoma;
wherein said lymphoma is classified as belonging to a variable region family corresponding to the reference sequence having the highest sequence similarity.

13. A method for treating a patient having a B-cell non-Hodgkin's lymphoma of a patient, said lymphoma expressing an immunologic antigen receptor comprising a variable region, comprising:
a) providing the panel of antibodies of claim 11; and
b) treating said patient with a monoclonal antibody selected from said panel.

14. A panel of antibodies comprising:
a) a first monoclonal antibody having immunoreactivity with VH3-48, said first monoclonal antibody being unreactive with VK3-20, VK4-1, and VH3-23;
b) a second monoclonal antibody having immunoreactivity with VK3-20, said second monoclonal antibody being unreactive with VH3-48, VK4-1, and VH3-23;
c) a third monoclonal antibody having immunoreactivity with VK4-1, said third monoclonal antibody being unreactive with VK3-20, VH3-48, and VH3-23;
d) a fourth monoclonal antibody having immunoreactivity with VH3-23, said fourth monoclonal antibody being unreactive with VK3-20, VK4-1, and VH3-48; and
e) a fifth monoclonal antibody having immunoreactivity with VL1-51, said fifth monoclonal antibody being unreactive with VK4-1.


SEQUENCE LISTING (77 sequences)

SEQ ID NO: 1 - protein, 123 aa, Homo sapiens
QLQLQESGPGPVKSSETLSLTCTVSGGTIGSSNYYWGWIRQPPGKGLEWIGSIYYSGGTNYNLSLKSRVTISVDPSKNQFSLQLRSVTAADTAVYYCARICSSLTSYENFDLWGRGTLVTVSS

SEQ ID NO: 2 - protein, 111 aa, Homo sapiens
QSALTQPPSVSGAPGQRVTISCTGSSSNIGTGYDVHWYQQLPGTAPKLLICGNSNRPSGVPDRISGSKSGTSASLAITGLQAEDEADYYCQSYDSSLSGYVFGTGTKVTVL

SEQ ID NO: 3 - protein, 120 aa, Homo sapiens
EVQLLESGGGLVQPGGSLRLSCAASGFTFSGYAMTWVRQAPGKGLKWVSGISGSGGSTYYAASVKGRFTISRDNSKSTLYLQMNSLRAEDTAVYYCAKTINDILTATEDWGQGTLVTVSS

SEQ ID NO: 4 - protein, 114 aa, Homo sapiens
DIVMTQSPDSLAVSLGERATINCKTSQSVLYSSKKKNYLAWYQQKPGQPPKLVIYWASTRGSGVPDRFSGSGSGTDFTLTISSLQAEDVAVYYCQQYYSTPQTFGQGTKVEIKR

SEQ ID NO: 5 - protein, 124 aa, Homo sapiens
QVRLVQSGAEVKKPGASVKISCKTSGYTFINYYIHWVRQAPGQGLEWMGIINPSGGSTTYAQKFQDRVTMTKDMCTNTVYMQLIRLRSEDTALYYCARDVGSGHSHVNYGLDAWGQGTMVNVSS

SEQ ID NO: 6 - protein, 110 aa, Homo sapiens
QSALTQPPSASGSLGQSVTISCTGTSRDIDDSKYVSWYQRHPAKAPQLIIYEVTKRPSGVPDRFSGSKSGNTASLTVSGLQADDEADYYCSSSVHNNSVLFGGGTNLTVL

SEQ ID NO: 7 - protein, 120 aa, Homo sapiens
EVQLVESGGGLVQPGGSLRLSCAGSGFTFSSYEMNWVRQAPGKGLEWISNISSSGDSMYYAGSVKGRFTISRDNAKNSLFLQMNSLRVDDTAIYYCARDSNSWYTSIDYWGQGILVTVSS

SEQ ID NO: 8 - protein, 111 aa, Homo sapiens
QSALTQPASVSGSPGQSIIISCTGTRSDIGTYNRVSWYQHHPGRAPKIITYEVTNRPSGVSYRFSGSKSGNVASLTISGLQAEDEADYYCGSYTTTNTHILFGGGTKLTVL

SEQ ID NO: 9 - protein, 116 aa, Homo sapiens
EGQLVESGGGLVQPGRSLRLSCVDSGFSFSNNWMSWVRQSPGKGLEWVANINEDGSEKHFVDSVKGRFTISRDNVKNSLYLEINSLRVEDTAVYYCVRNNSYFNVWGQGTTVTVSL

SEQ ID NO: 10 - protein, 114 aa, Homo sapiens
DIVMTQSPDSLAVSLGERATINCKSSQTILHRRNNKNYLAWYQQKPGQPPKLLIYWASIRESGVPDRFSGSGSGTDFSLIISSLQPEDVAVYYCQQYNNSLCSFGQGTKLEIKR

SEQ ID NO: 11 - protein, 119 aa, Mus musculus
EVQLQQSGPELEKPGASVKISCKASGYSFTAYNMNWVKQNNGKSLEWIGNIDPYHGGTNYNQKFKGKATLTVDKSSSTAYMQLKSLTSEDYAVYYCARGGGDWSWFAYWGQGTLVTVSA

SEQ ID NO: 12 - DNA, 359 bp, Mus musculus
gaggtccagctgcagcagtctggacctgagctggagaagcctggcgcttcagtgaagatatcctgcaaggcttctggttattcattcacagcctacaacatgaactgggtgaagcagaacaatggaaagagccttgagtggattggaaatattgatccttaccatggtgggactaactacaaccagaagttcaagggcaaggccacattgactgtagacaaatcctccagcacagcctatatgcagctcaagagcctgacatctgaggactatgcagtctattattgtgcaagaggggggggggactggtcctggtttgcttattggggccaagggacactggtcactgtctctgcagc

SEQ ID NO: 13 - protein, 108 aa, Mus musculus
DIVMTQSHKFMSTSVGDRVSITCKASQDVNTAVAWYQQKPGQSPKVLIYWASNRHTGVPDRFTGSGSGTDYTLTISSMQAEDLALYYCHQHYSTPWTFGGGTKLEIER

SEQ ID NO: 14 - DNA, 324 bp, Mus musculus
gacattgtgatgacccagtctcacaaattcatgtccacatcagtaggagacagggtcagcatcacctgcaaggccagtcaggatgtgaatactgctgtagcctggtatcaacagaaaccaggacaatctcctaaagtactgatttactgggcatccaaccggcacactggagtccctgatcgcttcacaggcagtggatctgggacagattatactctcaccatcagcagtatgcaggctgaggacctggcactttattactgtcatcaacattatagcactccgtggacgttcggtggaggcaccaagctggaaatcgaacgg

SEQ ID NO: 15 - protein, 115 aa, Mus musculus
EVQLVESGGGLVKPGGSLKLSCTASGFTFSSYAMSWVRQSPEKRLEWVAEISSGGIHTYYSDTVTGRFSISRDNAKNSTLYLEMNSLRSEDTAIYYCARETTGTWGQGTLVTVSA

SEQ ID NO: 16 - DNA, 342 bp, Mus musculus
gaagtgcagctggtggagtctgggggaggcttagtgaagcctggagggtccctgaaactctcctgtacagcctctggattcactttcagtagctatgccatgtcttgggttcgccagtctccagagaagaggctggagtgggtcgcagaaattagtagtggaggtattcacacctactattcagacactgtgacgggccgattctccatctccagagacaatgccaagaacaccctgtacctggaaatgaacagtcttaggtctgaggacacggccatttattactgtgcaagggagacaactgggacgtggggccaagggactctggtcactgtctctgca

SEQ ID NO: 17 - protein, 107 aa, Mus musculus
QIVLTQSPAIMSASPGEKVTMTCSVSSSITYMHWFRQKPGTSPKLWIYTTSNLASGVPARFSGSGSGTSYSLTISRMEAEDAATYYCQQRSRYPFTFGSGTKLEIKR

SEQ ID NO: 18 - DNA, 321 bp, Mus musculus
caaattgttctcacccagtctccagcaatcatgtctgcatctccaggggagaaggtcaccatgacctgcagtgtcagctcaagtataacttacatgcactggttccggcagaagccaggcacttctcccaaactctggatttataccacatccaacctggcttctggagtccctgctcgcttcagtggcagtggatctgggacctcttattctctcacaatcagccgaatggaggctgaagatgctgccacttattactgccagcaaaggagtcgttacccatttacgttcggctcggggacaaagttggagataaaacgg

SEQ ID NO: 19 - protein, 120 aa, Mus musculus
EVKLVESGGGLVQPGGSLSLSCAASGFTYTDYYMNWVRQPPGKALEWLALIRNKANGYTTEYSASLKGRFTISRDNSQSILYLQMNALRAEDSATYYCVRENINYYFDYWGQGTTLTVSS

SEQ ID NO: 20 - DNA, 360 bp, Mus musculus
gaggtgaagctggtggaatctggaggaggtttggtacagccggggggttctctgagtctctcctgtgcagcttctggattcacctacactgattactacatgaactgggtccggcagcctccagggaaggcactagagtggttggctttgattagaaacaaagctaatggctacacaacagagtacagtgcatctttgaagggtcggttcaccatctccagagataattctcaaagcatcctctatcttcaaatgaatgccctgagagctgaggacagtgccacttattactgtgtaagagaaaatattaactactactttgactactggggccaaggcaccactctcacagtctcctca

SEQ ID NO: 21 - protein, 112 aa, Mus musculus
DIVLTQSPASLAVSLGQRATISCRASESVEYFGASLMQWYQQKSGQPPKLLIRAATNVESGVPARFSGSGSGTDFSLNIHPVEEDDIAMYFCQQSRKVPWTFGGGTKLEIKR

SEQ ID NO: 22 - DNA, 338 bp, Mus musculus
gacattgtactcacccaatctccagcttctttggctgtgtctctagggcagagagccaccatctcctgtagagcctctgaaagtgttgaatattttggcgcaagtttaatgcagtggtaccaacagaaatcaggacagccacccaaactcctcatccgtgctgcaaccaacgttgaatctggggtccccgccaggtttagtggcagtgggtctgggacagacttcagcctcaacatccatcctgtggaggaggatgatattgcaatgtatttctgtcagcaaagtaggaaggttccttggacgttcggtggaggcaccaagctggaaatcaaacgggc

SEQ ID NO: 23 - protein, 120 aa, Mus musculus
QVQLKESGPDLVAPSQSLSITCTVSGFSLTSYGVHWVRQPPGKGLEWLVVIWSDGSTTNNSPLKSRLSISKDNSKSQVFLKMNSLQTDDTAMYYCARHWGTTVVDAMDYWGQGTSVTVSS

SEQ ID NO: 24 - DNA, 360 bp, Mus musculus
caggtgcagctgaaggagtcaggacctgacctggtggcgccctcacagagcctgtccatcacatgcaccgtctcagggttctcattaaccagctatggtgtacactgggttcgccagcctccaggaaagggtctggagtggctggtagtgatatggagtgacggaagcacaaccaataattcacctctcaaatccagactgagcatcagcaaggacaactccaagagccaagttttcttaaaaatgaacagtctccaaactgatgacacagccatgtactactgtgccagacattggggcacgacggtagtggatgctatggactactggggtcaaggaacctcagtcaccgtctcctca

SEQ ID NO: 25 - protein, 109 aa, Mus musculus
DIQMTQTTSSLSASLGDRVIISCRASQDIRNYLNWYQQKPDGTVKLLIYYTSQLHSGVPSRFSGSGSGTDYSLTINNLSEQEDIATYFCQQYDTLPWTFGGGTKLEIKR

SEQ ID NO: 26 - DNA, 328 bp, Mus musculus
gatatccagatgacacagactacatcctccctgtctgcctctctgggagacagagtcatcatcagttgcagggcaagtcaggacattagaaattatttaaactggtatcagcagaaaccagatggaactgttaaactcctgatctactacacatcacaattacactcaggagtscccatcaaggttcagtggcagtgggtctggaacagattattctctcasccattaacaacctggagcaagaagatattgccacttacttttgccaacagtatgatacgcttccgtggactttcggtggaggcaccaagctggagatcaaacgggc

SEQ ID NO: 27 - protein, 120 aa, Mus musculus
QVQLQQPGAELVMPGASVKMSCKASGYTFTDYWMHWVKQRPGQGLEWIGTIDTSDSYTNYNQKFKGKATLTVDESSSTAYMQLSSLTSEDSAVYYCVRWKKYGNYFFDYWGQGTTLTVSS

SEQ ID NO: 28 - DNA, 360 bp, Mus musculus
caggtccaactgcagcagcctggggctgagcttgtgatgcctggggcttcagtgaagatgtcctgcaaggcttctggctacacattcactgactactggatgcactgggtgaagcagaggcctggacaaggccttgagtggatcggaacgattgatacttctgatagttatactaattacaatcaaaagttcaagggcaaggccacattaactgtagacgaatcctccagcacagcctacatgcagctcagcagcctgacatctgaggactctgcggtctattattgtgtaagatggaaaaagtatggtaactacttttttgactactggggccaaggcaccactctcacagtctcctca

SEQ ID NO: 29 - protein, 108 aa, Mus musculus
DIVMTQSHKFMSTSVGDRVSITCKASQDVNTAVAWYQQKPGQSPKLLIYWASTRHTGVPDRFTGSGSGTDFSLTISSVQAEDLGLYYCQQCYSTPYAFGGGTKLEIKR

SEQ ID NO: 30 - DNA, 326 bp, Mus musculus
gacattgtgatgacccagtctcacaaattcatgtccacatcagtaggagacagggtcagcatcacctgcaaggccagtcaggatgtgaatactgctgtagcctggtatcaacaaaaaccagggcaatctcctaaactactgatttactgggcatccacccggcacactggagtccctgatcgcttcacaggcagtggatctgggacagattttagtctcaccatcagcagtgtgcaggctgaagacctgggactttattactgtcagcaatgttatagcactccgtacgcgttcggaggggggaccaagctggaaataaaacgggc

SEQ ID NO: 31 - protein, 118 aa, Mus musculus
EVKLVESGGGLVKPGGSLKLSCAASGFAFSNYDMSWVRQTPEKRLEWVATIRSDDSYTYYPDSVKGRFTISRDNARNTLYLQMSSLRSEDTAFYYCARREGNYAMDYWGQGTSVTVSS

SEQ ID NO: 32 - DNA, 354 bp, Mus musculus
gaagtgaagctggtggagtctgggggaggcttagtgaagcctggagggtccctgaaactctcctgtgcagcctctggattcgctttcagtaactatgacatgtcttgggttcgccagactccggagaagaggctggagtgggtcgcaaccattagaagtgatgatagttacacctactatccagacagtgtgaagggccgattcaccatctccagagacaatgccaggaacaccctgtacctgcaaatgagcagtctgaggtctgaggacacggccttctattactgtgcaagacgggagggtaactatgctatggattactggggtcaaggaacctcagtcaccgtctcctca

SEQ ID NO: 33 - protein, 108 aa, Mus musculus
DIQMTQSPASLSVSVGETVTITCRASENIYSYLAWYQQKKGKSPQLLVYAATNLGDGVPTRFSGSGSGTQYSLKINSLQSEDFGSYYCQHFWGNPLTFGAGTKLELKR

SEQ ID NO: 34 - DNA, 324 bp, Mus musculus
gacatccagatgactcagtctccagcctccctatctgtatctgtgggagaaactgtcaccatcacatgtcgagcaagtgagaatatttacagttatttagcatggtatcagcagaaaaagggaaaatctcctcagctcctggtctatgctgcaacaaacttaggagatggtgtgccaacaaggttcagtggcagtggatcaggcacacagtattccctcaagatcaacagcctgcagtctgaagattttgggagttattactgtcaacatttttggggtaacccgctcacgttcggtgctgggaccaagctggagctgaaacgg

SEQ ID NO: 35 - DNA, 21 bp, Artificial Sequence
  Synthetic
tcctgtgcgaggcagccaacg

SEQ ID NO: 36 - DNA, 24 bp, Artificial Sequence
  Synthetic
gcctgagttccacgacaccgtcac

SEQ ID NO: 37 - DNA, 21 bp, Artificial Sequence
  Synthetic
tgtccgctttcgctccaggtc

SEQ ID NO: 38 - DNA, 33 bp, Artificial Sequence
  Synthetic
ccactgtattttggcctctctgggatagaagtt

SEQ ID NO: 39 - DNA, 20 bp, Artificial Sequence
  Synthetic
gctcccgggtagaagtcact

SEQ ID NO: 40 - DNA, 26 bp, Artificial Sequence
  Synthetic
tctagaattcacgcgtcccccccccc

SEQ ID NO: 41 - DNA, 28 bp, Artificial Sequence
  Synthetic
tctagaattcacgcgtcccccccccccc

SEQ ID NO: 42 - DNA, 24 bp, Artificial Sequence
  Synthetic
caacggccacgctgctcgtatccg

SEQ ID NO: 43 - DNA, 24 bp, Artificial Sequence
  Synthetic
gtagtccttgaccaggcagcccag

SEQ ID NO: 44 - DNA, 21 bp, Artificial Sequence
  Synthetic
ggctcctgggggaagaagccc

SEQ ID NO: 45 - DNA, 30 bp, Artificial Sequence
  Synthetic
gaagttattcagcaggcacacaacagaggc

SEQ ID NO: 46 - DNA, 26 bp, Artificial Sequence
  Synthetic
cacaccagtgtggccttgttggcttg

SEQ ID NO: 47 - DNA, 24 bp, Artificial Sequence
  Synthetic
ggggaaaagggttggggcggatgc

SEQ ID NO: 48 - DNA, 21 bp, Artificial Sequence
  Synthetic
aggctcagcgggaagaccttg

SEQ ID NO: 49 - DNA, 34 bp, Artificial Sequence
  Synthetic
ggttccggacttaagctgctcatcagatggcggg

SEQ ID NO: 50 - DNA, 29 bp, Artificial Sequence
  Synthetic
ggcgccgccttgggctgacctaggacggt

SEQ ID NO: 51 - DNA, 25 bp, Artificial Sequence
  Synthetic
agggaaataacctttgaccaggcat

SEQ ID NO: 52 - DNA, 23 bp, Artificial Sequence
  Synthetic
ctagacagggatccagagttcca

SEQ ID NO: 53 - DNA, 24 bp, Artificial Sequence
  Synthetic
acgactgaggcacctccagatgtt

SEQ ID NO: 54 - DNA, 22 bp, Artificial Sequence
  Synthetic
tggggtagaagttgttcaagaa

SEQ ID NO: 55 - DNA, 26 bp, Artificial Sequence
  Synthetic
tctagaattcacgcgtcccccccccc

SEQ ID NO: 56 - DNA, 28 bp, Artificial Sequence
  Synthetic
tctagaattcacgcgtcccccccccccc

SEQ ID NO: 57 - DNA, 19 bp, Artificial Sequence
  Synthetic
caggggccagtggatagac

SEQ ID NO: 58 - DNA, 24 bp, Artificial Sequence
  Synthetic
gatggtgggaagatggatacagtt

SEQ ID NO: 59 - DNA, 16 bp, Artificial Sequence
  Synthetic
gtaaaacgacggccag

SEQ ID NO: 60 - DNA, 17 bp, Artificial Sequence
  Synthetic
caggaaacagctatgac

SEQ ID NO: 61 - DNA, 37 bp, Artificial Sequence
  Synthetic
tctagaattcacgcgtccaccatgaactttgggctga

SEQ ID NO: 62 - DNA, 38 bp, Artificial Sequence
  Synthetic
gaggggcccttggtcgacgctgaggagacggtgactga

SEQ ID NO: 63 - DNA, 38 bp, Artificial Sequence
  Synthetic
tctagaattcacgcgtccaccatgagtgtgcccactca

SEQ ID NO: 64 - DNA, 36 bp, Artificial Sequence
  Synthetic
tgcagccacagtccgtttcagctccagcttggtccc

SEQ ID NO: 65 - DNA, 36 bp, Artificial Sequence
  Synthetic
gagctgaaacggactgtggctgcaccttctgtcttc

SEQ ID NO: 66 - DNA, 21 bp, Artificial Sequence
  Synthetic
aggcctgtacggaagtgttac

SEQ ID NO: 67 - protein, 122 aa, Artificial Sequence
  Synthetic
QVQLQQWGAGLLKPSETLSLICAVYGGSFSGYSWSWIRQPPGKGLEWIGEINHRGSTNYNPSLKSRVTISVDTSKNQFSLKVRSVTAADTAVYYCARGDLGNNGRPARPDYWGQGTLVTVSS

SEQ ID NO: 68 - protein, 109 aa, Artificial Sequence
  Synthetic
EVVLTQSPATLSLSPGERATLSCRASQSVSSYLAWYQQKPGQAPRLLIYDAFNRATGIPARFSGSGSGTDFTLTISSLEPEDFAVYFCQQRGNWPPLTFGGGTKVEIKR

SEQ ID NO: 69 - protein, 116 aa, Artificial Sequence
  Synthetic
DMELVESGGGLVQPGGSLRLSCVGSGFTFNSFWMTWVRQAPGKGLEWVANITRDGSEKYYRDSVKGRFTISRDNGKKSLYLQMNSLKTEDTAIYYCAREYYGMDLWGQGTTVTVSS

SEQ ID NO: 70 - protein, 109 aa, Artificial Sequence
  Synthetic
DIQMTQSPSTLSASVGDRVTITCRASQSISDWLAWYQQKPGKAPKLLIYKASSLESGVPSRFSGSGSGTEFTLTISSLQPDDFATYYCQQYKSYEGFTFGPGTKVDIRR

SEQ ID NO: 71 - protein, 118 aa, Artificial Sequence
  Synthetic
EVQLVESGGGLVQPGGSLRLSCAASGFTFSGYSMNWVRQAPGKGLEWVSYISSSSTTIYYADSVKGRITISRDNAKNSLYLQMNTLRADDTAVYYCARVKGVGMADYWGQGTLVTVSS

SEQ ID NO: 72 - protein, 110 aa, Artificial Sequence
  Synthetic
EIVLTQSPGTLSLSPGERATLSCRASQSISSSYLAWYQQKPGQAPRLLIYGASSRATGIPDRFSGGGSGTDFTLTIIRLEPEDFAVYYCQQYGGSPRNTFGQGTKLEIKR

SEQ ID NO: 73 - protein, 126 aa, Artificial Sequence
  Synthetic
QVQLQESGPGLLKPSETLSLTCTVSGGSISSYSWSWIRQPPGKVLEWIGNISDSGSTNYNPSLKSRVTISVDTSKNQFSLKVNSVTAADTAVYYCARDSTTTTPGSHYRDLTLDVWGQGTTVTVSS

SEQ ID NO: 74 - protein, 108 aa, Artificial Sequence
  Synthetic
DIQMTQSPSSLSASVGDRVTITCRASQSIGSYLNWYQQKPGKAPELLIYDASSLQSGAPSRFSGSGSGTDFTLTISSLQPEDFATYYCHQSYSAPRTFGQGTKLEIKR

SEQ ID NO: 75 - protein, 122 aa, Homo sapiens; identical to SEQ ID NO: 67

SEQ ID NO: 76 - protein, 109 aa, Homo sapiens; identical to SEQ ID NO: 68

SEQ ID NO: 77 - DNA, 24 bp, Artificial Sequence
  Synthetic
tctagaattcacgcgtcccccccc